(19) United States
(12) Patent Application Publication
(10) Pub. No.: US 2026/0358330 A1
(43) Pub. Date: Nov. 12, 2026

(54) CELL CULTURE WITHIN MICROFLUIDIC STRUCTURES

(71) Applicant: Cellanome, Inc., Foster City, CA (US)

(72) Inventors: Yir-Shyuan WU, Pao Alto, CA (US); Tarun Kumar Khurana, Palo Alto, CA (US); Yunmin Li, Milbrae, CA (US); Seyedsina Moeinzadeh, Mountain View, CA (US)

(21) Appl. No.: 19/630,294

(22) Filed: Mar. 26, 2026

Related U.S. Application Data
(63) Continuation of application No. PCT/US2024/048730, filed on Sep. 26, 2024.
(60) Provisional application No. 63/585,930, filed on Sep. 27, 2023.

(51) Int. Cl. G01N 33/50

(52) U.S. Cl. CPC G01N 33/505

(57) ABSTRACT

The systems and methods described herein are directed to carrying out one or more highly multiplexed cellular assays. In some embodiments, one or more channels of a fluidic device are provided with photopolymerizable polymer precursors and cells randomly disposed on a surface, after which positions of cells are measured by a detector, hydrogel chambers are synthesized by photopolymerization to enclose individual cells, and channels are loaded with assay reagents. Assay signals indicative of desired cellular characteristics are generated for each of the enclosed cells.

### CROSS-REFERENCE

This application is a continuation of International Patent Application No. PCT/US2024/048730, filed Sep. 26, 2024, which claims the benefit of U.S. Provisional Application No. 63/585,930 filed Sep. 27, 2023, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Engineered cell-based therapeutics provide promising new approaches to treating complex diseases because of a cell's ability to sense and integrate a wide range of signals, to actively move to specific tissue compartments, and to actuate context-dependent responses, e.g. Fischbach et al, Science Transl. Med., 5:1797 (2013). Such cell-based approaches provide novel therapeutic devices that address current obstacles faced by small molecules and biologics, such as poor target specificity, undesirable tissue compartment localization, a lack of personalization, and limited potential for the effects of the drug to be modified once administered to a patient, either over space and time, or in response to a changing clinical picture. These issues can reduce the pharmaceutical utility of such compounds. Cytotoxic lymphocytes (CLs), such as cytotoxic T-lymphocytes (CTLs) and Natural Killer cells (NKs), are an excellent platform for engineering cell-based therapeutic systems for several reasons: (i) cytotoxic lymphocytes possess a unique delivery-cell-to-target-cell molecular transfer system in the granzyme-perforin pathway; (ii) T-cell receptors (TCRs), or the related chimeric antigen receptors (CARs), endow cytotoxic lymphocytes with an exquisite level of specificity in targeting a cell population presenting Major Histocompatibility Complex (MHC) bound cognate antigen or, in the case of CARs, an arbitrary surface antigen; (iii) activated cytotoxic lymphocytes differentially express cytokine and tissue specific receptors, enabling selective lymphocyte homing throughout the body to target tissue; and (iv) laboratory and clinical protocols for lymphocyte modification and therapeutic administration have been developed in the field of adoptive cell therapy, e.g., Restifo et al, Nature Reviews Immunology, 12:269-281 (2012). In view of these advantages and positive clinical results, a host of cell-based therapies have been approved to treat a range of cancers and other disorders.

However, along with the successes of cell-based therapies there are significant development, manufacturing and quality assurance challenges due to the complexities of using living organisms as drugs. Tests must be carried out for selecting an appropriate cellular subpopulation to engineer and for assuring at each step of the manufacturing process that there is no deleterious change in the identity, purity, or operability of the engineered subpopulation, e.g. Tanna et al, Cytotherapy, 21:278-288 (2019); Wang et al, Molecular Therapy, 3: 16015 (2016); Levine et al, Molecular Therapy: Methods & Clinical Development, 4:92-101 (2017). Accordingly, the field of cell-based therapies would be advanced by the availability of a cellular analysis platform, including methods and systems, for performing a wide range of highly multiplexed cellular assays relevant to the development and manufacture of cell-based therapeutics.

### SUMMARY

It is often valuable to perform cell assays on individual cells to characterize the heterogeneity of the cell population and identify characteristics of potent cells in the population. These assays can be performed in microstructured devices that compartmentalize individual cells. During or after the process of assaying these cells, there is a need to culture these individual cells with desirable characteristics into larger clonal populations.

The systems and methods described herein are directed to methods and systems for performing large-scale multiplexed single cell assays, particularly for characterizing populations engineered cells developed for medical or industrial applications, such as cell-based therapeutics. Example single cell assays include, but are not limited to, cytotoxicity, proliferative capacity, activation status, vector copy number and insertion site analysis of transformed cells, and the like.

In some embodiments, methods described herein comprise: synthesizing one or more hydrogel chambers enclosing each of one or more cells disposed on a surface of a channel; and performing one or more assays on the cells in the channels to determine one or more of the cellular characteristics. In some embodiments, synthesizing one or more hydrogel chambers comprises (a) providing a fluidic device comprising (i) one or more channels each comprising a first surface, (ii) a spatial energy modulating element in optical communication with each first surface, and (iii) a detector that identifies positions of cells in each channel based on one or more optical signals therefrom; (b) loading each channel with cells and one or more polymer precursors so that the cells are disposed on or adjacent to at least one of the first surfaces, wherein the cells may be loaded separately or together with the one or more polymer precursors; and (c) synthesizing one or more chambers in at least one channel, wherein each chamber encloses at least one cell, by projecting light into the channel with the spatial energy modulating element such that the projected light causes cross-linking of the one or more polymer precursors to form polymer matrix walls of the chambers, wherein the positions of the synthesized chambers are determined in each channel by the positions of the cells enclosed thereby identified by the detector. In some embodiments, performing one or more assays comprises loading one or more channels with assay reagents; and incubating the cells in each channel under assay conditions to generate signals from chambers of each channel indicative of a cellular characteristic. In some embodiments, such cellular characteristics are selected from, but are not limited to, cytotoxicity, viability, proliferation rate, phenotype, vector copy number, vector integration sites, transcriptome, and genomic copy number variation.

In some embodiments, methods described herein comprise (a) synthesizing one or more hydrogel chambers enclosing each of one or more cells disposed on a surface of a channel; (b) lysing the cells so that genomic DNA of each cell is released into its hydrogel chamber; (c) amplifying the genomic DNA of each cell; and (d) performing an assay to determine a characteristic of the amplified genomic DNA. In some embodiments, such determining comprises loading one or more channels with assay reagents; and incubating the genomic DNA in each channel under assay conditions to generate signals from chambers of each channel indicative of a characteristic of the genomic DNA. In some embodiments, such characteristics comprise integrated viral copy number, location of viral integration sites, or genomic copy number variation.

In some embodiments, assays carried out in each channel may be the same or different than assays carried out in other channels of the same fluidic device. In some embodiments, polymer matrix walls of chambers have a predetermined semi-porosity that prevents passage of cells and permits passage of molecules below a predetermined size. In some embodiments, polymer matrix walls of chambers may be degradable, or depolymerizable, either generally throughout a channel or on-demand on a chamber-by-chamber basis.

In some embodiments, the system is directed to measuring single cell characteristics of a population of cells comprising the elements of: (a) one or more channels each comprising a first surface, a plurality of cells disposed on the first surface, and one or more polymer precursors; (b) at least one spatial energy modulating element in optical communication with the first surface of each channel; (c) at least one detector in optical communication with the first surface of each channel and in operable association with at least one spatial energy modulating element, a detector detecting each of the plurality of cells and determining a position thereof on the first surface of at least one channel; and (d) a plurality of gel chambers in each channel, wherein each gel chamber encloses a single cell of the plurality of cells, wherein the gel chambers are synthesized by projecting light into the channel with a spatial energy modulating element such that the projected light causes cross-linking of the one or more polymer precursors to form polymer matrix walls of the chambers, and wherein the positions of the synthesized chambers are determined by the positions of cells enclosed thereby identified by the detector. In some embodiments, cells are randomly disposed on the first surface of at least one channel.

In some of the above-described embodiments, chamber walls comprise a degradable hydrogel. In some embodiments, methods comprise degrading chamber walls prior to loading assay reagents.

In an aspect, provided herein is a method of analyzing cells, the method comprising: loading a channel with: (i) one or more cells of a first type, (ii) one or more cells of a second type, and (iii) one or more polymer precursors; polymerizing the one or more polymer precursors to form a chamber that encloses the one or more cells of the first type and the one or more cells of the second type, wherein the chamber is within the channel, wherein the one or more cells of the first type and the one or more cells of the second type both cannot flow through a wall of the chamber from an interior of the chamber to an exterior of the chamber; after the polymerizing of the one or more polymer precursors to form the chamber, loading the channel with a medium containing a component; and after loading the channel with the medium, measuring a produced antigen from either of the one or more cells of the first type and/or one or more cells of the second type in the chamber.

In some cases, the one or more cells of the first type comprise antigen receiving cells. In some cases, the one or more cells of the first type comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs). In some cases, the one or more cells of the first type comprise T-cells. In some cases, the one or more cells of the first type comprise OT1 cells.

In some cases, the one or more cells of the second type comprise antigen receiving cells. In some cases, the one or more cells of the second type comprise activated OT1 cells, activated T cells, activated B cells, macrophages, dendritic cells, monocytes, astrocytes, microglia, stromal cells, feeder cells, cancer associated fibroblast (CAF), or tumor associated macrophages (TAM). In some cases, the one or more cells of the second type comprise dendritic cells. In some cases, the one or more cells of the first type are the same type of cells as the one or more cells of the second type.

In some cases, the component is selected from the group consisting of an activator, inhibitor, activated cell, non-activated cell, growth factor, and combinations thereof. In some cases, the component comprises activated OT1 cells, and the activated OT1 cells cannot pass through the wall of the chamber from the exterior of the chamber to the interior of the chamber.

In some cases, the activated OT1 cells secrete a signaling chemical that interacts with the one or more cells of the first type or the one or more cells of the second type within the chamber to have an increased cell life compared to a situation where there is an absence of the activated OT1 cells proximate to the exterior of the chamber. In some cases, the signaling chemical comprises a growth factor.

In some cases, the method further comprises measuring one or more characteristics of the cell. In some cases, the one or more characteristics are selected from the group consisting of (i) a change in total number of cells of the first type and/or the second type during a time duration, (ii) an activation of the one or more cells of the first type and/or the second type during a time duration, (iii) a change in the total number of live cells of the first type and/or the second type during a time duration, (iv) a change in the total number of dead cells of the first type and/or the second type during a time duration, and (v) combinations thereof.

In some cases, the one or more cells of the first type physically contact with the one or more cells of the second type within the chamber to form an activated state of either the one or more cells of the first type and/or the one or more cells of the second type.

In some cases, the dendritic cells comprise express green fluorescent protein (GFP) and the OT1 cells are in an activated state, wherein the OT1 cell in the chamber produce the antigen. In some cases, the antigen is a CD71 antigen.

In some cases, the loading the channel with the medium comprises flowing non-activated OT1 cells and phorbol myristate acetate (PMA).

In some cases, the method further comprises: after the polymerizing of the one or more polymer precursors, measuring a first total number of cells of the first type or second type within the chamber; and after a predetermined time period, measuring a second total number of cells of the first type or second type within the chamber.

In some cases, the produced antigen is CD69, CD71, CD25, CD107a, 4-1BB, PD-1, CD80, CD86 and CD83, CD34, CD133, SSEA4, TRA-1-60, EpCAM, or CA9. In some cases, the produced antigen is CD71. In some cases, the component secretes a signaling chemical that permeates through a wall of the chamber into the interior of the chamber.

In another aspect, provided herein is a method of culturing cells, the method comprising: loading a channel with one or more cells and one or more polymer precursors; polymerizing the one or more polymer precursors to form a chamber that encloses the one or more cells, wherein the chamber is within the channel, wherein the one or more cells cannot flow through a wall of the chamber from an interior of the chamber to an exterior of the chamber; after the polymerizing of the one or more polymer precursors to form the chamber, loading the channel with a medium containing a component; and after loading the channel with the medium, measuring a characteristic of the one or more cells enclosed in the chamber.

In some cases, the characteristic corresponds to a total number of the cells increasing within the chamber through cell division after the loading of the channel with the medium. In some cases, the one or more cells comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs), or any combination thereof. In some cases, the one or more cells comprise T-cells. In some cases, the one or more cells comprise OT1 cells.

In some cases, the component is selected from the group consisting of an activator, inhibitor, activated cell, non-activated cell, growth factor, and combinations thereof. In some cases, the component comprises activated OT1 cells, activated T cells, activated B cells, macrophages, dendritic cells, monocytes, astrocytes, microglia, stromal cells, feeder cells, cancer associated fibroblast (CAF), or tumor associated macrophages (TAM). In some cases, the component comprises activated OT1 cells, and the activated OT1 cells cannot pass through the wall of the chamber from the exterior of the chamber to the interior of the chamber.

In some cases, the activated OT1 cells secrete a signaling chemical that interacts with the one or more cells within the chamber to have an increased cell life compared to a situation where there is an absence of the activated OT1 cells proximate to the exterior of the chamber. In some cases, the signaling chemical comprises a growth factor.

In some cases, the characteristic is selected from the group consisting of (i) a change in total number of cells during a time duration, (ii) an activation of the one or more cells during a time duration, (iii) a change in the total number of live cells during a time duration, (iv) a change in the total number of dead cells during a time duration, and (v) combinations thereof. In some cases, the characteristic is expression of CD69, CD71, CD25, CD107a, 4-1BB, PD-1, CD80, CD86 and CD83, CD34, CD133, SSEA4, TRA-1-60, EpCAM, or CA9. In some cases, the one or more OT1 cells in the chamber produce a CD71 antigen.

In some cases, the loading the channel with the medium comprises flowing non-activated OT1 cells and phorbol myristate acetate (PMA). In some cases, the method further comprises: after the polymerizing of the one or more polymer precursors, measuring a first total number of cells within the chamber; and after a predetermined time period, measuring a second total number of cells within the chamber.

In an aspect, provided herein is a method of analyzing cells, the method comprising: loading a channel with (i) one or more cells of a first type, (ii) one or more cells of a second type, and (iii) one or more polymer precursors; polymerizing the one or more polymer precursors to form a first chamber that encloses the one or more cells of the first type, wherein the first chamber is within the channel; polymerizing the one or more polymer precursors to form a second chamber that encloses the one or more cells of the second type, wherein the second chamber is within the channel, wherein the one or more cells of the first type cannot flow through a wall of the first chamber from an interior of the first chamber to an exterior of the first chamber, wherein the one or more cells of the second type cannot flow through a wall of the second chamber from an interior of the second chamber to an exterior of the second chamber; after the polymerizing of the one or more polymer precursors to form the first chamber and the second chamber, loading the channel with a medium containing a component; and after loading the channel with the medium, measuring a characteristic of the one or more cells of the first type in the first chamber and/or the one or more cells of a second type in the second chamber.

In some cases, the first chamber and the second chamber are disposed in a spaced apart arrangement having a separation distance ranging from 5 to 1000 microns. In some cases, the first chamber and the second chamber each have an inner diameter ranging from 10 to 500 microns. In some cases, the second chamber has an inner diameter that is 2 to 10 times greater than an inner diameter of the first chamber.

In another aspect, provided herein is a method of analyzing cells, the method comprising: flowing one or more cells of a first type into a channel, wherein the channel comprises a fluidic structure configured to trap the flowing one or more cells of the first type at the fluidic structure; flowing one or more cells of a second type into the channel along with a medium, wherein the one or more cells of the second type are not trapped by the fluidic structure; and measuring a characteristic of the one or more cells of a first type and/or one or more cells of a second type.

In some cases, the fluidic structure is configured to at least partially enclose the one or more cells of the first type. In some cases, the fluidic structure comprises a C-shaped structure to trap the one or more cells of the first type. In some cases, the fluidic structure comprises an array of pillars configured to trap the one or more cells of the first type. In some cases, the channel comprises a first surface and second surface. In some cases, in an axis perpendicular to the first surface and/or the second surface, the fluidic structure does not touch both the first surface and second surface. In some cases, the first surface and the second surface are disposed opposite each other across a channel of the fluidic device. In some cases, the fluidic structure comprises a non-porous polymer material. In some cases, the fluidic structure comprises a porous hydrogel polymer material configured to partially enclose the one or more cells of the first type.

In some cases, the one or more cells of the first type comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs), or any combination thereof. In some cases, the one or more cells of the second type comprise activated OT1 cells, activated T cells, activated B cells, macrophages, dendritic cells, monocytes, astrocytes, microglia, stromal cells, feeder cells, cancer associated fibroblast (CAF), or tumor associated macrophages (TAM), or any combination thereof.

In another aspect, provided herein is a method of analyzing one or more cells, the method comprising: loading a fluidic device with: (i) the one or more cells and (ii) one or more polymer precursors; polymerizing the one or more polymer precursors to form a chamber within the fluidic device that encloses a cell of the one or more cells, wherein the cell of the one or more cells cannot flow through a wall of the chamber from an interior of the chamber to an exterior of the chamber; loading the fluidic device with a first medium containing a component; and after loading the fluidic device with the medium, measuring a produced antigen from the cell in the chamber.

In some cases, the one or more cells comprise antigen receiving cells. In some cases, the one or more cells comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs), or any combination thereof. In some cases, the one or more cells comprise T-cells. In some cases, the one or more cells comprise OT1 cells.

In some cases, the method further comprises introducing one or more additional cells into the fluidic device. In some cases, after the polymerizing of the one or more polymer precursors, an additional cell of the one or more additional cell is enclosed in the chamber. In some cases, the one or more additional cells comprise antigen receiving cells. In some cases, the one or more additional cells comprise activated OT1 cells, activated T cells, activated B cells, macrophages, dendritic cells, monocytes, astrocytes, microglia, stromal cells, feeder cells, cancer associated fibroblast (CAF), or tumor associated macrophages (TAM). In some cases, the one or more additional cells comprise dendritic cells. In some cases, the one or more cells are the same type of cells as the one or more additional cells.

In some cases, the component is selected from the group consisting of an activator, inhibitor, activated cell, non-activated cell, growth factor, and combinations thereof. In some cases, the component comprises activated OT1 cells, and the activated OT1 cells cannot pass through the wall of the chamber from the exterior of the chamber to the interior of the chamber. In some cases, the activated OT1 cells secrete a signaling chemical that interacts with the cell in the chamber such that the cell has an increased cell life compared to a situation where there is an absence of the activated OT1 cells proximate to the exterior of the chamber. In some cases, the signaling chemical comprises a growth factor.

In some cases, the method further comprises measuring one or more characteristics of the cell. In some cases, the one or more characteristics are selected from the group consisting of (i) a change in total number of cells during a time duration, (ii) an activation of the one or more cells or the one or more additional cells during a time duration, (iii) a change in the total number of live cells of the one or more cells or the one or more additional cells during a time duration, (iv) a change in the total number of dead cells of the one or more cells or the one or more additional cells during a time duration, and (v) combinations thereof.

In some cases, the cell physically contacts the additional cell within the chamber to form an activated state of either the cell or the additional cell. In some cases, the dendritic cells express green fluorescent protein (GFP), and the GFP stimulates the cell to produce the antigen. In some cases, the cell comprises an OT1 cell. In some cases, the antigen comprises a CD71 antigen.

In some cases, the medium comprises non-activated OT1 cells and phorbol myristate acetate (PMA). In some cases, the method further comprises: after the polymerizing of the one or more polymer precursors, measuring a first total number of cells within the chamber; and after a predetermined time period, measuring a second total number of cells within the chamber.

In some cases, the produced antigen is CD69, CD71, CD25, CD107a, 4-1BB, PD-1, CD80, CD86 and CD83, CD34, CD133, SSEA4, TRA-1-60, EpCAM, or CA9. In some cases, the produced antigen is CD71.

In some cases, the component secretes a signaling chemical that permeates through a wall of the chamber into the interior of the chamber.

In some cases, the loading of the medium into the fluidic device occurs subsequent to the polymerizing of the one or more polymer precursors. In some cases, the loading of the medium into the fluidic device occurs prior to the polymerizing of the one or more polymer precursors. In some cases, the fluidic device comprises one or more channels, and the chamber is located in a channel of the one or more channels.

In another aspect, provided herein is a method of culturing one or more cells, the method comprising: loading a fluidic device with the one or more cells; loading the fluidic device with one or more polymer precursors; polymerizing the one or more polymer precursors to form a chamber within the fluidic device that encloses a cell of the one or more cells, wherein the cell of the one or more cells cannot flow through a wall of the chamber from an interior of the chamber to an exterior of the chamber; loading the fluidic device with a medium containing a component; and after loading the fluidic device with the medium, measuring a characteristic of the cell enclosed in the chamber.

In some cases, the characteristic corresponds to a total number of cells increasing within the chamber through cell division of the cell after the loading of the channel with the medium.

In some cases, the one or more cells comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs), or any combination thereof. In some cases, the one or more cells comprise T-cells. In some cases, the one or more cells comprise OT1 cells.

In some cases, the component is selected from the group consisting of an activator, inhibitor, activated cell, non-activated cell, growth factor, and combinations thereof. In some cases, the component comprises activated OT1 cells, activated T cells, activated B cells, macrophages, dendritic cells, monocytes, astrocytes, microglia, stromal cells, feeder cells, cancer associated fibroblast (CAF), or tumor associated macrophages (TAM). In some cases, the component comprises activated OT1 cells, and the activated OT1 cells cannot pass through the wall of the chamber from the exterior of the chamber to the interior of the chamber. In some cases, the activated OT1 cells secrete a signaling chemical that interacts with the cell within the chamber such that the cell has an increased cell life compared to a situation where there is an absence of the activated OT1 cells proximate to the exterior of the chamber. In some cases, the signaling chemical comprises a growth factor.

In some cases, the characteristic is selected from the group consisting of (i) a change in total number of cells in the chamber during a time duration, (ii) an activation of the cell in the chamber during a time duration, (iii) a change in the total number of live cells in the chamber during a time duration, (iv) a change in the total number of dead cells in the chamber during a time duration, and (v) combinations thereof. In some cases, the characteristic is expression of CD69, CD71, CD25, CD107a, 4-1BB, PD-1, CD80, CD86 and CD83, CD34, CD133, SSEA4, TRA-1-60, EpCAM, or CA9. In some cases, the OT1 cell in the chamber produces a CD71 antigen.

In some cases, the medium comprises non-activated OT1 cells and phorbol myristate acetate (PMA). In some cases, the method further comprises: after the polymerizing of the one or more polymer precursors, measuring a first total number of cells within the chamber; and after a predetermined time period, measuring a second total number of cells within the chamber. In some cases, the loading of the medium into the fluidic device occurs subsequent to the polymerizing of the one or more polymer precursors. In some cases, the loading of the medium into the fluidic device occurs prior to the polymerizing of the one or more polymer precursors. In some cases, the fluidic device comprises one or more channels, and the chamber is located in a channel of the one or more channels.

In another aspect, provided herein is a method of analyzing one or more cells, the method comprising: loading a fluidic device with (i) one or more cells of a first type, (ii) one or more cells of a second type, and (iii) one or more polymer precursors; polymerizing the one or more polymer precursors to form a first chamber within the fluidic device that encloses a cell of the first type of the one or more cells of the first type, wherein the cell of the first type cannot flow through a wall of the first chamber from an interior of the first chamber to an exterior of the first chamber; polymerizing the one or more polymer precursors to form a second chamber within the fluidic device that encloses a cell of the second type of the one or more cells of the second type, wherein the cell of the second type cannot flow through a wall of the second chamber from an interior of the second chamber to an exterior of the second chamber; loading the channel with a medium containing a component; and after loading the channel with the medium, measuring a characteristic of the cell of the first type in the first chamber or the cell of the second type in the second chamber.

In some cases, the first chamber and the second chamber are disposed in a spaced apart arrangement having a separation distance ranging from 5 to 1000 microns. In some cases, the first chamber and the second chamber each have an inner diameter ranging from 10 to 500 microns. In some cases, the second chamber has an inner diameter that is 2 to 10 times greater than an inner diameter of the first chamber.

In another aspect, provided herein is a method of analyzing cells, the method comprising: flowing one or more cells of a first type into a fluidic device, wherein the fluidic device comprises a fluidic structure configured to trap at least a portion of the flowing one or more cells of the first type at the fluidic structure; flowing one or more cells of a second type into the fluidic device along with a medium, wherein the one or more cells of the second type are not trapped by the fluidic structure; and measuring a characteristic of the one or more cells of the first type or the one or more cells of the second type.

In some cases, the fluidic structure is configured to at least partially enclose the one or more cells of the first type. In some cases, the fluidic structure comprises a C-shaped structure to trap the portion of the one or more cells of the first type. In some cases, the fluidic structure comprises an array of pillars configured to trap the portion of the one or more cells of the first type. In some cases, the fluidic device comprises a first surface and second surface. In some cases, in an axis perpendicular to the first surface or the second surface, the fluidic structure does not touch both the first surface and second surface. In some cases, the first surface and the second surface are disposed opposite each other across a channel of the fluidic device.

In some cases, the fluidic structure comprises a non-porous polymer material. In some cases, the fluidic structure comprises a porous hydrogel polymer material configured to partially enclose a cell of the one or more cells of the first type.

In some cases, the one or more cells of the first type comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs), or any combination thereof.

In some cases, the one or more cells of the second type comprise activated OT1 cells, activated T cells, activated B cells, macrophages, dendritic cells, monocytes, astrocytes, microglia, stromal cells, feeder cells, cancer associated fibroblast (CAF), or tumor associated macrophages (TAM), or any combination thereof.

### BRIEF DESCRIPTIONS OF THE DRAWINGS

FIG. 1A diagrammatically illustrates the processing steps in an example of a cell-based therapy, such as, autologous CAR-T cell therapy.

FIG. 1B contains a table listing combinations of assays that may be performed in accordance with the systems and methods described herein.

FIGS. 2A-2F illustrate steps of example assays for determining proliferative capacity of cells in a population followed by their identification by cell surface protein expression and protein secretion.

FIGS. 3A-3B illustrate steps of an assay for determining cytotoxicity of cells of a population. Subsequent assays to identify cells having desirable cytotoxicity values can be carried out as shown in FIGS. 2B-2G.

FIGS. 4A-4B illustrate an assay for determining integrated vector copy number in transduced cells.

FIGS. 4C-4F illustrate an assay for determining integrated vector copy number and insertion site of transduced cells.

FIGS. 4G-4H illustrate an assay for determining single cell genomic copy number variation.

FIGS. 5A-5B diagrammatically illustrate embodiments of systems for carrying out methods described herein.

FIGS. 6A-6B illustrate one embodiment of a flow cell comprising a plurality of channels.

FIGS. 7A-7B illustrate example steps for preparing cDNAs from single cell target templates, such as mRNA, and its sequencing either at the site of a hydrogel chamber (“in situ” or “internal” sequencing) or in an external sequencing instrument after elution from a channel.

FIG. 8A is a schematic illustrating a plurality of hydrogel chambers each containing at least one cell.

FIG. 8B is a schematic illustrating a plurality of hydrogel chambers each containing at least one cell and a plurality activated cells in an interstitial space exterior to the hydrogel chambers.

FIG. 9 is a picture illustrating a plurality of hydrogel chambers each containing at least one cell over a period of time (0 to 48 hours) where there is a plurality activated cells in the interstitial space.

FIG. 10 is a picture illustrating a plurality of hydrogel chambers each containing at least one cell over a period of time (0 to 48 hours) where the cell culture media in the interstitial space contains phorbol myristate acetate (PMA) and a relatively low concentration of un-activated sells.

FIG. 11 is a schematic illustrating a plurality of hydrogel chambers each containing at least one non-activated OT1 cell and at least one dendritic cell where the cell culture media in the interstitial space contains activated cells.

FIGS. 12A and 12B are a set of pictures illustrating a similar experiment where a single hydrogel chamber containing at least two non-activated OT1 cell and at least one dendritic cell that are incubated over a period of time (0 to 24 hours). The pictures are arranged such that the far left hand side column represents a single hydrogel chamber, the middle column represents a magnified view of the single chamber, and the far right side column represents fluorescence imaging using green fluorescent protein (GFP) from the dendritic cell or based on the CD71 activation marker from OT1.

FIG. 13 shows a schematic illustration of a portion of a channel disposed in a fluidic device, according to some embodiments.

FIG. 14 shows a portion of a system as provided herein including an energy source, according to some embodiments.

FIG. 15 shows a computer system that is programmed or otherwise configured to implement methods provided herein.

### DETAILED DESCRIPTION

The practice of the systems and methods described herein may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, cell and tissue culture techniques, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Renault and Duchateau, Editors, Site-directed Insertion of Transgenes (Springer, Heidelberg, 2013); Lutz and Bornscheuer, Editors, Protein Engineering Handbook (Wiley-VCH, 2009); and the like. Guidance for selecting materials and components to carry out particular functions may be found in available treatises and references on scientific instrumentation including, but not limited to, Moore et al, Building Scientific Apparatus, Third Edition (Perseus Books, Cambridge, MA); Hermanson, Bioconjugate Techniques, 3rd Edition (Academic Press, 2013); and like references.

A polymer matrix (e.g., a hydrogel matrix) can be formed adjacent to or around at least of portion of one or more biological components in a fluidic device to isolate selected biological components. A hydrogel matrix may be selectively generated to surround a component. One or more hydrogel or polymer matrix walls can be used to physically separate one or more biological components from one another.

In order to compartmentalize individual components of a biological sample, a polymer matrix (e.g., a hydrogel matrix) can be formed adjacent to or around at least of portion of an individual component in a fluidic device. The hydrogel matrix may be selectively generated to surround a component after the system detects the component or hydrogel matrices can be generated according to a predefined pattern in a fluidic device. The hydrogel matrix may allow reagents and smaller entities to pass while retaining the individual component of the biological sample in place. Because one or more individual components can be localized within a fluidic device (e.g., encapsulated) and the localized components be exposed to one or more reagents and/or washing solutions during and/or in between analyses, multiple assays can be performed within the compartments (e.g., simultaneously, substantially simultaneously, serially, etc.).

Different assays may be performed in different locations of the fluidic device, for example, to test effects of different treatment conditions. By having two or more components within a compartment, interactions between components can be studied as well. The polymer matrix can be degradable “on demand” allowing for controlled localization and release mechanisms. The solutions provided herein can retain spatial information of the components and generate data on a cellular, proteomic, transcriptomic, or genomic level. Since spatial information is retained, the data can be associated (e.g., linked) with phenotypic data. Further, the solutions provided herein can retain spatial information of the components and link data (e.g., phenotypic data) on a cellular, proteomic, transcriptomic, or genomic level.

The present disclosure provides systems for compartmentalizing or isolating one or more biological components. The system can include a fluidic device containing or including one or more biological components. The fluidic device may contain or include one or more polymer precursors. In some cases, the fluidic device can comprise a first surface configured to couple or receive at least one of the one or more biological components to form a coupled biological component. The systems may also include at least one energy source, wherein the energy source is in communication with the fluidic device. In some embodiments, the energy source may be in optical communication with the fluidic device. In various embodiments, the at least one energy source may form a polymer matrix on or adjacent to at least a portion of the one or more biological components.

In some cases, a sample may be introduced or provided to the system. In certain cases, the sample may comprise one or more biological components. In various cases, the biological components may be physically separated. In some cases, the biological components may be physically separated but in fluidic communication with one another. In certain cases, the biological components may be in chemical communication with one another. The system may be used for single-cell analysis. In some embodiments, the system may be used for single-cell analysis on a genome level. For example, the system may be used for genome sequencing. For another example, the system may be used for deoxyribonucleic acid (DNA) sequencing. The system may be used for DNA sequencing of cell-free DNA, whole genome sequencing, whole exome sequencing, targeted sequencing, or 16S sequencing. The system may be used for studying DNA tags attached to biomolecules of interest. The biomolecules may comprise proteins, metabolites, etc. In some cases, the DNA may be a nuclear DNA or a mitochondrial DNA. The system may be used for single-cell or bulk analysis on a transcriptome level. For example, the system may be used for ribonucleic acid (RNA) sequencing. For example, the system may be used for 3′ or 5′ gene expression analysis, immune repertoire study of a cell, or full-length mRNA analysis. In some embodiments, the system may be used for single-cell analysis on a proteome level. The system may be used for functional assay(s) of a biological component. The system may be used for studying surface proteins, secreted proteins, or metabolites of a biological component. In some cases, the system may be used to measure a quality of a biological component. In some cases, the measured quality may be the size or shape of a biological component. In some cases, the system may be used to study epigenomics, DNA methylation, or chromatin accessibility in a biological component. The system may be used for other suitable assays, experiments, and processes.

In certain embodiments, the system may be used for single-cell analysis on an indirect cell-cell interaction level. For example, an effect of one or more molecules produced from a first cell on a second cell can be analyzed using the system as provided herein. In various embodiments, the system may be used for analyzing direct cell-cell interactions. For example, two or more cells (e.g., a first cell and a second cell) can be in physical contact and the effect or effects of the first cell on the second cell, or vice versa, can be analyzed using the system as disclosed herein. In some embodiments, the system may be used for drug response analysis in a biological component. In certain embodiments, the system may be used for analyzing a biological component's response to various physiological conditions (e.g., various media, temperature, mechanical stimuli, etc.). In some embodiments the analyte is selected from a plurality of analytes introduced into the fluidic device.

In certain embodiments, one or more polymer precursors may be added to or included with the biological sample. One or more biological samples and one or more polymer precursors may be introduced into the system (e.g., into the fluidic device of the system). The one or more biological samples and the one or more polymer precursors may be introduced into the fluidic device in any order (e.g., in parallel, sequentially, etc.). For example, the biological sample(s) may be introduced prior to the polymer precursor(s), the polymer precursor(s) may be introduced prior to the biological sample(s), the biological sample(s) and polymer precursor(s) may be introduced simultaneously (or substantially simultaneously), or in any other suitable manner or order. In some embodiments, a polymer precursor may include one or more hydrogel precursors, a porogen, and a photoinitiator.

As used herein, the term “photoinitiator” can denote a species that generates a radical upon photoexcitation. In many cases, a photoinitiator included in a polymer precursor formulation is a type I photoinitiator, that is a molecule that generates radicals through intramolecular cleavage (e.g., homolysis) upon photoexcitation, or a type II photoinitiator, that is a molecule that abstract an electron or hydrogen atom from a co-initiator following photoexcitation. Examples of a photoinitiator includes one of lithium phenyl-2,4,6-trimethylbenzoylphosphinate (LAP), Irgacure 2959, diphenyl(2,4,6-trimethylbenzoyl)phosphine oxide (TPO) nanoparticles, 2,2′-azobis [2-methyl-N-(2-hydroxyethyl) promionamide] (VA-086), BAPO-Oli, BAPO-Ona, Eosin-Y, Riboflavin, acetophenone, anisoin, anthraquinone, anthraquinone-2-sulfonic acid, benzil, benzoin, benzophenone, 3,3′,4,4′-benzophenonetetracarboxylic dianydride, 4-benzoylbiphenyl, 2-benzyl-2-(dimethylamino)-4′-morpholinobutyrophenone, dibenzosuberenone, 2,2-diethoxyacetophenone, 2-ethylanthraquinone, ferrocene, 2-isopropylthioxanthone, methyl-2-benzoylbenzoate, and thiooxanthen-9-one, and combinations thereof.

As used herein, the term “porogen” can denote a species that modulates the porosity of a polymer matrix but does not incorporate into the polymer matrix during polymerization. Porogens typically diffuse out of polymer matrices following polymerization, leaving pores in the regions that they occupied. Porogen size, concentration, hydrophobicity, and hydrophilicity can thus influence pore density and pore size in polymer matrices. Examples of a porogen can be polyethylene glycol (PEG, molecular weight from 1 kDa to 1000 kDa), 8 arm PEG, 4 arm PEG, 3 arm PEG, particles (e.g., polymeric, ceramic, metal, metal oxide, or hydrogel particles), alginate, vesicles such as liposomes or micelles, and combinations thereof.

The one or more polymer precursors may be stored and/or introduced separately into the system. In some cases, the one or more polymer precursors may be mixed with the one or more biological components prior to introduction into the system. In various cases, the one or more polymer precursors may be mixed with the one or more biological components after introduction into the system.

The system may comprise a fluidic device. In some embodiments, the fluidic device may include one or more polymer precursors. In other words, one or more polymer precursors may be disposed within at least a portion of the fluidic device (e.g., within at least a portion of a channel of the fluidic device). In some embodiments, the fluidic device may comprise one or more channels or chambers. In some embodiments, the fluidic device may include at least 1, 2, 3, 4, 5, 6, 7, 8, 9, 10, 15, 20, 30, 40, 50, 100, 200, 300, 400, 500, 600, 700, 1,000, 10,000 channels or chambers, or any number of channels or chambers between any of the two numbers mentioned herein. In some embodiments, the fluidic device comprises more than 10,000 channels or chambers. As described herein, the fluidic device may include one or more channels. The fluidic device may also, or alternatively, include one or more chambers. The terms channel and chamber may be used interchangeably in the disclosure herein unless indicated otherwise. For example, a channel or a chamber of the fluidic device may comprise a first surface, a second surface, or more surfaces.

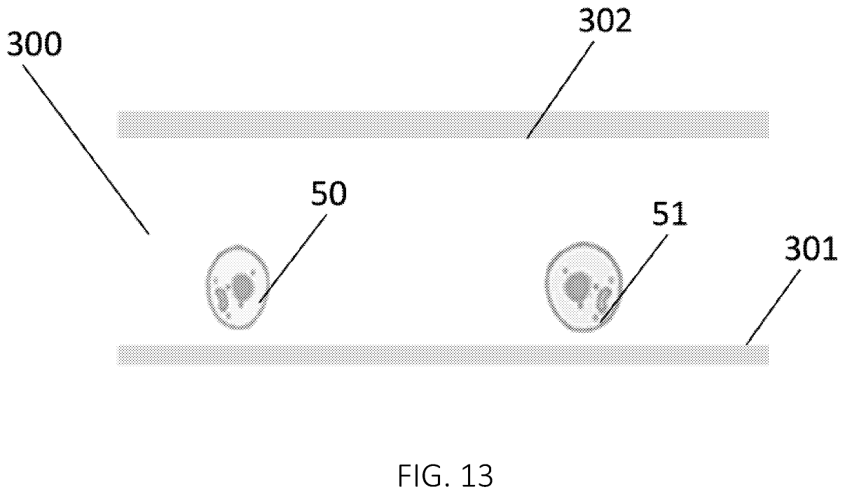

A channel or chamber of a fluidic device (also sometimes referred to as a “flow chamber,” “flow channel,” or “reaction chamber,” as opposed to a chamber that is formed from polymer matrix walls within a channel) may receive or be configured to receive a biological sample. FIG. 13 shows a simplified schematic cross-sectional side view illustration of a portion of a channel 300 that may be disposed in at least a portion of a fluidic device of a system as provided herein. The fluidic device may comprise a channel 300. The channel 300 may comprise a first surface 301. Further, the channel 300 may comprise a second surface 302. In some embodiments, the first surface 301 and the second surface 302 are disposed, placed, or positioned opposite of one another (e.g., as depicted in FIG. 13). In some embodiments, a middle spacer layer of double sided adhesive with a cut-out portion can be used to position the first surface 301 and second surface 302 in a facing relationship to at least partly form the flow channel. In some embodiments, the first surface and second surface are substantially parallel, so that the perpendicular distance between them is substantially the same throughout the channel, for example, where chambers are formed. In some embodiments, the perpendicular distance between a first surface and a second surface depends in part and the nature and size of the biological components to be analyzed. In some embodiments, such as, those adapted to analyzing mammalian cells, the perpendicular distance between a first surface and a second surface may be in the range of from 10 μm to 500 μm, or in the range of from 50 μm to 250 μm. In some embodiments, the perpendicular distance between a first surface and a second surface may be in the range of from twice the average size of the biological component to be analyzed to five times the average size of the biological component to be analyzed. In some embodiments, the perpendicular distance between a first surface and a second surface may be in the range of from twice the average size of the largest biological component in the biological sample to five times the average size of the largest biological component in the biological sample. In some embodiments, the first surface 301 may be a lower surface. In certain embodiments, the second surface 302 may be an upper surface. The channel 300 may receive a biological sample comprising one or more biological components 50, 51. The channel 300 may receive one or more polymer precursors. As illustrated in FIG. 13, the biological components 50, 51 may include cells. However, as discussed herein, the biological components may include tissues, proteins, nucleic acids, etc. In some embodiments, the first surface 301, the second surface 302, or both surfaces may couple or receive, or be configured to couple or receive, at least one of the one or more biological components 50, 51. In some cases, the first surface 301 may couple or receive, or be configured to couple or receive, a biological component (e.g., biological components 50, 51). In certain cases, the second surface, 302 may couple or receive, or be configured to couple or receive, a biological component (e.g., biological components 50, 51). In some embodiments, the first surface and/or second surface can be optically transmissive so that visible and UV light can transmit through one or both of the surface for the generation of polymeric hydrogels, imaging of the flowcell, and the measurement of the analyte and biological components.

In certain cases, a channel may have a cross-sectional area that is rectangular, circular, semi-circular, or oval. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

In some cases, the first surface 301, the second surface 302, or both surfaces 301, 302 may be functionalized, for example, with a coating (e.g., a surface coating). In some embodiments, the surface coating may be a surface polymer. Some non-limiting examples of surface coatings may include a capture reagent (e.g., pyridinecarboxaldehyde (PCA)), a functional group to capture one or more moieties (e.g., a chemical moiety), an acrylamide, an agarose, a biotin, a streptavidin, a strep-tag II, a linker, a functional group comprising an aldehyde, a phosphate, a silicate, an ester, an acid, an amide, an alkyne, an azide, an aldehyde dithiolane, or a combination thereof. In various embodiments, the surface coating may include a functional group to capture one or more moieties. For example, the acrylamide, the agarose, etc. may include such a functional group. In certain embodiments, the surface polymer may comprise polyethylene glycol (PEG), a thiol, an alkene, an alkyne, an azide, or combinations thereof. In various embodiments, the surface polymer may comprise a silane polymer. In some embodiments, the surface polymer may be functionalized with at least one of an oligonucleotide, an antibody, a cytokine, a chemokine, a protein, an antibody derivative, an antibody fragment, a carbohydrate, a toxin, or an aptamer.

In some cases, the first surface 301, the second surface 302, or both surfaces 301, 302 may comprise one or more barcodes (e.g., nucleic acid barcodes). In some embodiments, the first surface 301, the second surface 302, or both surfaces 301, 302 may comprise 1, 2, 3, 4, 5, 6, 7, 8, 9, 10, 15, 20, 30, 40, 50, 100, 200, 300, 400, 500, 600, 700, 1,000, 10,000, 50,000, 100,000, 250,000, 500,000, 1,000,000, 2,000,000, 5,000,000, 10,000,000, 15,000,000 barcodes, or any number of barcodes between any of the two numbers mentioned herein. The barcodes may cover an area of about 500 nm² to about 100,000 μm² and preferably 500 nm² to about 5000 μm². In some embodiments, the first surface 301, the second surface 302, or both surfaces 301, 302 may comprise at most about 10,000,000 total number of barcodes. The barcodes may be different from one another (e.g., each barcode may be unique). In certain embodiments, a first portion or subset of the barcodes may be different from a second portion or subset of the barcodes. There may be 2, 3, 4, 5, 10, 15, 20, 25, 50, 75, 100, 1,000, 10,000 portions or subsets of the barcodes, or any number of portions or subsets of the barcodes between any of the two numbers mentioned herein. In some cases, a barcode (or a portion/subset of barcodes) may be associated with the location of the barcode on a surface (location coordinates (e.g., x-, y-coordinates) on a surface of a channel). A barcode may be attached to or coupled to the captured biological component. In some embodiments, the barcode may be a unique identifier that distinguishes a biological component from other biological components (e.g., that identifies a first biological component versus a second biological component). In some embodiments, a barcode may comprise a nucleic acid sequence (e.g., common sequence) to capture a biological component, or used in amplification. In some embodiments, a barcode may comprise a unique identifier comprising a unique nucleic acid sequence (e.g., DNA sequence, RNA sequence, etc.), protein tag, antibody, or an aptamer. In some embodiments the barcode may comprise a fluorescent molecule. In some embodiments, a location of the captured biological component may be associated with the unique identifier to, for example, retain spatial information of a biological component.

In some embodiments, the fluidic device may be a flow cell. For example, the fluidic device may be used for sequencing (e.g., DNA or RNA sequencing). In some embodiments, the fluidic device may be a microfluidic device. In certain embodiments, the fluidic device may be a nanofluidic device.

The system disclosed herein may comprise one or more energy sources. The energy source may be in communication with the fluidic device. In some embodiments, the energy source may be in optical communication with the fluidic device. In some cases, the energy source can be used to form one or more polymer matrices in the fluidic device (e.g., on or adjacent to a surface of a channel or chamber of the fluidic device). In some embodiments, the energy source may comprise a light generating device, a heat generating device, an electrochemical reaction generating device, an electrode, or a microwave device. A polymer matrix may be formed in a channel of the fluidic device. The energy source may direct or transfer energy to a predetermined position in the fluidic device. The energy may cause or activate the one or more polymer precursors to form a polymer matrix (e.g., to polymerize) in the predetermined position.

In some embodiments, the polymer matrix may comprise a hydrogel. In some embodiments, the hydrogel may be porous enough, or have pores of a suitable size, to allow movement or transfer of a reagent (e.g., an enzyme, a chemical compound, a small molecule, an antibody, etc.) through the polymer matrix, while the hydrogel may not allow movement or transfer of the biological component (e.g., DNA, RNA, a protein, a cell, etc.) through the polymer matrix. In some embodiments, the pores may have a diameter from 5 nm to 100 nm. In some embodiments, the pores may have a diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have a diameter larger than 100 nm. In some embodiments, the pores may have a diameter smaller than 5 nm. The reagent may comprise an enzyme or a primer having a size of less than 50 base pairs (bp). A primer may comprise a single-stranded DNA (ssDNA). In some embodiments, a primer may have a size from 5 bp to 50 bp. In some embodiments, a primer may have a size from 5 bp to 10 bp, 10 bp to 20 bp, from 20 bp to 30 bp, 30 bp to 40 bp, or 40 bp to 50 bp. In some embodiments, a primer may have a size of more than 50 bp. In certain cases, a primer may have a size of less than 5 bp. A reagent may comprise a lysozyme, a proteinase K, hexamers (e.g., random hexamers), a polymerase, a transposase, a ligase, a catalyzing enzyme, a deoxyribonuclease, a deoxyribonuclease inhibitor, a ribonuclease, a ribonuclease inhibitor, DNA oligos, deoxynucleotide triphosphates, buffers, detergents, salts, divalent cations, or any other suitable reagent.

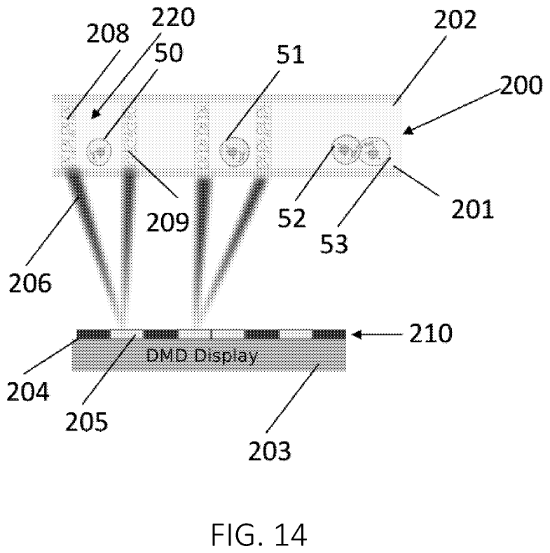

FIG. 14 shows a portion of a system as provided herein including an energy source 203. The embodiment of FIG. 14 may include components that resemble components of FIG. 13 in some respects. For example, the embodiment of FIG. 14 includes a channel 200 that may resemble the channel 300 of FIG. 13. With continued reference to FIG. 14, the channel 200 of the system may include a first surface 201 and a second surface 202. In some embodiments, the energy source 203 may comprise one or more energy emitting portions (e.g., an energy emitting portion 205). In some embodiments, the energy source 203 may comprise an energy one or more non-emitting portions (e.g., a non-emitting portion 204). The non-emitting portion 204 may not emit, or be configured to emit, energy. In some embodiments, the emitting portion 205 can emit energy in the form of electromagnetic waves (e.g., microwaves, light, heat, etc.) to at least a portion of the fluidic device. In certain embodiments, the emitting portion 205 can emit energy to the fluidic device. In some embodiments, the fluidic channel may be coupled to on a movable stage. In other embodiments, light may be projected to or onto at least a portion of the fluidic channel to generate one or more polymer matrices. The light may be directed to various parts of the fluidic channel. In some embodiments, the emitting portion 205 may be coupled to an objective (e.g., a microscope objective or lens), where the objective may be moved to different portions of the fluidic device. The objective may provide a shape (e.g., virtual mask) to allow light to form a pattern on the fluidic device, in order to form a polymer matrix similar or complementary to the pattern. In various embodiments, the one or more polymer precursors in the fluidic device or mixed with the biological sample can absorb emitted energy 206. In some embodiments, the emitted energy 206 can form, or be sufficient to form, a polymer matrix from the one or more polymer precursors. For example, a portion of the one or more polymer precursors within the channel 200 of the fluidic device may be activated by the emitted energy and a polymerization reaction may be initiated to form a polymer matrix.

The energy source (e.g., light source) may be coupled to the fluidic device via an objective (e.g., a microscope objective or lens). The energy source may be directed to a portion of the fluidic channel (e.g., via a movable objective). In some cases, the light source, the objective, and/or the fluidic channel are movable to allow emission of energy to the fluidic channel so as to generate a pattern on at least a portion of a surface of the fluidic device. The polymer matrix may be formed similarly or complementary to the pattern of energy emission.

In some embodiments, a first polymer matrix 208 can be formed on or adjacent to a biological component 50. In certain embodiments, the first polymer matrix 208 can form a cylindrical analysis chamber or compartment 220 that separates (e.g., physically separates) the biological component 50 from other biological components (e.g., biological components 51, 52, or 53) in the fluidic device. Stated another way, the polymer matrix may compartmentalize the channel (e.g., channel 200) in cooperation with the first surface 201 and the second surface 202. In various embodiments, the polymer matrix may partially surround a biological component. For example, a polymer structure fully surrounding a biological component may form a closed structure (e.g., a hollow cylinder-shaped polymeric structure) or a partially open structure (e.g., a crescent-shaped polymeric structure). In some embodiments, two or more polymer matrices may be formed adjacent to a biological component forming a compartment separating the biological component from other biological components. In certain embodiments, the polymer matrix may comprise or form a wall (e.g., a polymer matrix wall).

With continued reference to FIG. 14, in some cases, the energy source 203 can, or be configured to, form or produce one or more emitting portions 205 and one or more non-emitting portions 204. The systems disclosed herein may further include a spatial energy modulating element to direct energy from the energy source to one or more targeted portions of the fluidic device. For example, the spatial energy modulating element may be configured to selectively direct the energy from the energy source to form a polymer matrix in a discrete area of the fluidic device. In some embodiments, the discrete area is chosen based on the location of a biological component. In some embodiments, the area of the discrete area is less than the area of the fluidic device. In some embodiments, a biological component is captured within the discrete area. In some embodiments, the size and shape of the discrete area is adjustable according to the size, shape, or other properties of the biological component. In some embodiments, an algorithm is used to determine the shape and size of the discrete area. In some embodiments, the algorithm is a supervised, a self-supervised, or an unsupervised learning algorithm. The spatial energy modulating element may be configured to selectively direct the energy by, for example, inhibiting or preventing energy from being directed to one or more portions other than the one or more targeted portions of the fluidic device. In some embodiments, the spatial energy modulating element may comprise a physical mask. In some cases, the spatial energy modulating element may comprise a virtual mask. In some cases, the spatial energy modulating element may be a spatial light modulator (SLM). In some embodiments, the SLM is a digital micromirror device (DMD). In some embodiments, the SLM is a laser beam steered using a galvanometer. In some embodiments, the SLM is liquid-crystal based.

In some embodiments, the first surface 201 or the second surface 202 may comprise a detector that detects, or is configured to detect, one or more locations of one or more biological components in the fluidic device (e.g., in the channel 200). In certain embodiments, the energy source 203 can comprise, be coupled to, or be in communication with a detector that detects, or is configured to detect, a location of a biological component in the fluidic device. In some embodiments, the detector may be a microscope objective for imaging the fluidic device. In various embodiments, a mask may be generated using an image obtained from at least a portion of the fluidic device. The mask may allow or permit the energy source 203 to emitting energy in or toward one or more locations or positions where one or more biological components are present on or adjacent the first surface 201. The mask may inhibit or prevent the energy source 203 from emitting energy in or toward one or more locations or positions where one or more biological components are present on or adjacent the first surface 201. In some embodiments, the image may be obtained from a camera (e.g., a digital camera, fluorescent imaging camera, etc.). In some embodiments, the imaging is bright-field imaging, phase-contrast imaging, or fluorescence imaging, or any combination thereof. In some embodiments, the camera may be coupled to, connected to, or in communication with the energy source 203. For example, the camera (not shown) may be in electrical communication with the energy source 203. In some embodiments, the energy source 203 may comprise the camera. In various embodiments, the energy source 203 may comprise a microscope (e.g., a fluorescence microscope, a confocal microscope, lens-free imaging system, a transmission electron microscopy (TEM), a scanning electron microscope (SEM), etc.). The microscope may be used to detect one or more positions of one or more biological components (e.g., in combination with the detector).

In some embodiments, an algorithm is used to determine where a biological component or analyte is located based on the imaging. In some embodiments, the algorithm is a supervised, a self-supervised, or an unsupervised learning algorithm. In some embodiments, the objective is coupled to an energy source to emit energy to the predetermined portion in the fluidic channel.

The systems and methods described herein are directed to methods and systems for carrying out one or more large-scale multiplexed assays that assess concurrently multiple characteristics of cells, especially for the purpose of developing, and assuring the quality of, cell-based therapeutics. In some embodiments, the systems and methods described herein comprise carrying out concurrently a plurality of assays to determine multiple cellular characteristics important for cell-based therapies including, but not limited to, cell-based therapies for treating cancers, e.g. using CAR-T cells, or cell-based therapies for treating other conditions, such as, spinal injury, stroke, diabetes, e.g. using induced pluripotent stem cells, mesenchymal stem cells, embryonic stem cells, or the like. In some embodiments, the systems and methods described herein are directed to systems for carrying out concurrently a plurality of single cell assays designed to detect or measure selected or predetermined cell characteristics of populations of cells. In some embodiments, one or more of such cellular assays depend on or utilize hydrogel chambers (sometimes referred to herein as “analysis chambers,” or simply “chambers,” or “hydrogel cages”). In some embodiments, such cellular assays are single cell assays so that one or more hydrogel chambers employed in such assays each enclose only a single cell. In some embodiments, such single cell assays may comprise a single engineered cell and one or more target cells, that is, one or more cells that may be acted on (e.g. killed) by the engineered cell.

“Cells” that may be assayed by methods and systems described herein may comprise any biological cells including, but not limited to, vertebrate, non-vertebrate, eukaryotic, mammalian, microbial, protozoan, prokaryotic, bacterial, insect, or fungal cells. In some embodiments, mammalian cells are assayed by methods and systems described herein. In particular, any population of mammalian cells which may be, or have been, induced, treated, modified or genetically altered (i.e. genetically engineered) for use in a medical, industrial, environmental, or remedial process, may be analyzed by methods and systems described herein. In some embodiments, “cells” as used herein comprise genetically modified mammalian cells. In some embodiments, “cells” comprise stem cells that have been induced to differentiate. In some embodiments, “cells” refer to cells modified by CRISPR Cas9 techniques. In some embodiments, “cells” refer to cells of the immune system including, but not limited to, cytotoxic T lymphocytes, regulatory T cells, CD4+ T cells, CD8+ T cells, natural killer cells, antigen-presenting cells, or dendritic cells. Of special interest are cytotoxic T lymphocytes engineered for therapeutic applications, such as cancer therapy.

As used herein, the term “assay” refers to a process for detecting or measuring a cellular characteristic or property of single cells or of a population of cells. Typically process steps of an assay comprise a chemical, biochemical or molecular reaction (such as a cleavage of a bond, specific binding of complementary components, enzyme reactions, dissolution of complementary components, or the like) or a change of physical state (such as an increase or decrease in temperature, change in energy level, or the like) and result in the generation of a signal (or signals) from which the presence, absence or magnitude of a quantity related to a cell may be inferred. The nature of the signal produced by an assay may vary widely and can include, but is not limited to, an electrical signal, an optical signal, a chemical signal, or a material signal. A material signal comprises the production of a material that comprises information that can be extracted. For example, a material signal may be the amplification of a polynucleotide whose length, quantity, composition, or nucleotide sequence is indicative of a cellular characteristic. For example, a barcode oligonucleotide may be a material signal. Characteristics or properties of cells that are detected or measured may vary widely and include, but are not limited to, cytotoxicity, viability, proliferation capacity under selected conditions, size, shape, motility, types and profiles of cell surface, or cell membrane proteins, types and profiles of secreted proteins, production of metabolites, transcriptome, gene copy numbers, gene or allele identity, chromatin accessibility profiles, vector copy numbers for engineered or infected cells, and the like. Assays of special interest for cell-based therapy include, but are not limited to, measurement of characteristics listed in Table 1.

TABLE 1
Assays

| Assay | Comments |
|---|---|
| Cytotoxicity | Ability to kill target cells |
| Viability | Fraction of dead cells |
| Proliferation rate | Rate of “expansion” or population growth, especially of |
| | desired cell type |
| Phenotype | For example, based on cell surface markers, secreted |
| | proteins, structural features (e.g. as determined from |
| | images), and the like |
| Cellular morphology | For example, as determined from images |
| Identity | Confirmation that cells are the from patient (autologous) |
| | or from the same donor (allogenic) |
| Purity | For example, fraction of total population of cells that |
| | consist of the desired type (e.g. CD8+ T cells); absence |
| | of contaminants, e.g. mycoplasma, fungus, bacteria, etc. |
| Activation | Proliferation and differentiation characteristic of an |
| | immune response, usually after specific treatment to |
| | stimulate such response |
| Transduction efficiency | For example, fraction of cells of the desired type that |
| | incorporate at least one vector (e.g. encoding a CAR or |
| | other feature, either epigenetically or by integration into |
| | the cell genome) |
| Vector copy number | Number of separate integrations of a vector into a |
| | therapeutic cell's genome |
| Integration site analysis | Positions in therapeutic cell's genome where vector has |
| | integrated |
| Chromatin accessibility | For example, to determine whether transposase- |
| profile | accessible chromatin falls within expected ranges, e.g. |
| | both in regard to magnitude and specific locations |
| Transcriptome | Gene expression profile |
| Epigenetic profile | For example, for transiently transfected cells |
| Suicide gene function | For example, to kill and eliminate therapeutic cells in |
| | the event of deleterious side effects |
| Genetic stability | Measurement of genomic copy number variation |
| Replicative competent | Measure presence or absence of viral replication |
| retrovirus | components in, for example, a transduced therapeutic |
| | cells |
| Dose | For example, for CAR T-cells, number of viable T cells |
| | expressing CAR (may be expressed as either a total |
| | number, a concentration or a number per kg of patient |
| | body weight) |

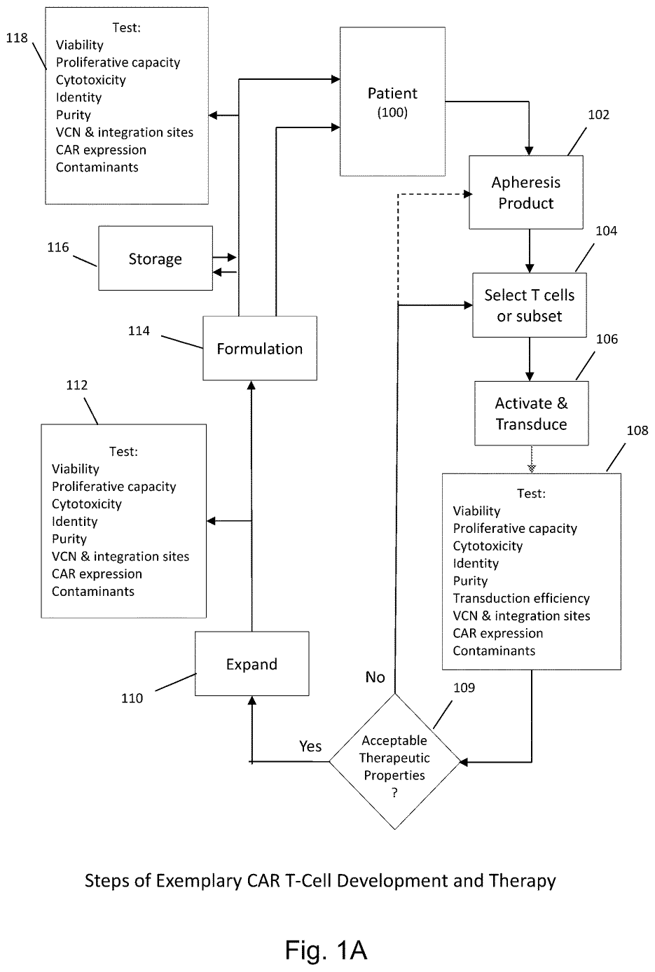

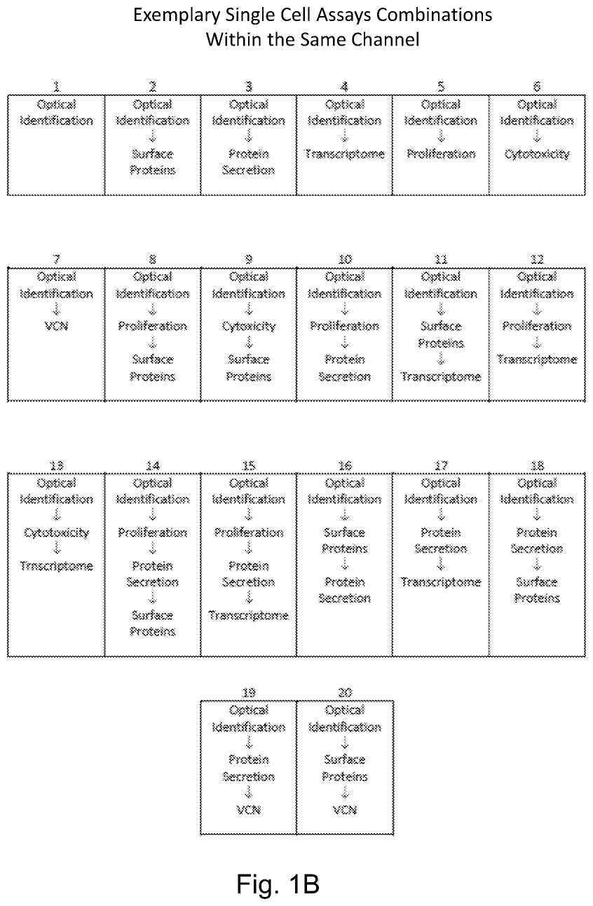

As mentioned above, CAR-T cell therapy, e.g. to treat cancers, is an example application of the systems and methods described herein. The major steps of autologous CAR-T cell production for therapy are illustrated in FIG. 1A, and are described in many publications, for example, Wang et al, Molecular Therapeutics-Oncolytics, 3: 16015 (2016); Barrett et al, Annu. Rev. Med., 65:333-347 (2014). The figure illustrates where in the production process quality assurance testing may take place. White blood cells containing lymphocytes are separated from other blood components of patient (100) by apheresis (102), e.g. Allen et al, Transfusion, 57 (5): 1133-1141 (2017). Further enrichment (104) of particular subsets of lymphocytes may be carried out by depleting undesired subsets or enriching desired subsets using magnetic beads, or other techniques, e.g. to remove T regulatory cells which may suppress desired activity of the CAR-T cells. After a suitable subset is selected, T cells are activated and transduced (106); that is, they are made competent to perform immune functions and to express CARs. Activation may be accomplished by a variety of treatments, e.g. exposure to anti-CD3/anti-CD28 beads. Transduction may also be accomplished by a variety of techniques, e.g. lentivirus transduction and integration into T cell genomes, e.g. Wang et al (cited above); Barrett et al (cited above). After such transformations, T cells are tested (108), for example, using the methods and systems described herein, to determine whether desired properties for CAR-T cell therapy are present, such as, an adequate degree of cytotoxicity towards desired target cells, viability, proliferative capacity, identity, absence of contaminants, non-excessive virus copy number values, and so on. If the activated and transduced cells do not have acceptable therapeutic properties (109), then new cells must be prepared. If they have acceptable therapeutic properties, then the cells are expanded (110); that is, they are cultured to increase their numbers. As conditions for expansion may alter the cells, they are tested again for desirable properties after expansion (112). Successfully expanded cells may be formulated (114) for infusing into a patient (100) and/or storage (116) for subsequent infusions. If subsequence infusions are required, cells removed from storage are again tested to assure that they possess the desired therapeutic properties. Example single cell assays that may be carried out with methods and systems described herein are listed in Table 1 and FIG. 1B. In some embodiments, successive assays each acquiring additional information may be conducted on the same cell, or cells, in chambers of a channel.

CAR-T cell or therapeutic stem cell development and manufacture is an example application of the systems and methods described herein. In some embodiments, methods for determining one or more cellular characteristics, such as those relevant to CAR-T cell or therapeutic stem cell development or production, may be performed by the following steps: (a) providing a fluidic device comprising (i) one or more channels each comprising a first surface and, optionally, assay components for detecting or measuring a cellular characteristic, (ii) a spatial energy modulating element in optical communication with each first surface, and (iii) a detector that identifies positions of cells in each channel based on one or more optical signals therefrom; (b) disposing cells on the first surface of the channel; (c) loading each channel with one or more polymer precursors; (d) synthesizing one or more chambers in the channels, each chamber enclosing a cell, by projecting light into each channel with the spatial energy modulating element such that the projected light causes cross-linking of the one or more polymer precursors to form polymer matrix walls of the chambers, wherein the positions of the synthesized chambers are determined in each channel by the positions of the cells enclosed or encased thereby identified by the detector; and (e) incubating the cells under assay conditions in each channel to generate signals from chambers of each channel indicative of cellular characteristics. In some embodiments, incubating the cells under assay conditions comprises performing one or more assays on the cells in the channels to determine one or more of the cellular characteristics selected from the group consisting of cytotoxicity, viability, proliferation rate, phenotype, vector copy number, vector integration sites, transcriptome, and genomic copy number variation. In some embodiments, polymer matrix walls of chambers comprise degradable hydrogels. In some embodiments, incubating comprises lysing the cells and capturing on the first surface one or more cellular components by capture agents. In some embodiments, incubating further comprises degrading the degradable polymer matrix walls after such one or more cellular components are captured. As used herein, the terms “capture element” and “capture agent” are used interchangeably. In some embodiments, capture agents (or capture elements) are covalently attached to a surface of a channel. In some embodiments, capture agents (or elements) comprise oligonucleotides having sequences capable of hybridizing to a cellular component, either directly, e.g. polyA tail of mRNA, or indirectly, e.g. an adaptor strand of an adaptored genomic DNA fragment produced by tagmentation. In some embodiments, capture agents (or elements) comprise one or more antibodies. In some embodiments, capture agents (or elements) comprise spatial barcodes. In some embodiments, capture agents (or elements) comprising oligonucleotides further comprise primers for solid phase amplification, such as, bridge PCR. In some embodiments, a fluidics device comprises a plurality of channels. In some embodiments, a plurality of cells is disposed on a surface of each channel. In some embodiments, such plurality of cells comprises 100 or more cells, or 1000 or more cells, or 10,000 or more cells, or a number of cells within the range of from 100 to 10⁶ cells.

In some embodiments, the steps of disposing cells and loading polymer precursors are performed by mixing the cells and polymer precursors outside of the fluidic device then loading the channel with the mixture of cells and polymer precursors. In some embodiments, cells are delivered to a first surface in a fluid where they are randomly dispersed over the first surface. In some embodiments, after cells are delivered to a surface of a channel they are randomly disposed on the surface. In some embodiments, the step of incubating under assay conditions may comprise only a single step of an assay comprising more than one step, such as, for example, a step of detecting a signal (or in the case of a material signal, generating a sequencing-ready nucleic acid), or such step of incubating under assay conditions may comprise a plurality of steps of a multi-step assay. In some embodiments, cells may be treated or subjected to assay steps prior to loading into a channel of a fluidic device, so that the step of incubating may comprise only a single step of a multi-step assay, such as signal generation and/or signal collection. In other embodiments, the step of incubating may comprise the implementation of an assay step or part of an assay, such as, cDNA synthesis, second strand synthesis, capture of an assay component or a cellular component, or the like. In some embodiments, assay conditions may comprise a series of steps each with different conditions (e.g. temperature, pH, presence or absence of particular reagents, such as, primers or an enzyme, e.g., a ligase, a polymerase, a transposase, or the like). For example, such steps may comprise loading primers so that they diffuse across chamber walls and anneal to target sites on a template strand in a sample in the chamber, extending the annealed primers, heating the channel so that the extended primers melt from its template strand, capturing the melted extended primers by capture agents attached to the first surface, and so on.

In some embodiments, cells are adherent with respect to a first surface so that upon settling and contact the cells remain on the surface, even in the presence of fluid movement. In other embodiments, cells are non-adherent with respect to a first surface so that although they may settle on the first surface they may move or return to a suspension in the presence of fluid movement. One of ordinary skill in the art would recognize that a first surface may be treated (for example, by the attachment of appropriate capture elements) to retain non-adherent cells, either over the entire first surface (or polymer matrix walls or second surface) or selected portions thereof. In some embodiments, assay components that may be provided with or as part of a channel include, but are not limited to, capture elements, such as, capture oligonucleotides, primers for surface amplification, antibodies, functional groups that may react with hydrogel components, beads, and the like. In some embodiments, such assay components may be attached to any one of a first surface, a second surface or a polymer matrix wall exclusively, or on combinations of such surfaces, either exclusively, or in combination with other reagents. In some embodiments, assay components that may be provided after synthesizing gel chambers include, but are not limited to, lysing reagents, transcription reagents, reverse transcription reagents, antibodies, polymerases, primers, beads, and the like. As used herein, “channel” means a container capable of holding fluid (which may be static or flowing) and having at least one surface on which cellular assays may be conducted. In some embodiments, a channel may have a first surface and/or a second surface on which chambers may be synthesized and/or on which cellular or assay components may be attached. In addition, in some embodiments, cellular or assay components may be attached or capture by capture elements on a polymer matrix wall. As used herein, reference to a “first surface” (for example, as a surface comprising capture elements) may comprise a second surface, or as appropriate, a polymer matrix wall. As used herein, reference to a “surface” without reference to “first” or “second” is intended to comprise a first surface or a second surface. In some embodiments, a channel may constrain a flow of fluid therethrough from an inlet to an outlet. In other embodiments, a channel may comprise a non-flowing volume of fluid that may be removed, replaced or added to by way of an opening or inlet; that is, in some embodiments, a channel may be a well or a well-like structure, such as a trough. In some embodiments, hydrogel chambers are synthesized; and in other embodiments degradable hydrogel chambers are synthesized. In some embodiments, particularly for CAR-T cells, a plurality of cellular characteristics may be selected from the following characteristics: cytotoxicity, proliferative capacity or proliferation rate, activation status, cellular identity, purity, gene expression profile or transcriptome, epigenetic profile, sequence copy number (e.g., integrated viral copy number for transduced cells, plasmid copy number for transiently transfected cells, gene copy number, or the like). Additional assays may include culture contamination assays including, but not limited to, viral, bacterial, yeast, mold, or mycoplasma assays, endotoxin assays, and cellular morphology assays.

Another example application of the systems and methods described herein includes screening CRISPR/Cas9-based modified cell populations. For example, Shifrut et al (Cell, 175 (7): 1958-1971 (2018)) screened populations of T cells transduced by lentivirus producing single guide RNAs of a library targeting known T cells genes. The objective was to identify genes which when knocked out enhanced the T cell response, which was measured by growth rate. The systems and methods described herein are perfectly suited to provide single cell proliferation rates on a large scale such as required by such studies.

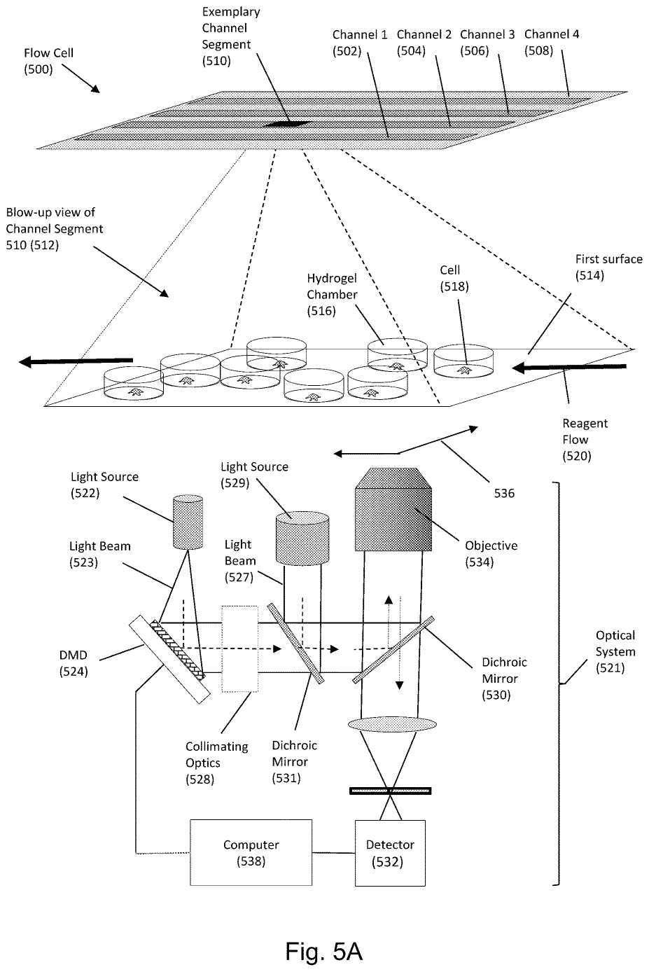

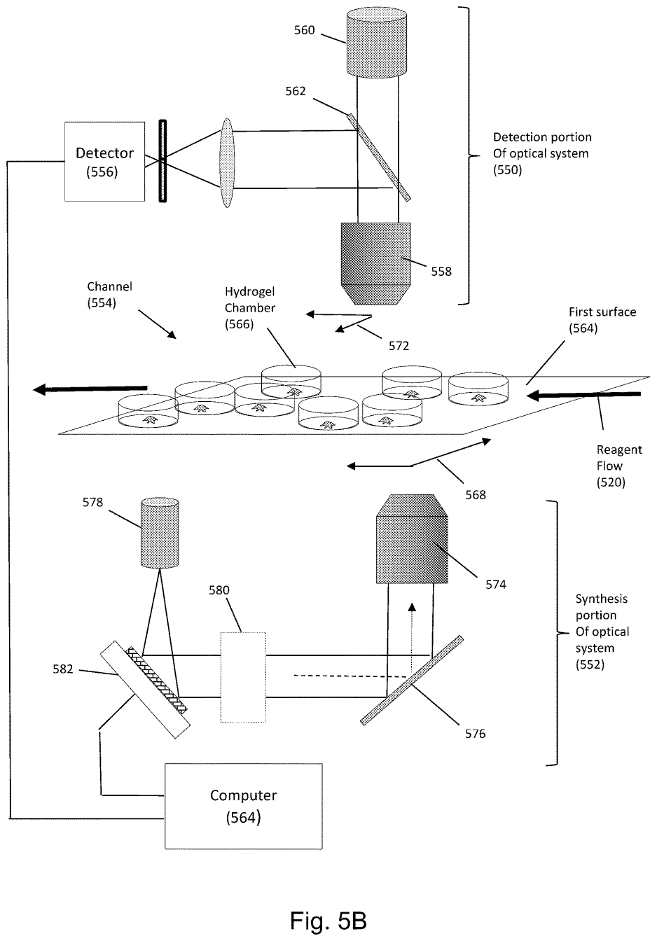

An example system for carrying out the above method is illustrated in FIG. 5A. Flow cell (500) is a component of a fluidic device that provides channels for carrying out a variety of assays and liquid handling components under programmable control for delivering samples and reagents to the channels. In this illustration, four channels (502, 504, 506, and 508) are shown, with blow-up view (512) of segment (510) of channel 2 (504) shown below. In the abstracted view of flow cell (500) of FIG. 5A, inlets, outlets and other features of the channels are not shown. On first surface (514) of channel 2 (504) a plurality of cells, e.g. (518), are each enclosed by a hydrogel chamber, e.g. (516). In some embodiments, the porosity of polymer matrix walls of the hydrogel chambers is selected to be impermeable to the cells, but permeable to assay reagents. Thus, reagents may be introduced to, and removed from, the interiors of the hydrogel chambers by flowing (520) them through the channels, but cell are retained in. Below blow-up (512) of channel segment (510) is shown an example of an optical system (521) for photosynthesizing hydrogel chambers at the locations of cells in the channels and for collecting optical signals including images, in some embodiments. One of ordinary skill in the art would recognize that optical systems with different configurations than those of FIGS. 5A and 5B may be employed for carrying out these functions. In some embodiments, a plurality of DMD-objective subsystems for synthesizing hydrogel structures may be employed to increase the speed of synthesis by synthesizing multiple structures simultaneously.

Returning to FIG. 5A, for photosynthesizing the hydrogel chambers, light source (522) generates light beam (523) of appropriate wavelength light (e.g. UV light) that passes through an appropriate photo-mask or beam-shaping or beam steering (Galvo) system for shaping a beam to synthesize a desired structure or structures in a channel. In some embodiments, a digital micromirror device (DMD) (524) is employed, in other embodiments, a physical photo-mask may be employed. Chamber position, shape and polymer matrix wall thickness is determined at least in part from cell position information determined from images collected by detector (532). Reflected light from DMD (524) is shaped using conventional optics, e.g. collimating optics (528), and is directed through objective lens system (534) into channel 2 segment (510). Objective (534) and flow cell (500) move relative to one another in the xy-directions (536) to photosynthesize chambers at any position in any of the channels. In some embodiments, flow cell (500) moves and optical system (521) is stationary. In some embodiment, objective (534) may also direct light beam (527) from light source (529) to targets, such as cells, on first surface (514) and collect optical signals, such as fluorescent signals, from assays taking place on first surface (514). Alternatively, optical signal collection may be carried out with a separate objective as shown if FIG. 5B. Information collected by detector (532), or its counterpart in the embodiment of FIG. 5B, particularly cellular positions in their respective channels, is employed by computer (538) and/or subsidiary controllers to direct DMD (524) and translation devices controlling the relative positions of objective (534) and flow cell (500) to synthesize hydrogel chambers of the appropriate shape and size at the appropriate locations.

FIG. 5B illustrate an alternative optical system in which the detection portion (550) of the optical system moves (572) independently from the movement (568) of the synthesis portion (552) of the optical system. Detection portion (550) of the optical system comprises detector (556), objective (558), light source (560) and interconnecting optical elements, such as dichroic mirror (562). As with the embodiment of FIG. 5A, detector (556) is operationally associated with computer (564) and the synthesis portion (552) of the optic system to provide synthesis portion (552) with cellular position information. Computer (564) and (538) are also in operationally associated with stages and/or motors controlling the relative positions of the objectives of the optical systems and the position of the flow cell. In this embodiment, synthesis portion (552) of the optical system is located on the other side of first surface (564) from detection portion (550). As with the embodiment of FIG. 5A, it comprises the conventional components objective (574), mirror (576), collimating optics (580), DMD (582) and light source (578).

In some embodiments, cells, e.g. (518) in FIG. 5A, are disposed randomly on first surface (514). In alternative embodiments, first surface (514) may comprise regularly spaced sites or features for capturing cells so that they are disposed substantially only on such sites or features on the first surface. For example, in some embodiments, such sites or features may be a rectilinear or a hexagonal array of spots. In some embodiments, such sites or features may comprise capture elements, such as antibodies specific for cell surface proteins, for capturing or immobilizing cells at the sites or features. In some embodiments, first surface (514) may comprise capture elements designed to immobilize particular cell types. For example, lymphocytes are non-adherent on most surfaces; however, such cells may be allowed to settle on first surface by gravity or may be immobilized on surfaces using capture elements comprising antibodies specific to lymphocyte cell surface proteins, e.g. CD3, CD4, CD8 or the like, e.g. Sekine et al, J. Immunol. Meth., 313 (1-2): 96-109 (2006); Zhu et al, Anal. Chim. Acta, 608:186-196 (2008). For adherent cells, in some embodiments, patterning of fibronectin protein or polylysine on surfaces may be employed to capture such cells at defined locations. In some embodiments, such methods may be used to separate different cellular subsets in different regions within the same channel.

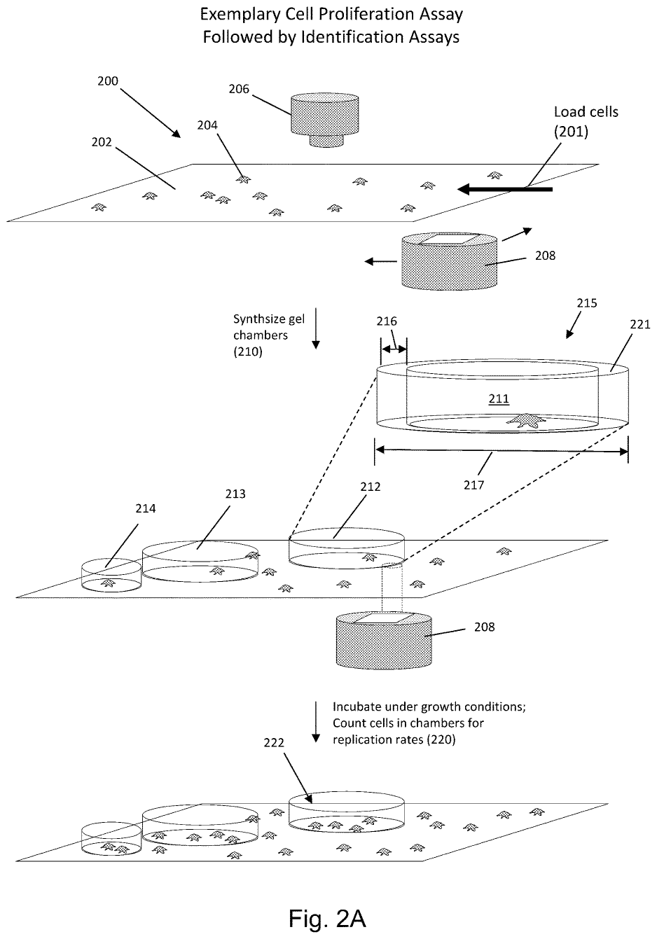

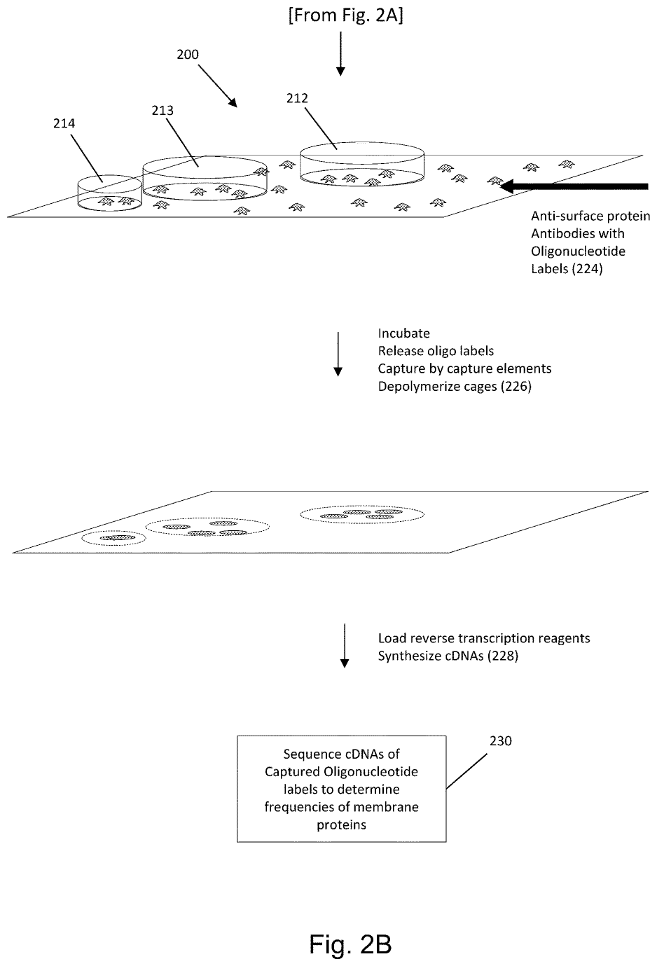

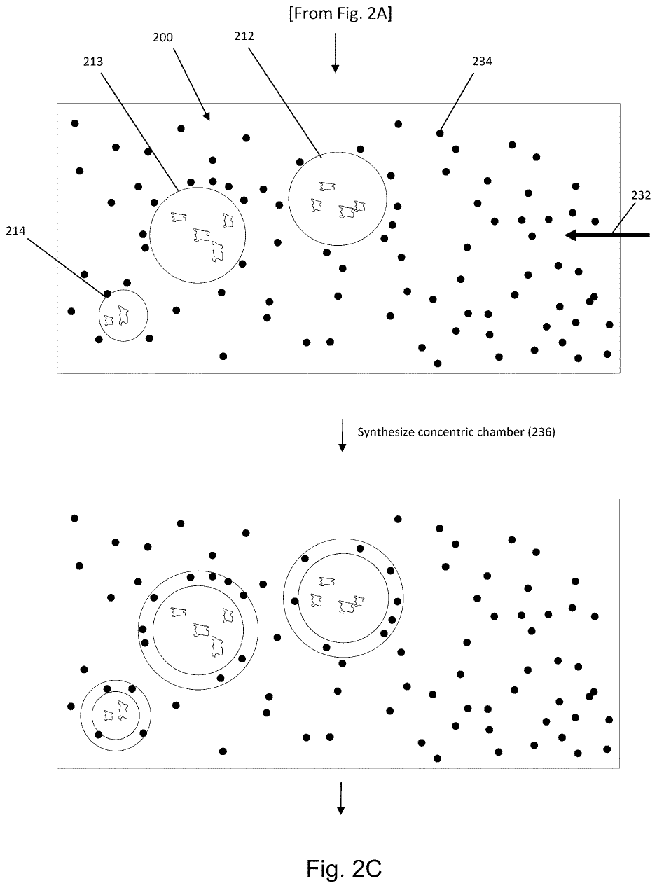

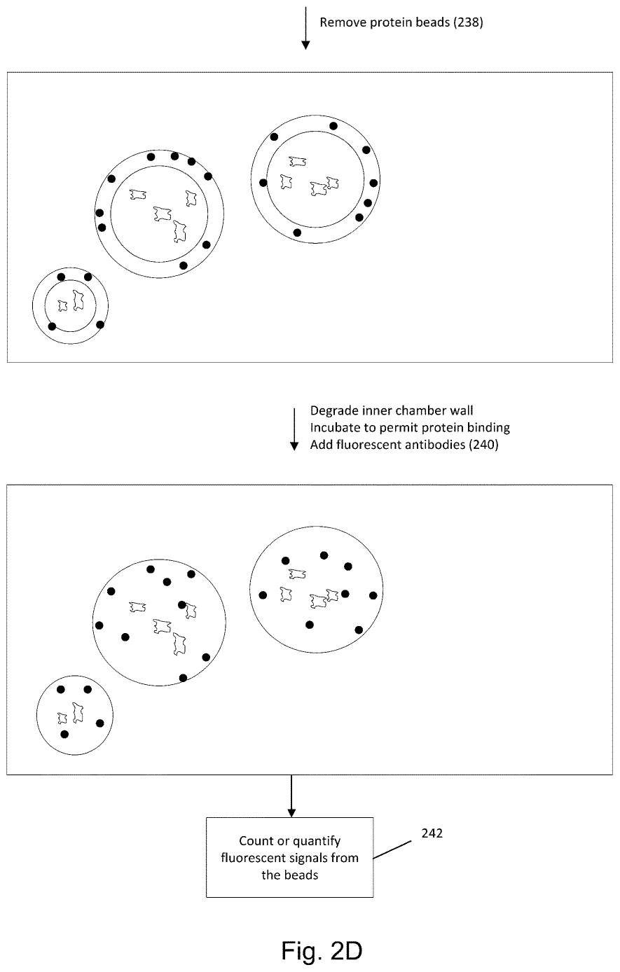

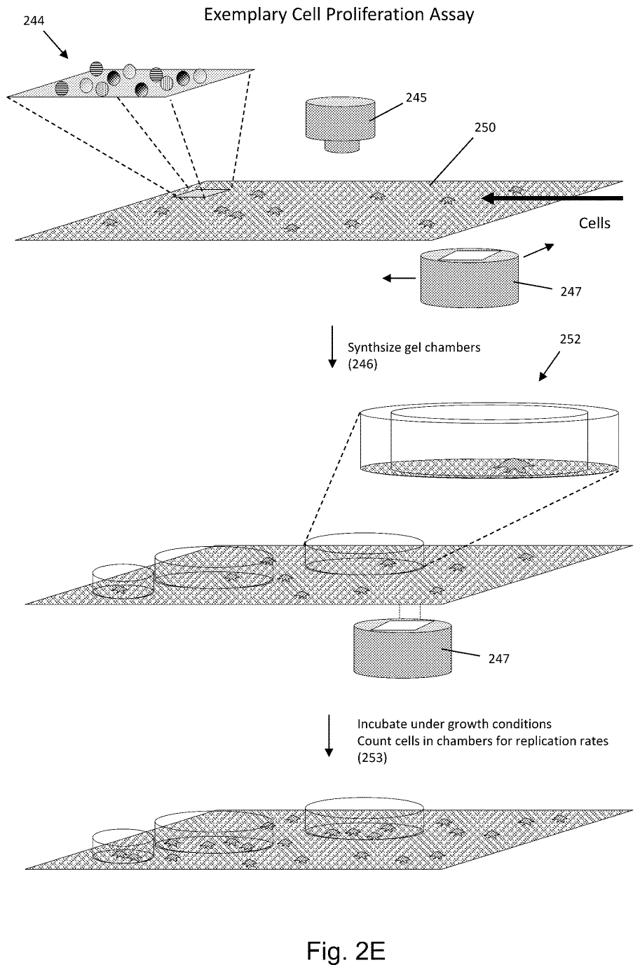

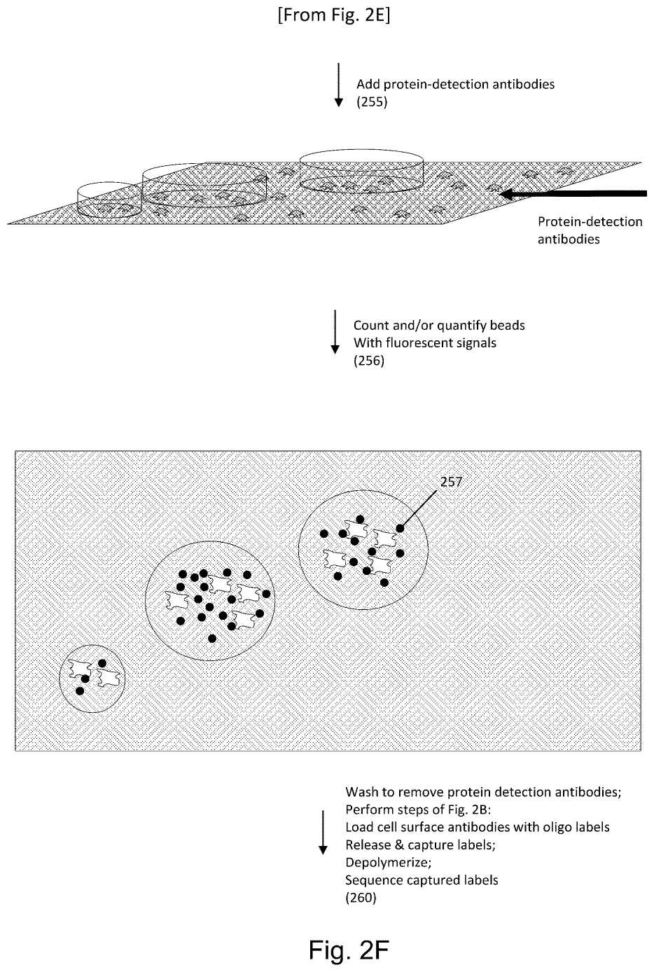

FIGS. 2A-2F illustrate assays for determining proliferation rates of cells followed by their identification based either on cell surface proteins, protein secretion profile, or the like. Cells may also be identified or characterized by their transcriptomes determined as described below or by like techniques. It is understood that the term “proliferation rate” may include a measure of a lack of proliferation. For example, chambers enclosing one or more cells may be exposed to an agent, e.g. a drug candidate, after which such cells may be returned to normal growth conditions. In some cases, the agent may kill or retard the growth of the cells, e.g. in comparison to controls not exposed to the agent. Thus, in the case of the treated cells, a negative “proliferation reate” may be possible because the final numbers of cells counted in the chambers may be less than the original numbers; or a signal monotonically related to cell number may decline in value.

FIG. 2A shows steps of a proliferation assay carried out by a system described herein. Cells and polymer precursors are loaded into channel (200) and disposed on first surface (202), e.g. cell (204). Positions of cells are determined by detector (206) which are used by a control system to generate instructions for spatial energy modulating element (208) to produce light beams to synthesize (210) hydrogel chambers in channel (200) around single cells, as illustrated by hydrogel chambers (212, 213 and 214). Blow-up (215) illustrates that the solid appearing structures (212, 213 and 214) have interiors (211) and walls (221) with predetermine thickness (216). Likewise, hydrogel chambers have a predetermined shape (e.g. circular with diameter (217)) and enclose predetermined areas. In the figures, for convenience, chambers are illustrated as standing in isolation without connection with adjacent chambers and as having a cylindrical or annular-like shapes; however, a spatial energy modulating element may synthesize chambers of different shapes and sizes, as is useful for particular applications. In some embodiments of the proliferation assay, each hydrogel chamber synthesized has the same shape and area, for example, annular-like with an interior area selected from the range of 0.001 to .01 mm², or in the range of 001 to 1.0 mm². In some embodiments, each hydrogel chamber synthesized has the same shape and area for each different type of cell being assayed, for example, cytotoxic T lymphocytes may be confined in chambers having one area whereas helper T lymphocytes may be confined in chambers having another area. After a desired number of hydrogel chambers are synthesized, cells are incubated for a period under growth conditions, after which (for example) cells in each chamber are counted (220) to give a measure of proliferative capacity for each cell. In some embodiments, after synthesizing chambers to enclose selected cells, non-selected cells may be removed by a washing step or by other changes of reagents in the channel. In some embodiments, cells may be stained with a membrane or intracellular dye for determining proliferation by dye dilution so that an independent measure of cell proliferation may be obtained. Example intracellular dyes for dye dilution include, but are not limited to, Hoechst 33342, carboxyfluorescein succinimidyl ester (CFSE), and the like. The desired number of single cells enclosed by chambers depends on statistical confidence desired in the measured values. If a subpopulation of interest is present as only a small fraction of a total population then a larger number chambers is required. In some embodiments in which mammalian cells are assayed the number of hydrogel chambers synthesized around single cells may be greater than 100; or greater than 1000; or greater than 10,000; or the number may be in the range of from 100 to 100,000; or in the range of from 1000 to 100,000. After counts are recorded for each chamber, further assays may be conducted on the clonal populations within the chambers to identify the cell types, for example, by an assessment of cell surface proteins, cell protein secretions, transcriptome, or the like. This approach is particularly useful for assessing populations of immune cells, especially engineered immune cells. In some embodiments, a cell proliferation assay may be carried out by the following steps: (a) providing a fluidic device comprising (i) a channel comprising a first surface, cells disposed on or adjacent to the first surface, and one or more polymer precursors, (ii) a spatial energy modulating element in optical communication with the first surface, and (iii) a detector that identifies positions of cells in the channel based on one or more optical signals therefrom; (b) synthesizing one or more chambers in the channel enclosing each of one or more cells by projecting light into the channel with the spatial energy modulating element such that the projected light causes cross-linking of the one or more polymer precursors to form polymer matrix walls of the chambers, wherein the positions of the synthesized chambers are determined by the positions of cells enclosed thereby identified by the detector; (c) incubating the cells under proliferation conditions; and (d) determining a proliferation rate of the cells. In some embodiments, determining a proliferation rate comprises counting the cells in each chamber.

FIG. 2B illustrates a follow-on assay to identify cells by cell surface markers. It is noted that if cell surface markers are to be measured directly (without first measuring cell proliferation), then prior to introduction to a channel, cells may be incubated with labeled antibodies, wherein the labels may vary widely including, but not limited to, fluorescent labels, oligonucleotide labels, colorimetric labels, enzymatic labels, or the like. Also, it is understood that binding compounds other than antibodies or fragments thereof may be employed, such as, for example, aptamers. After determining proliferation rates for each chamber, channel (200) is loaded with a mixture of different labeled antibodies each specific for a different cell surface protein. Such antibodies may be prepared for any cell surface protein, but surface proteins of special interest are those that characterize immune cells and their binding specificities (e.g. CARs). In some embodiments, antibodies are specific for surface proteins that include, but are not limited to, cluster of differentiation (CD) markers, such as, CD3, CD4, CD5, CD6, CD7, CD8, CD11, CD19, CD20, CD21, CD22, CD23, CD24, CD25, or the like. In some embodiments, the labels of the antibodies are oligonucleotides. In some embodiments, such oligonucleotides may be attached to the antibodies by a scissile bond which, for example, may be cleavable by conventional methods, such as, chemical cleavage by reducing conditions, acid conditions, or base conditions, or photo cleavage by exposure to an appropriate wavelength and intensity of light. In some embodiments, exposing antibody-labeled cells to a lysing reagent is sufficient to release the antibody-oligonucleotide conjugates for capture by capture elements, whether or not a scissile linkage is present. The oligonucleotide labels may comprise a barcode sequence that uniquely identifies the protein for which the antibody is specific. The oligonucleotide labels may also include a sequence complementary to oligonucleotides attached to the first surface as capture elements. In some embodiments, either such complementary sequences, or those of the capture oligonucleotides, may be blocked by hybridization of a mismatch or shorter oligonucleotide (e.g. less stable than the oligonucleotide label-capture oligonucleotide duplex) to prevent spurious capture of the antibody-oligonucleotide conjugates by capture oligonucleotides on the first surface when loaded into a channel. Such blocking oligonucleotides are then removed in a wash step prior to releasing the oligonucleotide labels of the cell surface bound antibodies. Alternatively, oligonucleotide labels may be configured to be ligated to capture oligonucleotides by way of a splice oligonucleotide. The splice oligonucleotide would block spurious hybridization of the oligonucleotide labels prior to release from the antibody-surface protein complexes. After such release and introduction of a ligase, oligonucleotide labels would be captured by the free end of the splice oligonucleotide and the oligonucleotide label would be ligated to the end of the capture oligonucleotide.

In the embodiment of FIGS. 2A-2B, after a period of incubation (226) to permit binding of antibodies to their target proteins, the oligonucleotide labels are released and captured by the complementary sequences of the capture elements. For example, oligonucleotide labels may be attached by a disulfide linker which may be released by a reducing agent, e.g. Hermanson (cited above). In some embodiments, after incubating, unbound antibodies may be removed, for example, by a washing step. In some embodiments, channel (200) may be loaded (228) with a reaction mixture comprising a polymerase, dNTPs and other components, necessary for extending the complementary oligonucleotides of the capture elements with the captured antibody oligonucleotides as templates. In some embodiments, the polymer matrix walls of the chambers may be degraded, or depolymerized (226), after which channel (200) may be loaded (228) with such extension reagents. As explained more fully below, the DNA copies may be sequenced (230) in situ or they may be released from the first surface (perhaps after amplification), eluted, and sequenced externally. In the latter process, the complementary oligonucleotides of the capture elements include spatial barcodes for identifying the chamber from which antibody barcode sequences originate. In either case, the numbers of each kind of antibody oligonucleotides calculated from the sequence data provide profiles (e.g. relative frequencies of each cell surface protein) of the cell surface proteins of the cells enclosed by each of the hydrogel chambers. Moreover, this data can be correlated to the proliferative capacity of such cells.

FIGS. 2C-2D illustrate one embodiment of a protein secretion assay employing commercially available protein-capture beads, e.g. Biolegend (San Diego, CA). One of ordinary skill in the art would recognize that other surfaces beside (or in addition to) bead surfaces may be employed for localized capture of secreted proteins, such as cytokines. The upper and lower panels of FIG. 2C are top views of a section of channel (200) containing chambers (212, 213 and 214). (Thus, FIGS. 2C-2D illustrate an alternative assay of cells after counting (to that of FIG. 2B), or in some embodiments, the assay of 2B could be performed after the assay of FIGS. 2C-2D, so that protein secretion profiles would be determined and afterward surface protein profiles would be determined) After cell counting, channel (200) is loaded (232) with protein-capture beads (e.g. 234) which are beads (usually impermeable to the polymer matrix walls of the chambers) which have covalently attached antibodies specific for predetermined proteins that may be secreted by the cells enclosed by the chambers. Such beads may be prepared for any secreted protein, but proteins of special interest are cytokines and immune active proteins including, but not limited to, interferons, such as interferon-g (IFN-g) and interferon-a (IFN-α), interleukins, such as, interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-13 (IL-13), interleukin-15 (IL-15), and interleukin-21 (IL-21), interleukin-23 (IL-23), colony stimulating factors (CSFs), such as, granulocyte-macrophage colony stimulating factor (GM-CSF), granulocyte colony stimulating factor (G-CSF), tumor necrosis factors (TNFs), such as, TNF-α and TNF-b, and effector molecules, such as, granzyme B. After such beads are loaded, a concentric chamber is synthesized (236) around each of the chambers containing cells, after which beads outside of the concentric chambers are removed (238). In some embodiments, the size of the larger concentric chamber is sufficient to enclose a number of protein-capture beads so that there is a high probability (e.g. greater than 90 percent, or greater than 95 percent, or greater than 99 percent) that at least one bead is present for each different cytokine to be measured. One of ordinary skill would recognize that such size determination depends on the concentration of beads loaded into channel (200). After synthesis of concentric chambers, the polymer matrix walls of the inner chambers are depolymerized (240) so that cells and bead may freely mix so that secreted proteins may be capture by the beads, after which label antibodies (“detection antibodies”) are loaded into the channel to bind to another epitope of the captured proteins (as in a sandwich assay). In some embodiments, porosity of the inner chambers is selected to prevent or inhibit passage of secreted proteins through its polymer matrix walls. In some embodiments, porosities of both the inner chambers and the outer concentric chambers are selected to prevent or inhibit the passage of secreted proteins, and before loading detection antibodies, the polymer matrix walls of the outer chambers are depolymerized to remove the barriers to the detection antibodies binding to captured proteins. Alternatively, in some embodiments, the porosity of the polymer matrix walls of the inner chamber may be selected to allow passage of secreted proteins into the annular area containing the beads. Relative amounts of secreted proteins may be estimated by counting each bead type generating a characteristic fluorescent signal or by integrating each different fluorescent signal across all bead adjacent to the cells of the chamber. This embodiment may be implement by the following steps after cells are disposed on a first surface, either initially or after another assay not destructive of the cells: (a) loading the channel with a second reaction mixture comprising second polymer precursors and protein capture beads comprising protein-capture antibodies that bind proteins secreted by said cells; (b) synthesizing second chambers enclosing each of the one or more chambers by projecting light into the channel with the spatial energy modulating element such that the projected light causes cross-linking of the one or more polymer precursors to form polymer matrix walls of second chambers, wherein the positions of the synthesized second chambers are determined by the positions of the chambers enclosed thereby identified by the detector; and (c) depolymerizing the chambers so that the protein capture beads in each second chambers mix with the cells enclosed by the depolymerized chamber therein. In some embodiments, the (first) polymer precursors may include precursors that form gel linkages degradable by reducing conditions, e.g. structure 2 of Table 2B, whereas the second polymer precursors do not include such precursors. In some embodiments, the above method further includes incubating the cells with the protein capture beads; and detecting protein secreted by the cells by an amount of labeled protein detection antibodies for each protein bound to protein-capture beads adjacent to the cells.

One of ordinary skill recognizes that the function of protein-capture antibodies may be carried out by other affinity reagents that have specific binding capacity, such as aptamers. As used herein, the term “affinity reagent” means a compound capable of specifically binding to a target molecule, such as a protein. Affinity reagents include, but are not limited to, antibodies, antibody fragments, aptamers and like compounds.

An alternative embodiment for measuring protein secretion profiles is illustrated in FIGS. 2E-2F. In this embodiment, a uniform distribution of protein-capture beads is attached to first surface (250) prior to loading cells. As illustrated in blow-up (244), the uniform distribution may include beads with antibodies specific for different proteins. After deposition of cells on surface (250), cells are located by detector (245) and hydrogel chambers are synthesized (246) by spatial energy modulating element (247). Cells are incubated under growth conditions, after which cells in each chamber are counted to determine a proliferation rate (253). As above, secreted proteins are detected by being captured by protein-capture beads and then labeled with detection antibodies (255). In some embodiments, porosity of the polymer matrix walls of the chambers is selected to prevent passage of cells but to allow the free passage of proteins. In other embodiments, such porosity is selected to prevent to passage of both cells and proteins. In the latter embodiment, an added step of depolymerizing the polymer matrix walls is included. Relative amounts of secreted proteins are determined (256) by either counting labeled beads of the different types adjacent to, or in the proximity of (257), the cells of each chamber, or by integrating the different fluorescent signals from the beads adjacent to, or in the proximity of, the cells of each chamber. As mentioned above, in addition to beads carrying protein-capture antibodies, other surfaces capable of carrying secreted protein capture antibodies include, but are not limited to, the first surface, the polymer matrix walls of a chamber, or a second surface. These surfaces capable of attaching secreted protein-capture antibodies are referred to herein as “protein capture surfaces.”

In some embodiments, the above methods for correlating cellular characteristics with proliferation rate may comprise the steps: (a) providing a fluidic device comprising (i) a channel comprising a first surface, cells disposed on or adjacent to the first surface, and one or more polymer precursors, (ii) a spatial energy modulating element in optical communication with the first surface, and (iii) a detector that identifies positions of cells in the channel based on one or more optical signals therefrom; (b) synthesizing one or more chambers in the channel enclosing each of one or more cells by projecting light into the channel with the spatial energy modulating element such that the projected light causes cross-linking of the one or more polymer precursors to form polymer matrix walls of the chambers, wherein the positions of the synthesized chambers are determined by the positions of cells enclosed thereby identified by the detector; (c) incubating the cells under proliferation conditions; and (d) counting cells in each chamber to determine a proliferation rate thereof. In some embodiments, the first surface may comprise one or more capture elements for capturing one or more biological components of said cells, such as, secreted proteins, messenger RNAs, genomic DNA, or the like. In some embodiments, the step of synthesizing may be followed by a washing step to remove unreacted polymer precursors. In some embodiments, after such washing step (or as part of such washing step) cell culture medium may be loaded in the channel. In some embodiments, the above method further comprises: (i) incubating the cells with antibodies specific for surface proteins whose relative expression permits identification of the cells, wherein each of such antibodies has an oligonucleotide label comprising an antibody-specific barcode capable of being captured by the capture element; (ii) optionally loading into the channel a releasing reagent so that oligonucleotide labels of antibodies attached to the one or more cells are released and captured by the capture elements (if the oligonucleotide labels are attached by a scissile linkage); (iii) optionally depolymerizing the polymer matrix walls of the chambers; (iv) loading said channel with reagents to copy said captured oligonucleotide labels to produce complementary DNAs thereof; and (v) sequencing the complementary DNAs to identify the captured oligonucleotide labels. In some embodiments, cDNAs of captured labels may be sequenced at or near their capture locations by the steps of (a) amplifying said complementary DNAs, (b) sequencing the amplified complementary DNAs, and (c) determining relative expression of the surface proteins for said cells of each of said chambers. Oligonucleotide labels may be attached to antibodies (or other binding moieties) with cleavable linkages well-known in the art. Such oligonucleotide labels attached to antibodies by cleavable linkages may be cleaved, or released from the antibody, by cleavage reagents or agents, or (equivalently) releasing reagents or agents, which may be chemical, physical, or electrical in nature. Reagents to copy captured oligonucleotide labels include a DNA polymerase, dNTPs, and associated buffer and salt solution to extend a capture oligonucleotide using an oligonucleotide label as a template. In some embodiments, CDNAs of captured labels may be sequenced on an external sequencing instrument after (optional amplification and) elution from the channel. For the latter sequencing, the capture elements on the first surface comprise spatial barcodes and cDNAs of the captured oligonucleotide labels each comprise a spatial barcode. After sequencing the cDNA-spatial barcode conjugates, the cDNA may be assigned to a chamber based on the spatial barcode.

In other embodiments, cell proliferation may be correlated to protein secretion by the following steps: (a) providing a channel comprising a protein capture surface comprising protein-capture antibodies that bind proteins secreted by the cells; and (b) detecting protein secreted by the cells by an amount of labeled protein detection antibodies for each protein bound to the protein capture surface adjacent to the cells. In some embodiments, protein capture surfaces comprise protein-capture beads.

In some embodiments, cell proliferation rates may be correlated with cellular transcriptomes following the steps described below for converting mRNA from cells to cDNAs and sequencing the resulting cDNAS. Briefly, after the counting step, the following further steps may be implemented: (a) loading the channel with a lysing reagent so that mRNAs of the cells are released and captured by the capture elements; and (b) loading the channel with reverse transcription reagents to copy the captured oligonucleotide labels to produce complementary DNAs thereof; and (c) sequencing the complementary DNAs. It is understood that a sequencing step may comprise additional steps in particular embodiments including, but not limited to, tagmentation, adding adaptors, cleaving the cDNA to form appropriate lengths for sequencing, and the like. In some embodiments, an additional step may be implemented for depolymerizing or degrading the polymer matrix walls of the chambers after mRNA capture. Reverse transcription reagents comprise conventional reagents for reverse transcription; namely, a reverse transcriptase (such as, a Moloney murine leukemia virus (MMLV)), dNTPs, optional RNase inhibitor, buffer. The sequencing step may be carried out at the sites of the captured mRNAs (in situ) or cDNAs may include a spatial barcode and be eluted and sequenced on a separate sequencing instrument (“external” sequencing). For in situ sequencing, further steps may include (i) amplifying the complementary DNAs, e.g. by bridge amplification, or like method, (ii) sequencing the amplified complementary DNAs, e.g. by a sequencing-by-synthesis technique, and (iii) determining relative expression of the mRNAs for the cells of each of the chambers. For external sequencing, further steps may include (i) providing capture elements comprising spatial barcodes, (ii) synthesizing cDNAs comprising spatial barcodes, and (iii) eluting and sequencing the cDNAs and correlating each cDNA with a chamber location by its spatial barcode.

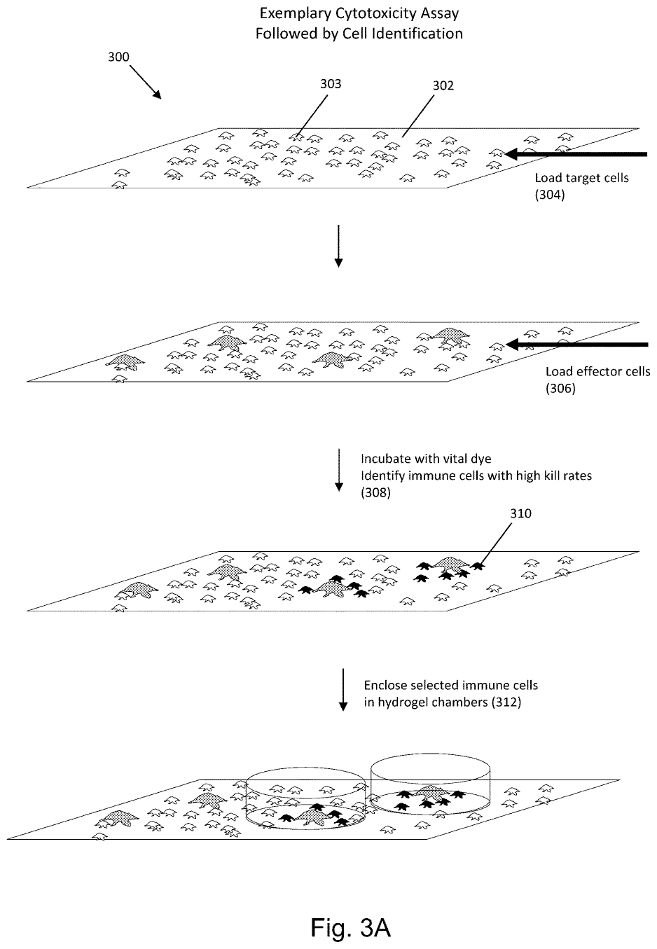

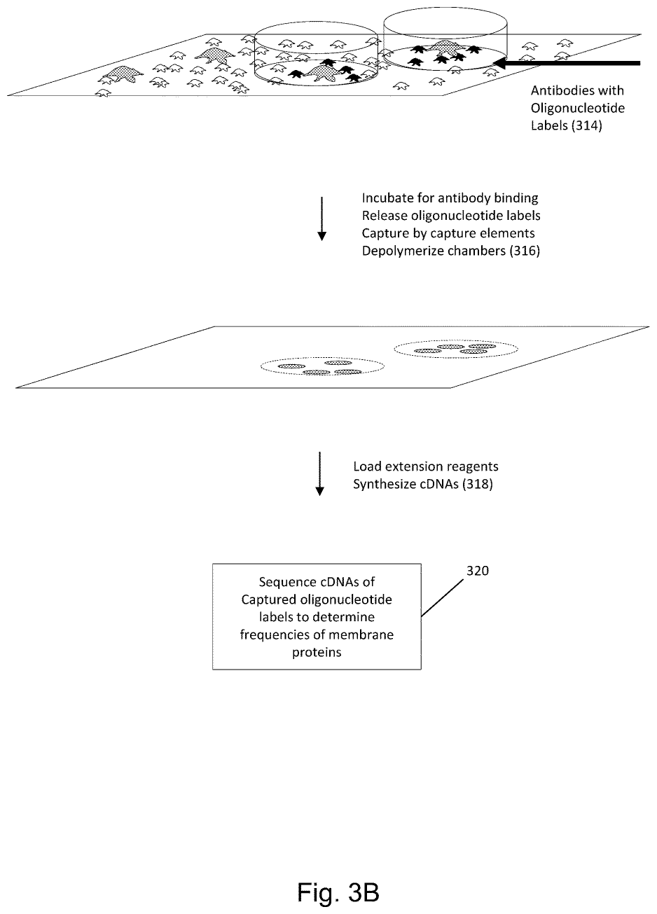

As with cell proliferation, a measure of cell cytotoxicity against a target cells population may be correlated to other cellular characteristics including, but not limited to, cell membrane protein expression, cell protein secretion profile, transcriptome and the like. Target cells may comprise a sample of tumor cells of a patient, or target cells may be from a cell line, e.g. tumor cell line, such as, hepatic tumor cell line, SK-HEP-1, Chava et al, J. Vis. Exp., 2020 Feb. 22: (156): 10.3791/60714. Steps of an example cytotoxicity assay are illustrated in FIGS. 3A-3B. Target cells (303) are loaded (304) into channel (300) so that they are disposed on first surface (302), after which effector cells are loaded (306) and are disposed on first surface (302) along with target cells. “Effector cells” means cells whose cytotoxic capacity is being measured. In some embodiments, effector cells are cells engineered for a therapeutic purpose, such as, treatment of a cancer. Target cells and effector cells are incubated (308) together with a vital dye that generates an optical signal in response to a characteristic of viable cells or dead cells, which permits dead cells to be enumerated. An example vital dye for live cells includes, but is not limited to, Hoechst 33342. An example vital dye for dead cells includes, but is not limited to, 7-AAD, e.g. Schmid et al, Cytometry, 15:12-20 (1994); Bradford et al, poster entitled “Dead cell stains in flow cytometry: a comprehensive analysis,” Molecular Probes (Eugene, OR). After such incubation, a measure of cytotoxicity may be determined by counting dead target cells (310) adjacent to, or in the proximity of, each effector cell. In some embodiments, individual effector cells may be enclosed by chambers immediately after loading to ensure that the only dead target cells counted are those killed by the enclosed single effector cell. In other embodiments, effector cells may be enclosed by chambers at a later time if dead target cells are readily associated with dead target cells, e.g. by proximity. Effector cells may then be identified by (for example) cell surface markers, protein secretions, transcriptome, or the like. For identification by cell surface markers, effector cells may be enclosed by hydrogel chambers (312) (if not already enclosed), and antibodies with oligonucleotide labels may be loaded into the channel (314). In some embodiments, non-enclosed cells may be removed by a washing step or other change of reagents in the channel. After incubation (316) for antibody binding to target surface proteins, oligonucleotide labels are released and captured by capture elements. After such capture, chambers are optionally depolymerized and extension reagent are loaded for copying the captured oligonucleotides by extending capture element oligonucleotides with the captured oligonucleotide as a template (318). The resulting cDNAs are amplified and sequenced (320) as described above for the cell proliferation assays.

Likewise, the cells from different channels enclosed as shown in step (312), which have been measured for cytotoxicity, may be subjected in those different channels to different assays to determine different cellular characteristics from cell surface markers. Such different assays include, but are not limited to, protein secretions, vector copy number, transcriptome, and the like.

In some embodiments, additional steps may be implemented to reduce or eliminate spurious signals generated by proteins or mRNAs of target cells. For example, after incubation for killing and after labeled antibodies are bound to cell surface proteins, effector cells in the chambers may be encapsulated or encased in a gel (or a second chamber may be synthesized) separating it from target cells in the first chamber. After counting, the target cells in the first chamber may then be lysed and removed by washing. After such removal, the gel encasing the effector cells may then be depolymerized to permit antibody labels or mRNAs to be released and captured by capture elements of the first surface. In some embodiments, the gel encasing the effector cells is degradable, e.g. by a reducing agent, and has an average pore size sufficiently small to prevent passage of most mRNA of interest (for example, mRNA of 300 nucleotides or greater) until depolymerization.

In some embodiments, a method of correlating cellular characteristics with cytotoxicity may comprise the steps of: (a) providing a fluidic device comprising (i) a channel comprising a first surface and a population of target cells disposed thereon, (ii) a spatial energy modulating element in optical communication with the first surface, and (iii) a detector that identifies positions of cells in the channel based on one or more optical signals therefrom; (b) loading each channel with effector cells and one or more polymer precursors so that the effector cells are disposed on or adjacent to the first surfaces; (d) synthesizing one or more chambers in the channels each enclosing an effector cell by projecting light into the channel with the spatial energy modulating element such that the projected light causes cross-linking of the one or more polymer precursors to form polymer matrix walls of the chambers, wherein the positions of the synthesized chambers are determined by the positions of effector cells enclosed thereby identified by the detector; (e) incubating the target cells and the effector cells with a vital stain that identifies dead cells; and (f) counting dead cells in each chamber to determine a measure of cytotoxicity of the effector cell enclosed thereby. In some embodiments, the vital stain generates an optical signal in dead cells but not living cells. In some embodiments, the step of synthesizing may be followed by a washing step to remove unreacted polymer precursors. In some embodiments, after such washing step (or as part of such washing step) cell culture medium may be loaded in the channel.

In some embodiments, after an effector cell's cytotoxic capacity is measured, e.g. by counting adjacent, or in-chamber, dead cells, the effector cell may be identified by other assays that measure characteristics such as surface proteins, protein secretion, and the like. Assays for the identification of effector cells by their cell surface proteins may include the following steps: (i) incubating the cells with antibodies specific for surface proteins whose relative expression permits identification of the cells, wherein each of such antibodies has an oligonucleotide label comprising an antibody-specific barcode capable of being captured by the capture element; (ii) loading into the channel a releasing reagent so that oligonucleotide labels of antibodies attached to the one or more cells are released and captured by the capture elements; (iii) optionally depolymerizing the polymer matrix walls of the chambers; (iv) loading the channel with reagents to copy the captured oligonucleotide labels to produce complementary DNAs thereof; and (v) sequencing the complementary DNAs to identify the captured oligonucleotide labels, and thereby determine a cell surface protein profile of each effector cell. As noted above, in some embodiments, before the loading of a releasing reagent, the following steps may be performed: (a) encasing the effector cell of each chamber in a degradable encasing gel that prevents the escape of bound oligonucleotide labeled antibodies or mRNAs, (b) lysing target cells by loading a lysing reagent into the channel, and (c) a washing step to remove target cell bound oligonucleotide-labeled antibodies and mRNAs.

In some embodiments, such cDNAs may be sequenced in situ with the following steps: (a) amplifying the complementary DNAs, (b) sequencing the amplified complementary DNAs, and (c) determining relative expression of the surface proteins for the cells of each of the chambers. In alternative embodiments, such cDNAs may be sequenced externally as follows: providing capture elements with spatial barcodes so that the cDNAs synthesized from the captured oligonucleotide labels include spatial barcodes, amplifying the cDNAs, releasing (or de-hybridizing) and eluting the amplified cDNA (or their complements), sequencing the cDNAs, wherein the spatial barcode permit the identification of the chambers from which the cDNAs originated.

Cytotoxicity of cells may be correlated to protein secretion profiles using the additional steps as described for cell proliferation. In one embodiment, such steps may comprise: (i) loading the channel with a second reaction mixture comprising second polymer precursors and protein capture beads comprising protein-capture antibodies that bind proteins secreted by the cells; (ii) synthesizing second chambers enclosing each of the one or more chambers by projecting light into said channel with said spatial energy modulating element such that the projected light causes cross-linking of the one or more polymer precursors to form polymer matrix walls of second chambers, wherein the positions of the synthesized second chambers are determined by the positions of the (initial) chambers enclosed thereby identified by the detector; and (iii) depolymerizing the chambers so that the protein capture beads in each second chambers mix with the cells enclosed by the depolymerized chamber therein. The above method may further include: incubating the cells with the protein capture beads; and detecting protein secreted by the cells by an amount of labeled protein detection antibodies for each protein bound to protein-capture beads adjacent to said cells.

In alternative embodiments, the channel further comprises disposed thereon protein capture beads comprising protein-capture antibodies that bind proteins secreted by the cells; and the method further comprises detecting protein secreted by the cells by an amount of labeled protein detection antibodies for each protein bound to protein-capture beads adjacent to said cells.

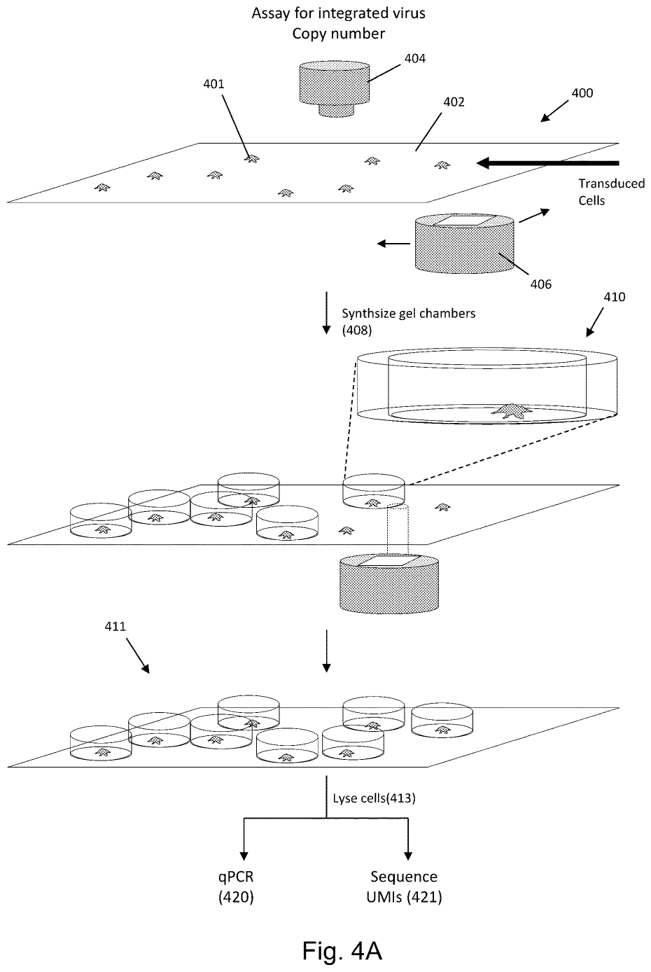

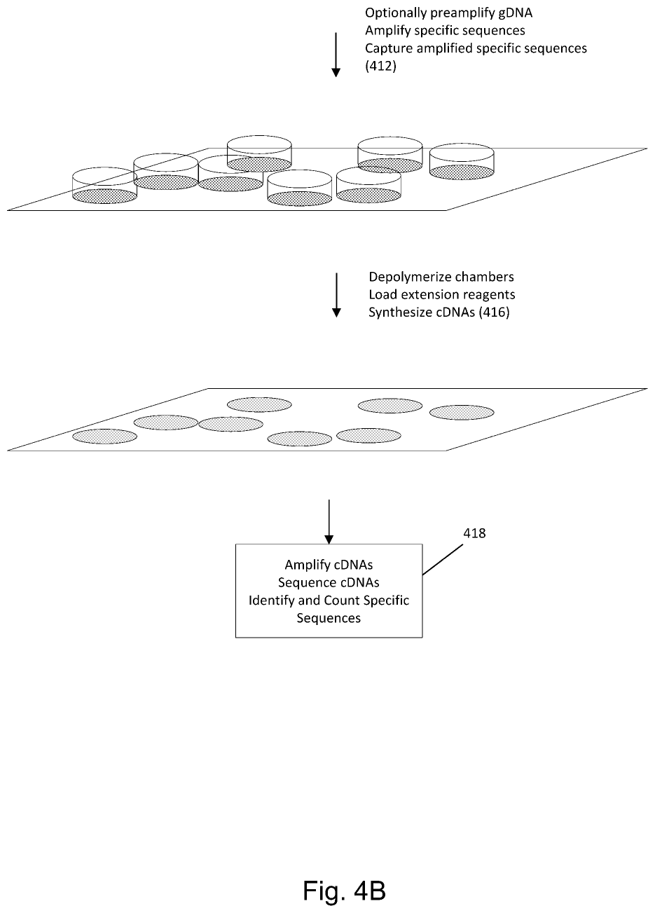

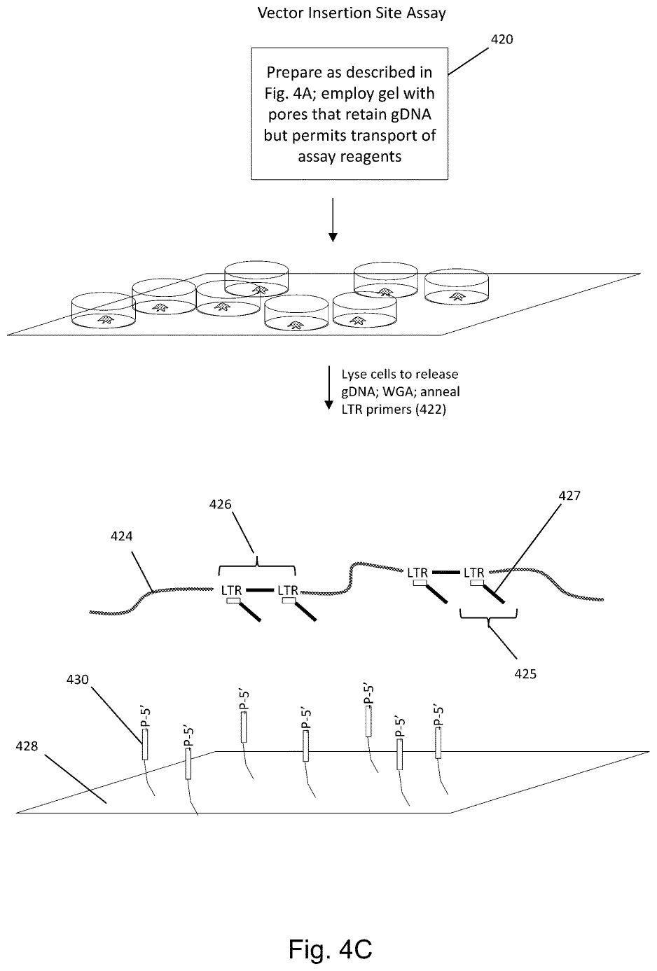

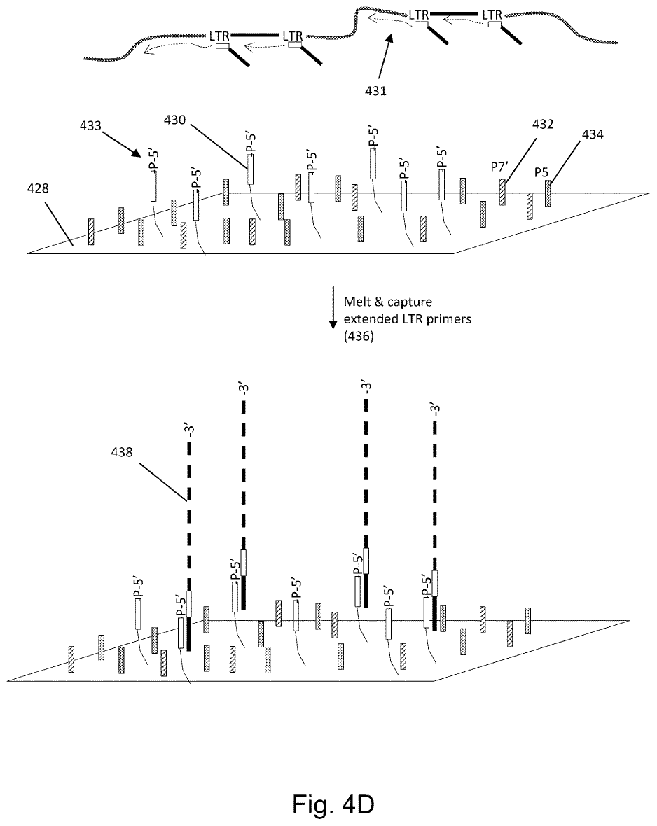

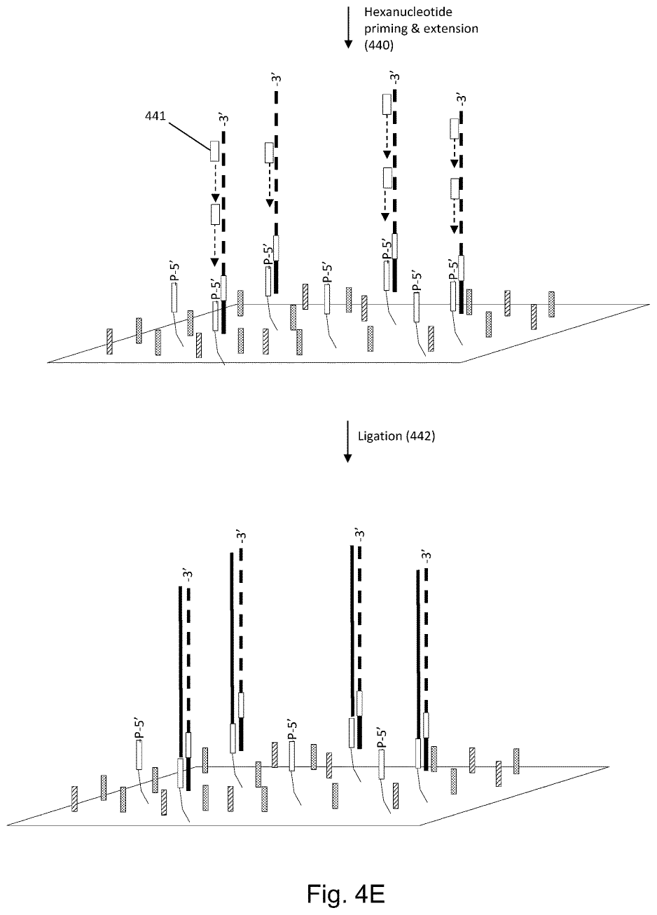

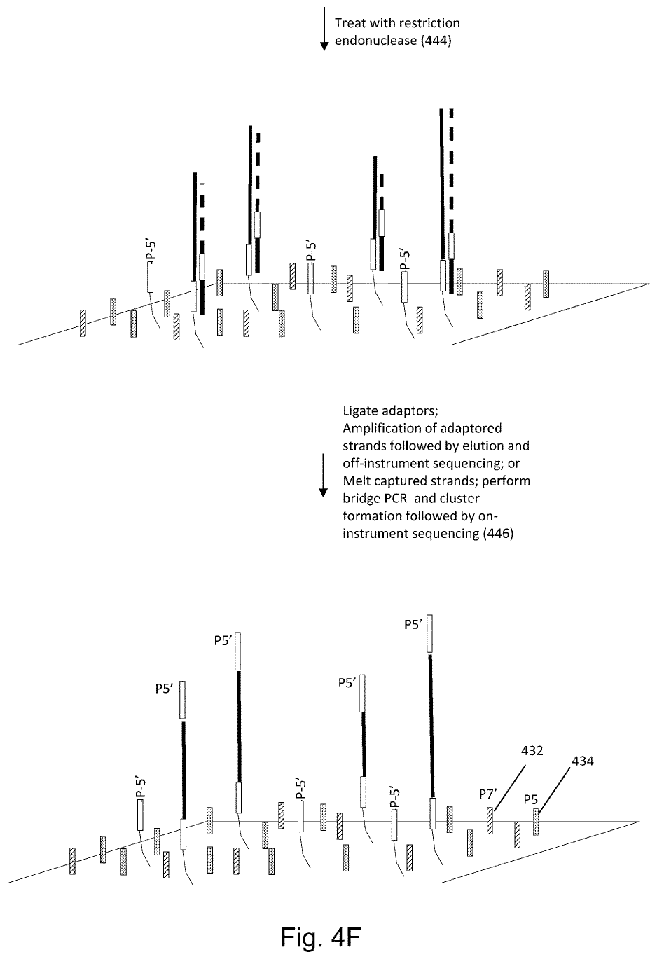

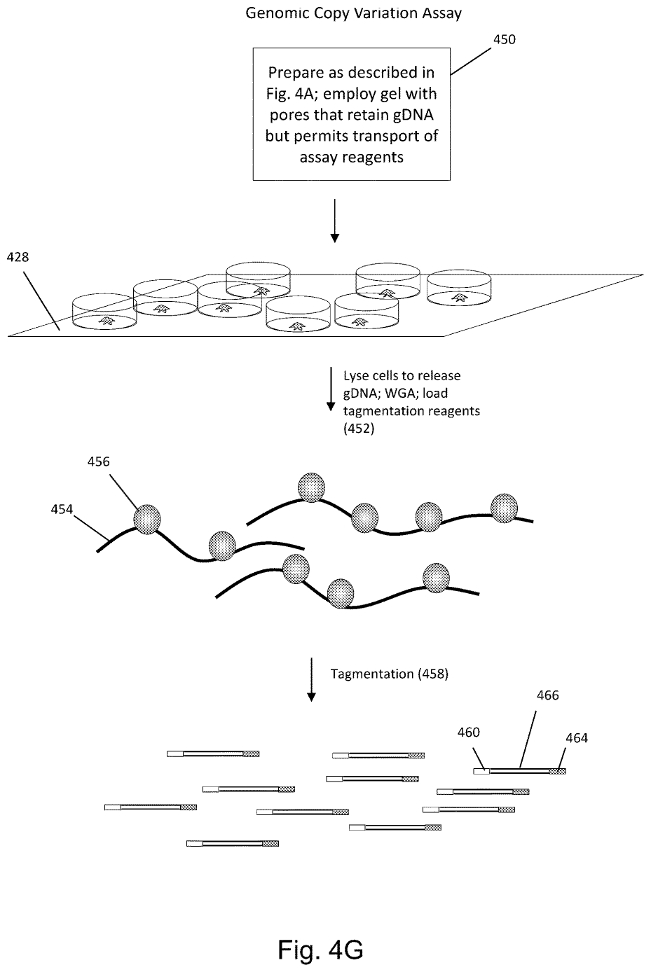

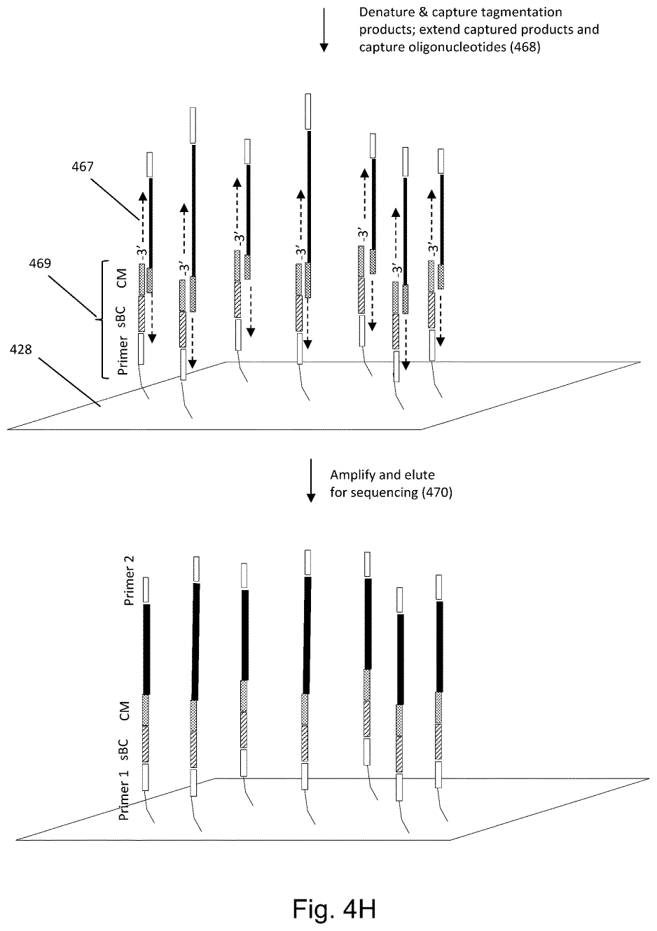

Vector copy number in cells engineered for therapy is important because of the increased risk of oncogenesis if copy number is too high, such as, higher than about five copies per cell, e.g. Chen et al, Cytotherapy, 22 (5), supplement, S142 (2020); Paugh et al, Scientific Reports, 11:389 (2021). In accordance with the systems and methods described herein, vector copy number may be measured by single cell quantitative PCR in chambers or by counting unique molecular identifiers (UMIs) amplified and sequenced from single cells in chambers or like measure. For either assay cells are prepared as illustrated in FIG. 4A. Namely, engineered or transduced cells are loaded into channel (400) so that cells (e.g. 401) are disposed onto first surface (402), after which detector (404) records the locations of cells on first surface (402) and spatial energy modulating element (406) synthesizes (408) chambers (411) separately enclosing single cells. Polymer precursors and conditions are selected so that the porosity of the polymer matrix walls of the chambers permit the transit of proteins, lysis reagents, mRNAs, and the like, but retain genomic DNA, for example, using formulations as described in Spencer et al, ISME Journal, 10: 427436 (2016); and Tamminen et al, Frontiers Microbiology: Methods, 6:195 (2015). Cells may be lysed (413) as described in Tamminen et al (protease K followed by lysozyme) or as described by Zhang et al, Proc. Natl. Acad. Sci., 89:5847-5851 (1992) (alkaline lysis: 200 mM KOH/50 mM dithiothreitol followed by neutralization with 900 mM Tris-HCL, pH 8.3/300 mM KCl/200 mM HCl); or like methods. After such treatment, genomic DNA retained in the chambers may be further analyzed by qPCR (420), such as described by Vaninsberghe et al, PlosOne, 13 (1): e0191601 (2018), or by sequencing (421) as outlined in FIG. 4B, if the vectors comprise UMIs, e.g. Porter et al, Genome Biology, 15: R75 (2014).

For qPCR (420), in some embodiments, channel (400) is loaded with a PCR reaction mixture including polymerase, primers, taqman probe, buffers and salts. In some embodiments, to run the PCR, the flow cell is moved to a temperature cycling station. As the PCRs progress in the various chambers, detector (404) records the accumulation of signal from the taqman probes at each chamber, which can be converted into copy numbers. In some embodiments, the channel containing chambers in which qPCR reactions are occurring may be loaded with a blocking agent to prevent diffusion of labeled taqman fragments from the chambers. Such blocking agent may include polymer precursors which may be polymerized in the spaces surrounding the chambers.

For sequencing (412), optionally, the retained genomic DNA may be pre-amplified by a whole genome amplification technique, e.g. as described by Zhang et al (cited above), after which desired sequences, e.g. those containing the virus UMIs, are specifically amplified (e.g. by PCR or a linear amplification technique) and captured by capture elements (412). In some embodiments, the UMI along with a “handle” segment (an oligonucleotide segment complementary to a capture oligonucleotide) is amplified to facilitate the capture by a capture element of the first surface. As describe above, chambers may be depolymerized, extension reagents loaded, and cDNAs synthesized (416) which contains a copies of the UMIs. After amplification, cDNAs are sequenced either in situ or externally and the number of UMIs with different sequences are determined to give a virus copy number for the cells of each chamber.

The above methods may be implemented by first enclosing the engineered cells in hydrogel chambers, namely, by the steps of (a) providing a fluidic device comprising (i) one or more channels each comprising a first surface, (ii) a spatial energy modulating element in optical communication with each first surface, and (iii) a detector that identifies positions of cells in each channel based on one or more optical signals therefrom; (b) loading each channel with cells and one or more polymer precursors so that the cells are disposed on or adjacent to the first surfaces; (c) synthesizing one or more chambers in each channel, each chamber enclosing a cell by projecting light into each channel with the spatial energy modulating element such that the projected light causes cross-linking of the one or more polymer precursors to form polymer matrix walls of the chambers, wherein the positions of the synthesized chambers are determined in each channel by the positions of the cells enclosed thereby identified by the detector; (d) loading one or more channels with assay reagents (such as, for example, lysing reagents, followed by amplification reagents, followed by cDNA synthesis reagents, and so on); and (e) incubating the cells in each channel under assay conditions, which include PCR conditions, rolling circle amplication conditions and/or sequencing conditions, to generate signals from chambers of each channel indicative of a sequence copy number, such as, a vector copy number (VCN). For enumerating copy numbers of specific (or predetermined) nucleotide sequences, such as UMIs, in some embodiments, the following steps may be implemented: (i) lysing the cells to release genomic DNA; (ii) amplifying the one or more nucleotide sequences; (iii) capturing the amplified one or more nucleotide sequences by the capture elements; (iv) loading the channels with reagents to copy captured one or more nucleotide sequences to produce complementary DNAs thereof; and (v) sequencing the complementary DNAs to identify the copy numbers of the one or more nucleotide sequences. Embodiments of special interest comprise predetermined nucleotide sequences that are barcodes, especially barcodes that are or comprise UMIs. The amplified nucleotide sequences typically comprise one or two primer binding sites, a barcode and a sequence complementary to the capture oligonucleotides of the capture elements. The amplified nucleotides may include additional segments for increasing the molecular weight of the amplified products to prevent or inhibit passage through the polymer matrix walls of the hydrogel chamber. From the sequencing data the number of different UMIs may be identified which gives the viral copy number for the cell.

For enumerating copy numbers by quantitative PCR or other amplification methods, in some embodiments, the following steps may be implemented: (i) lysing the cells to release genomic DNA; (ii) loading said channels with amplification reagents that generate a signal proportional to a copy number of the one or more nucleotide sequences; (iii) amplifying the one or more nucleotide sequences to generated signals monotonically related to the copy numbers of the one or more nucleotide sequences. In some embodiments, the amplification reagents may be quantitative PCR reagents, bridge PCR reagents, or rolling circle amplification reagents. In some embodiments, quantitative PCR reagents comprise an amplification buffer, a polymerase with 5′□13′ exonuclease activity, primers, dNTPs and a taqman probe

In further embodiments, copy numbers may be determined by copying the selected genomic sequences, capturing them with capture elements, synthesizing cDNAs, then either forming clusters of the cDNAs by bridge PCR or DNA nanoballs by rolling circle amplification, after which the clusters or DNA nanoballs may be counted for a measure of copy number. Whenever quantitative PCR is employed a signal related to copy number may be an optical signal or a cycle number. Whenever bridge PCR is employed the first surface comprises bridge PCR primers and a signal related to copy number may be the number of clusters formed. Whenever rolling circle amplification is employed a signal related to copy number may be a number of DNA nanoballs formed. Clusters and DNA nanoballs may be detected by a wide variety of techniques including, but not limited to, use of fluorescently labeled dNTPs, double stranded DNA dyes, and the like.

The site or sites of integration of viral vectors in the genomes of therapeutic cells is of vital interest because of possible disabling or altering of the expression of one or more important genes, which is sometimes referred to as genotoxicity or insertional mutagenesis, e.g. Biasco et al, Molecular Therapy: Methods & Clinical Development, 8:21-30 (2018); Cornetta et al, Molecular Therapy: Methods & Clinical Development, 28:28-39 (2023); Desfarges et al, Viruses, 2:111-130 (2010); and the like. Insertion sites may be detected in populations of cells, as exemplified in FIGS. 4C-4F. The general concept of the measurement approaches is to anneal primers to known sequences of vectors integrated into the genome, extend the primers into the genomic DNA of the host, then identify the host gDNA and the site of integration. Individual cells are enclosed in hydrogel chambers as described in FIG. 4A, wherein hydrogel porosity is selected (420) so that the walls of the chamber prevent the passage of genomic DNA, but permit the passage of reagents, such as deoxynucleoside triphosphates (dNTPs), DNA polymerase, primers, and the like, for example, as disclosed by Spencer et al, ISME J., 10:427-436 (2010). Cells are lysed (422) to release genomic DNA (gDNA) (424), after which the released gDNA is amplified, denatured and combined with vector-specific primers (e.g., 425). Such lysing may be carried out with a variety of methods known in the art by loading lysing reagent into channels, e.g. Spencer (cited above); Cui et al, Proc. Natl. Acad. Sci., 86:9389-9393 (1989); Deleye et al, Scientific Reports, 5: 11711 (2015); and the like. After lysing, whole genome amplification (WGA) may be performed using techniques known in the art, e.g. primer extension preamplification PCR (PEP-PCR), degenerate oligonucleotide primer PCR (DOP-PCR), multiple displacement amplification (MDA), multiple annealing and looping based amplification cycles (MALBAC), or the like, disclosed in the following references, Volozonoka et al, Int. J. Mol. Sci., 23:4819 (2022); Hou et al, GigaScience, 4:37 (2015); Yu et al, Anal. Chem., 86:9386-9390 (2014); or the like. FIG. 4C shows integrated vectors (426) as linear segments of DNA flanked by long terminal repeat (LTR) sequences. In some embodiments, other vector regions may be used as primer binding sites. LTR primers (425) have 5′ oligonucleotide tails (427) that have sequences complementary to capture oligonucleotides (430) on first surface (428) within the chamber. LTR primers are extended by a DNA polymerase in the presence of dNTPs (e.g. 431), after which the chambers are washed at a stringency (e.g. salt, heat, etc.) high enough to destabilize and remove non-extended LTR primers but not so high as to remove extended LTR primers (429). Extended LTR primers are then melted from the gDNA and are captured (436) by capture oligonucleotides (430) by hybridizing to the 5′ tails (427) of the extended LTR primers. The capture oligonucleotides (430) are attached to surface (428) by their 3′ ends and their 5′ ends have attached 5′-phosphate groups. Capture oligonucleotides may have additional features depending of embodiment. For example, if sequences of the extension regions of the extended LTR primers are to be determined in situ, capture oligonucleotides may include primers for bridge amplification. On the other hand, if sequences of the extension regions are to be determined off-instrument, capture oligonucleotides may include spatial barcodes for identifying sequences associated with the same cell. In FIG. 4D, surface (428) is illustrated with so-called P7′ (432) and P5 (434) sequences for bridge PCR; thus, capture oligonucleotides (430) would include P7 sequences for initiating bridge PCR after LTR primer extensions are copied and removed. The actual sequences of the P5 and P7′ regions is a matter of design choice for one of ordinary skill in the art.

After capture of extended LTR primers (436), random-sequence hexanucleotide primers (441) are annealed to extension regions (438) and are themselves extended (440), after which the synthesized strand is ligated to the 5′ end of the capture oligonucleotides, e.g. using protocols described by Schmidt et al, Nature Methods, 4 (12): 1051-1057 (2007); Kalle et al, U.S. Pat. No. 6,514,706; which are incorporated herein by reference. Alternatively, the 3′ ends of extension regions (438) may be extended by a terminal deoxynucleotidyltransferase (TdT) to generate a homopolymer tail to which a complementary primer may be annealed and extended to the 5′ end of capture oligonucleotide (430). The copied sequence is then ligated to capture oligonucleotide (430), after which the resulting construct is treated with a restriction endonuclease to produce a known sequence end distal to surface (428). The restriction endonuclease is selected to maximize the probability of retaining enough extension region for uniquely identifying its location in the engineered cell's genome (e.g. about 18 nucleotide for human). In some embodiments, a restriction endonuclease is selected which has a 4-nucleotide recognition site and leaves a 4-nucleotide overhang after cleavage. After such cleavage, double stranded adaptors are ligated to the ends of the double stranded fragments attached to surface (428). As noted in FIG. 4F (446), the sequences of the adaptored strands may be determined in situ by conducting a surface amplification followed by sequencing reactions, or they may be determined off surface (428) by amplifying by PCR and eluting the amplicon. In the former case, capture oligonucleotides (430) include P7 sequences at their ends proximal to surface (428). In the latter case, capture oligonucleotides (430) include spatial barcodes at their ends proximal to surface (428).

In some embodiments, the above method for vector integration site determination for a population of cells may be implemented by (a) synthesizing one or more hydrogel chambers enclosing each of one or more cells disposed on a surface of a channel;
(b) lysing the cells so that genomic DNA of each cell is released into its hydrogel chamber; (c) amplifying the genomic DNA of each cell; (d) annealing a vector-specific-primer to the amplified genomic DNA, (e) extending the vector-specific primer so that an extension product is formed that includes a copy of a segment of said genomic DNA, and (f) identifying from the segment a site of each vector integrated into said genomic DNA of each said cell. In some embodiments, a vector-specific primer comprises a primer complementary to a vector sequence but not to a genomic sequence of the cell. In some embodiments, whenever a vector is a retrovirus, a vector-specific primer may be complementary to a sequence of a long terminal repeat (LTR) element. Such vector-specific primers are sometimes referred to herein as “LTR primers.” One of ordinary skill would recognize that the degree, or length, of the extension of a vector-specific primer into an adjacent region of genomic DNA must be of a magnitude sufficient for identifying uniquely the position of the integrated vector in a cellular genome. For human genomes, in some embodiments, the length of such extension comprises at least 18 nucleotides of cellular genomic DNA.

An important concern with stem cell therapy is the genetic stability of the stem cells, as they can display aneuploidy and genomic copy number variability, common features of tumorigenic cells e.g. Goldring et al, Cell Stem Cell, 8:618-628 (2011). Single cell genomic copy number variation may be detected in populations of cells in accordance with the invention, as exemplified in FIGS. 4G-4H. In some embodiments, the method described herein comprises steps described in Example 3 of Khurana et al, U.S. patent publication US2022/0219170, which is incorporated herein by reference. As above, individual cells are enclosed in hydrogel chambers as described in FIG. 4A (450), wherein hydrogel porosity is selected so that the walls of the chamber prevent the passage of genomic DNA, but permit the passage of reagents, such as deoxynucleoside triphosphates (dNTPs), DNA polymerase, primers, WGA reagents, tagmentation reagents, and the like, for example, gel porosity as disclosed by Spencer et al, ISME J., 10:427-436 (2010). As shown in FIG. 4G, after cells are enclosed in hydrogel chambers, they are lysed to release genomic DNA, after which WGA reagents are loaded for increasing the amounts of gDNA in the chambers, so that after fragmentation, the fragments sequenced will provide enough coverage for the copy number variation (CNV) to be determined at an acceptable resolution. In some embodiments, a sequencing depth of 0.3X results in 3 megabase (Mb) resolution for CNV determination, e.g. Deleye et al, Scientific Reports, 7:3422 (2017). For 0.3X coverage in human cells about 1 billion nucleotides of sequence reads are required. One of ordinary skill would recognize that the degree of amplification from WGA, surface area enclosed by chambers, and capture oligonucleotide density may be adjusted to achieve a desired CNV resolution. In some embodiments, for CNV measurement the surface area enclosed by chambers is at least 103 mm², or at least 104 mm², or at least 105 mm², or at least 10⁶ mm², and the density of capture oligonucleotides is at least 1×10¹² capture oligonucleotides/cm2. In some embodiments, WGA increases the amount of genomic DNA in each chamber by a factor of at least 100. From such genomic sequence data one can estimate genomic copy number variation by known methods, e.g. Mallory et al, Genome Biology, 21:208 (2020); Wang et al, Briefings in Bioinformatics, 19 (5); 731-736 (2018); or the like. In some embodiments, the resolution of CNV measurement is 3 megabases (Mb) or higher, or 2 Mb or higher, or 1 Mb or higher (where a higher resolution corresponds to a smaller unit of genomic length, e.g. smaller Mb value).

Returning to FIG. 4G, the amplified gDNA (454) is treated with tagmentation reagents, e.g. Tn5 transposase (456), which generates gDNA fragments (466) having upstream (460) and downstream (464) adaptors of predetermined sequences that may include amplification primer binding sites, sequencing primer binding sites, and the like.

The adaptored tagmentation fragments at then denatured so that strands may be captured by capture oligonucleotides (469) and extended (467). Capture oligonucleotide (469) comprises capture moiety (“CM”), spatial barcode (“sBC”) and amplification primer (“Primer 1”). The capture oligonucleotides (469) depicted are on a subregion of surface (428) enclosed by a chamber. Of course, the entire surface (428) may have such capture oligonucleotides attached, whether or not enclosed by a chamber. A second amplification primer (Primer 2″) is provided by the captured tagmentation fragment.

These sequences may be amplified by a conventional PCR and the amplicon eluted (470) from the channel for sequencing. One of ordinary skill would recognize that such elution and/or amplification may be preceded by degrading the hydrogel of the chamber.

In some embodiments, the above method for genomic copy number determination for a population of cells may be implemented by (a) synthesizing one or more hydrogel chambers enclosing each of one or more cells disposed on a surface of a channel; (b) lysing the cells so that genomic DNA of each cell is released into its hydrogel chamber; (c) amplifying the genomic DNA of each cell; (d) sequencing fragments of said amplified genomic DNA; and (e) determining genomic copy number variation for each said cell from the sequences of the genomic DNA fragments. In some embodiments, the step of sequencing comprises fragmenting genomic DNA and attaching adaptors to genomic DNA fragments by tagmentation. In some embodiments, such adaptors comprise strands that are complementary to oligonucleotides of capture elements. In some embodiments, the step of sequencing comprises acquiring a sequence coverage of 0.25X of the cellular genomic DNA or greater. In some embodiments, the genomic copy number variation is determined to a resolution of 3 megabases or higher.

Oligonucleotide labels, barcodes, genomic fragments, messenger RNAs and similar polynucleotide targets may be sequenced by methods and systems described herein. In some embodiments, capture elements for this purpose include oligonucleotides attached to a surface in the channel, wherein such oligonucleotides comprise a sequence segment that is complementary to that of the nucleic acids to be captured, which may be a polyA segment of mRNAs or an arbitrary sequence “handle” sequence region adjacent to a barcode or oligonucleotide label. When sequencing operations are to be performed channels are provided with such capture elements. Such capture oligonucleotides may be attached to a first surface by many chemistries known in the art, e.g. Integrated DNA Technologies brochure entitled

“Strategies for attaching oligonucleotides to solid supports,” (2014). The sequencing step may be performed on the surface of a channel (“in situ” sequencing) or templates may be optionally amplified, released and eluted from the channel and sequenced on an external sequencing instrument (“external” sequencing). In the latter approach, capture elements may include a spatial barcode that provides channel position information, and permits externally determined sequences to be associated with individual chambers. In some embodiments, spatial barcodes are present in sufficiently high density such that each chamber covers an area of the first surface that is uniquely associated with one or more spatial barcodes, and usually a single spatial barcode. In some embodiments, the preparation of polynucleotides for a sequencing operation takes place after the target templates (e.g. oligonucleotide label, mRNAs, genomic fragments) are released and captured by complementary sequences in the capture elements. A releasing step depends on the nature of the target templates. For example, oligonucleotide labels attached to antibodies by a disulfide linkage may be released by a reducing agent (which may be the same as a lysing reagent). mRNAs may be release by treating cells with conventional lysing agents. Releasing genomic fragments may require lysing and pre-amplification steps. Lysing conditions may vary widely and may be based on the action of heat, detergent, protease, alkaline, or combinations of such factors. The following references provide guidance for selection of lysing reagents, or lysing buffers, for single-cell lysing conditions for mRNA and/or genomic DNA: Thronhill et al, Prenatal Diagnosis, 21:490-497 (2001); Kim et al, Fertility and Sterility, 92:814-818 (2009); Spencer et al, ISME Journal, 10:427-436 (2016); Tamminen et al, Frontiers Microbiol. Methods, 6: article 195 (2015); and the like. Example lysis conditions include the following: 1) cells in H₂O at 96 ° C for 15 min, followed by 15 min at 10 °C.; 2) 200 mM KOH, 50 mM dithiotheitol, heat to 65 ° C for 10 min; 3) for 4 μL protease-based lysis buffer: 1 μL of 17 μM SDS combined with 3 μL of 125 μg/mL proteinase K, followed by incubation at 37 ° C for 60 min, then 95 ° C for 15 min (to inactivate the proteinase K); 4) for 10 μL of a detergent-based lysis buffer: 2 μL H₂O, 2 μL 10 mM EDTA, 2 μL 250 mM dithiothreitol, 2 μL 0.5% N-laurylsarcosin salt solution; 5) 200 mM Tris pH7.5, 20 mM EDTA, 2% sarcoyl, 6% Ficoll.

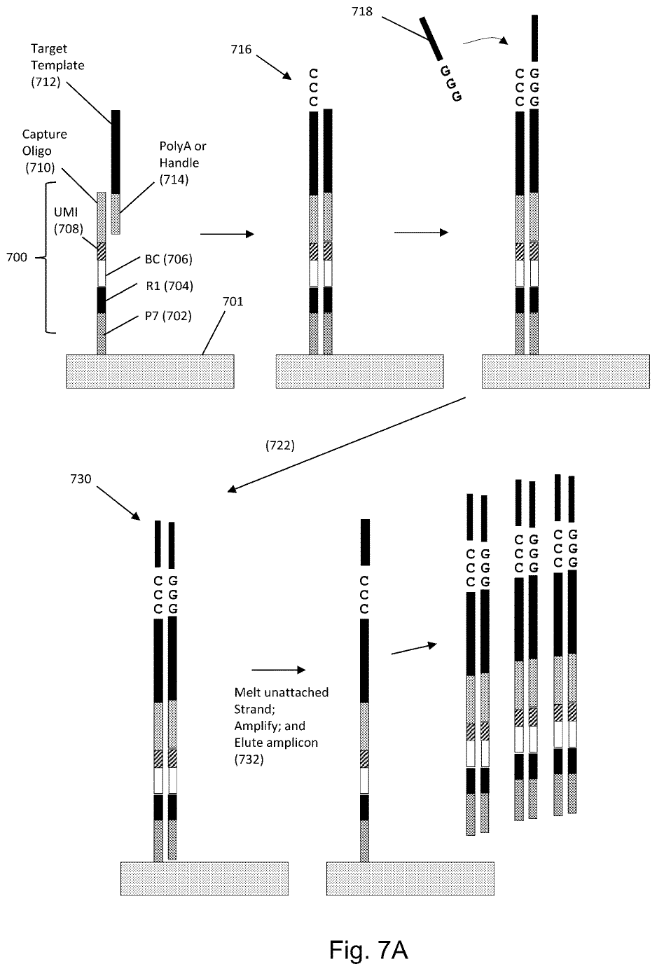

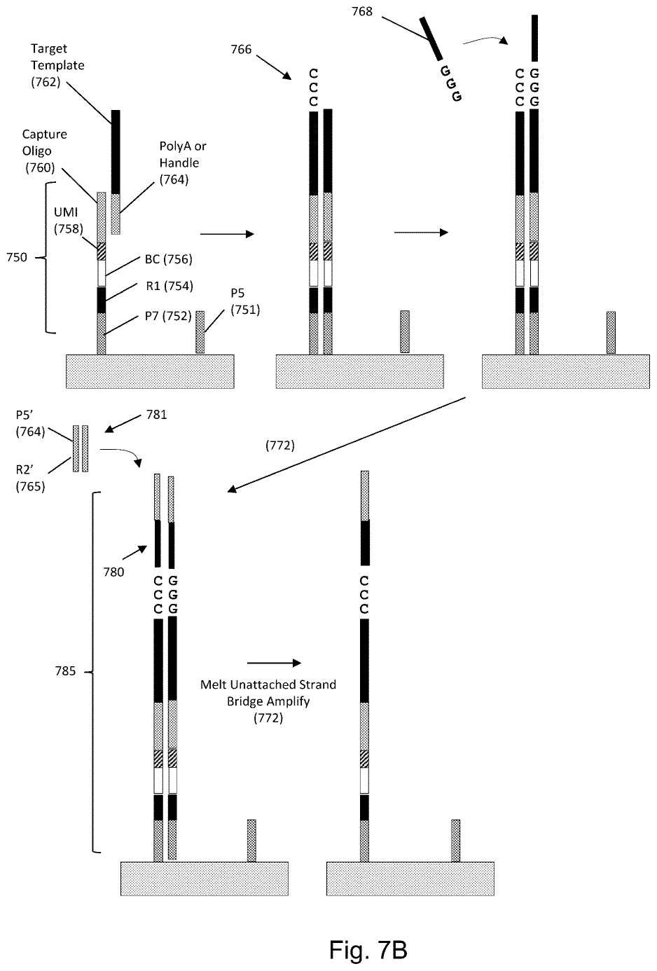

FIGS. 7A-7B illustrate example capture and cDNA synthesis methods for carrying out sequencing operations. FIG. 7A illustrates one process from capturing a target template and preparing cDNAs for external sequencing. One skilled in the art would recognize that the details of the following examples of target template capture and cDNA synthesis may vary widely depending on the sequencing system employed. In some embodiments, preparation of cDNAs includes a tagmentation step. Guidance for particular embodiments may be found in Picelli et al, Genome Research, 24:2033-2040 (2014); Bose et al, Genome Biology, 16:120 (2015); Hashimshony et al, Genome Biology, 17:77 (2016); Yuan et al, Scientific Reports, 6: 33883 (2016); and like references. Attached to surface (701) by their 5′ ends are oligonucleotides with the following components: primer binding site P7 (for Illumina sequencers) (702), optional primer binding site R1 (for Illumina paired end sequencing), barcode oligonucleotide (706) (which may be or include a spatial barcode), optional unique molecular identifier (708), and capture oligonucleotide (710), which may be a polyT segment whenever mRNA is to be captured. Target template (712) is captured by the hybridization of polyA segment or sequence handle (714) to capture oligonucleotide (710). After capture, capture oligonucleotide (710) and polyA segment (714) are extended by a polymerase (e.g. Moloney murine leukemia virus (MMLV) reverse transcriptase) that leaves a single stranded polyC tail (716). In some embodiments, template switching oligonucleotide (718) is hybridized thereto and the polyC tail is further extended, as show in (730), e.g. Zhu et al, Biotechniques, 30:892-897 (2001). The unattached strand is melted, the attached strand is amplified, e.g. by a PCR, and eluted for external sequencing (732). For in situ sequencing, surface (753) comprises attached P5 primers (Illumina) and capture oligonucleotides (750) that may have the same structure as for external sequencing: primer binding site P7 (for Illumina sequencers) (752), optional primer binding site R1 (for Illumina paired end sequencing), barcode oligonucleotide (756) (which may be or include a spatial barcode), optional unique molecular identifier (758), and capture oligonucleotide (760), which may be a polyT segment whenever mRNA is to be captured. Target template (762) is captured by the hybridization of polyA segement or sequence handle (764) to capture oligonucleotide (710). After capture, capture oligonucleotide (710) and polyA segment (764) are extended by a polymerase that leaves a single stranded polyC tail (766). In some embodiments, template switching oligonucleotide (768) is hybridized thereto and the polyC tail is further extended, as show in (780), after which double stranded segment (781) is ligated thereto. Segment (781) comprises complement R2′ (765) to primer binding site R2 and complement P5′ to primer binding site P5. The unattached strand of resulting polynucleotide (785) melted and the attached strand of (785) is bridge amplified. In some embodiments, the DNA of the resulting clusters may be sequenced using a sequencing-by-synthesis technique that generates sequences of fluorescent signals for nucleotide identification. It is understood that there are many alternatives to above process steps that may be substituted for or added to by other process steps while still being within the purview of the systems and methods described herein.

In embodiments employing spatial barcodes on a surface, a wide variety of methods may be used to generated spatial barcodes including, but not limited to, the methods described in the following references which are incorporated by reference: Horgan et al, International patent publication WO2022/013094; Fan et al, U.S. patent publication US2019/0360121; Chen et al, bioRxiv (https://doi.org/10.1101/2021.01.17.427004); Cho et al, bioRxiv (https://doi.org/10.1101/2021.01.25.427807); Quan et al, Nature Biotechnology,29 (5): 449-453 (2011); Singh-Gasson et al, Nature Biotechnology, 17:974-(1999); and the like.

In some embodiments, hydrogel chambers may be synthesized for single cells on a first surface by the following steps: (a) providing a fluidic device having (i) a channel comprising a first surface, a biological sample comprising biological cells disposed on or adjacent to the first surface, (ii) a spatial energy modulating element in optical communication with the first surface, and (iii) a detector that identifies positions of the one or more biological components in the channel based on one or more optical signals therefrom; and (b) synthesizing one or more chambers in the channel enclosing each of biological cells by projecting light into the channel with the spatial energy modulating element such that the projected light causes cross-linking of the one or more polymer precursors to form polymer matrix walls of the chambers, wherein the position of each of the synthesized chambers is determined by the position of a biological cell enclosed thereby identified by the detector. In some embodiments, the first surface comprises capture elements, such as, capture oligonucleotides covalently attached to the first surface. Example capture oligonucleotides are described in FIGS. 7A-7B. Transcriptome sequencing may be carried out with the following additional steps: (i) loading into the channel a lysing reagent that ruptures cell membranes so that messenger RNA (mRNA) is released and captured by the capture elements; (ii) loading the channel with transcription reagents to copy the captured mRNAs to produce complementary DNAs thereof; and (iii) sequencing the complementary DNAs to identify said captured mRNAs. In some embodiments, wash steps may be included, for example, to remove lysing reagents prior to loading transcription reagents, or to remove transcription reagents prior to loading amplification reagents, or to remove amplification reagents prior to loading sequencing reagents, and so on. In some embodiments, reverse transcription reagents include a reverse transcriptase. In some embodiments such reverse transcriptase is an MMLV reverse transcriptase. As mentioned above, cDNA sequencing may be carried out “in situ” or external to the system. In situ sequencing may be carried out by the following additional steps: (a) amplifying the complementary DNAs, (b) sequencing the amplified complementary DNAs, for example, using a sequencing-by-synthesis technique. After sequencing, relative expression levels of the mRNAs may be determined, thereby providing a transcriptome. In some embodiments, external, or off-channel sequencing requires that the capture elements comprise spatial barcodes as described in the above example. cDNAs may be optionally amplified after which they are eluted and sequenced by an external sequencing instrument.

Hydrogel Function. A wide variety of photosynthesizable gels may be used in connection with the systems and methods described herein. In some embodiments, hydrogels are used with the systems and methods described herein in particular because of their compatibility with living cells and the versatility of formulating gels with desired properties including, but not limited to, porosity (which in large part determines what is contained and what is passed by a gel (or polymer matrix) wall, degradability, mechanical strength, ease and speed of synthesis, and the like.

Hydrogel Porosity. In some embodiments, hydrogel porosity is selected to permit passage of selected reagents while at the same time preventing the passage of other reagents or objects, such as, a cell. In some embodiments, hydrogel porosity is selected to prevent the passage of biological cells but to permit the passage of reagents, including proteins, such as polymerases. In some embodiments, such reagents permeable to a polymer matrix wall comprise lysozyme, proteinase K, random hexamers, polymerases, transposases, ligases, deoxynucleotide triphosphates, buffers, cell culture media, or divalent cations. In some embodiments, the at least one polymer matrix comprises pores that are sized to allow diffusion of a reagent through the at least one polymer matrix but are too small to allow DNA or RNA for analysis to traverse the pores (having a size of greater than 100 nucleotides or basepairs, or greater than 300 nucleotides or basepairs). In some embodiments, crosslinking the polymer chains of the hydrogel structure forms a hydrogel matrix having pores (i.e., a porous hydrogel matrix). In some versions, the size of the pores in the hydrogel structures may be regulated or tuned and may be formulated to encapsulate sufficiently large genetic material, such as cells or nucleic acids (e.g., of greater than about 300 base pairs), but to allow smaller materials, such as reagents, or smaller sized nucleic acids (e.g., of less than about 50 base pairs), such as primers, to pass through the pores, thereby passing in and out of the hydrogel structures. In some embodiments, the hydrogels can have any pore size having a diameter sufficient to allow diffusion of the above-listed reagents through the structure while retaining the nucleic acid molecules greater than 500 nucleotides or basepairs in length. In some embodiments, the hydrogel structure can be swollen when the hydrogel is hydrated. The sizes of the pores can then change depending on the water content in the hydrogel of the hydrogel structure. In some embodiments, the pores have a diameter of from about 10 nm to about 100 nm. In some embodiments, the pore size of the hydrogel structures is tuned by varying the ratio of the concentrations of polymer precursors to the concentration of crosslinkers, varying pH, salt concentrations, temperature, light intensity, and the like, by routine experimentation. In some embodiments, the average diameter of pores of a polymer matrix wall prevent passage of molecules having a molecular weight of 25 kiloDaltons (kDa) or greater; or having a molecular weight of 50 kDa or greater; or having a molecular weight of 75 kDa or greater; or having a molecular weight of 100 kDa or greater; or having a molecular weight of 150 kDa or greater.

In some embodiments, DNA or RNA retained have lengths that are sequencable using conventional sequencing-by-synthesis techniques. For example, such DNA or RNA comprise at least 50 nucleotides, or in some embodiments, at least 100 nucleotides. In some embodiments, the pores may have an average diameter from 5 nm to 100 nm. In some embodiments, the pores may have an average diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have an average diameter larger than 100 nm. In some embodiments, the pores may have an average diameter smaller than 5 nm.

The reagent may comprise an enzyme or a primer having a size of less than 50 base pairs (bp). A primer may comprise a single-stranded DNA (ssDNA). In some embodiments, a primer may have a size from 5 bp to 50 bp. In some embodiments, a primer may have a size from 5 bp to 10 bp, 10 bp to 20 bp, from 20 bp to 30 bp, 30 bp to 40 bp, or 40 bp to 50 bp. In some embodiments, a primer may have a size of more than 50 bp. In certain cases, a primer may have a size of less than 5 bp. In some embodiments, the pores may have a diameter from 5 nm to 100 nm. In some embodiments, the pores may have a diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have a diameter larger than 100 nm. In some embodiments, the pores may have an average diameter smaller than 5 nm. The polymer matrix may have a pore size of about 5 nanometers (nm) to about 100 nm. The polymer matrix may have a pore size of about 5 nm to about 10 nm, about 5 nm to about 20 nm, about 5 nm to about 30 nm, about 5 nm to about 40 nm, about 5 nm to about 50 nm, about 5 nm to about 60 nm, about 5 nm to about 70 nm, about 5 nm to about 80 nm, about 5 nm to about 90 nm, about 5 nm to about 100 nm, about 5 nm to about 110 nm, about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 10 nm to about 110 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 20 nm to about 110 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 30 nm to about 110 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 40 nm to about 110 nm, about 50 nm to about 60 nm, about 50 nm to about 70 nm, about 50 nm to about 80 nm, about 50 nm to about 90 nm, about 50 nm to about 100 nm, about 50 nm to about 110 nm, about 60 nm to about 70 nm, about 60 nm to about 80 nm, about 60 nm to about 90 nm, about 60 nm to about 100 nm, about 60 nm to about 110 nm, about 70 nm to about 80 nm, about 70 nm to about 90 nm, about 70 nm to about 100 nm, about 70 nm to about 110 nm, about 80 nm to about 90 nm, about 80 nm to about 100 nm, about 80 nm to about 110 nm, about 90 nm to about 100 nm, about 90 nm to about 110 nm, or about 100 nm to about 110 nm. The polymer matrix may have a pore size of about 5 nm, about 10 nm, about 20 nm, about 30 nm, about 40 nm, about 50 nm, about 60 nm, about 70 nm, about 80 nm, about 90 nm, about 100 nm, or about 110 nm. The polymer matrix may have a pore size of at least about 5 nm, about 10 nm, about 20 nm, about 30 nm, about 40 nm, about 50 nm, about 60 nm, about 70 nm, about 80 nm, about 90 nm, about 100 nm, or less. The polymer matrix may have a pore size of at most about 10 nm, about 20 nm, about 30 nm, about 40 nm, about 50 nm, about 60 nm, about 70 nm, about 80 nm, about 90 nm, about 100 nm, about 110 nm, or more.

Modulation of Hydrogel Porosity. The pore size in the polymer matrix may be modulated using a chemical reagent, or by applying heat, electrical field, light, or another suitable stimulus. In other words, the polymer matrix may comprise tunable properties (e.g., the pore size) In some cases, the polymer matrix may comprise a thermoresponsive or temperature-responsive polymer. A thermoresponsive polymer (e.g., poly(N-isopropylacrylamide) (NIPAAM)) may phase separate from a solution upon heating or upon cooling (e.g., polymer showing lower critical solution temperature (LCST) or upper critical solution temperature (UCST). The polymer matrix may comprise polymer which may collapse at high temperature in order to, for example, control the pore size of the hydrogel or polymer matrix. Non-limiting examples of thermoresponsive polymers that may be used to form hydrogel/polymer matrix with tunable properties may include Poly(N-vinyl caprolactam), Poly(N-ethyl oxazoline), Poly(methyl vinyl ether), Poly(acrylic acid-coacrylamide), or a combination thereof. A change in temperature may enlarge or contract average pore size in the polymer matrix to allow selected molecules, such as a nucleic acid molecule, a protein, or any biomolecule or molecule smaller than the adjusted pore size to be released from a hydrogel chamber.

Size and Shape of Hydrogel Chambers. In some embodiments, a polymer matrix wall of a chamber inhibits passage of a predetermined component, such as a mammalian cell, genomic DNA, larger polynucleotides (e.g. mRNA greater than 200 ribonucleotides, or greater than 300 ribonucleotides, or 500 ribonucleotides), or the like. In some embodiments, a polymer matrix wall extends from the first surface to a second surface (parallel to the first surface) to form a chamber within a channel. In some embodiments, a chamber has polymer matrix walls and an interior. In some embodiments, the interior of a chamber is sized for enclosing a cell. For example, such chamber may comprise a cylindrical shell or a polygon shell, comprising an inner space, or interior and a polymer matrix wall. In some embodiments, such chambers have annular-like cross-sections. As used herein, the term “annular-like cross-section” means a cross-section topologically equivalent to an annulus. In some embodiments, the inner space, or interior, of a chamber has an inner diameter from 1 μm to 500 μm and a volume in the range of from 1 pico liter to 200 nano liters, or from 100 pico liters to 100 nano liters, or from 100 picoliters to 10 nano liters. In some embodiments, the polymer matrix wall has a thickness of at least 1 μm (micrometer). In some embodiments, the height of a chamber with an annular-like cross section have a value in the range of from 10 μm to 500 μm, or in the range of from 50 μm to 250 μm. In some embodiments, a polymer matrix wall having an annular-like cross-section has an aspect ratio (i.e., height/width) of 1 or less. In some embodiments, aspect ratio and polymer matrix wall thickness are selected to maximize chamber stability against forces, such as reagent flow through the channel, washings, and the like. In some embodiments, the at least one polymer matrix wall is a hydrogel wall. In some embodiments, the at least one polymer matrix is degradable. In some embodiments, the degradation of the at least one polymer matrix is “on demand.” In some embodiments, chambers in a channel are non-contiguous. In some embodiments, chambers in a channel may be contiguous with adjacent chambers. In some embodiments, chambers may share polymer matrix walls with one another. In some embodiments, chambers may be synthesized with slits or other orifaces large enough to permit passage of certain components, e.g. beads, but small enough to prevent passage of other components, e.g. cells.

Hydrogel Compositions. In some embodiments, a channel of a fluidic device of a system described herein comprises one or more polymer precursors for forming chambers. In some embodiments, the one or more polymer precursors comprise hydrogel precursors. Such precursors may be selected from a wide variety of compounds including, but not limited to, polyethylene glycol (PEG)-thiol, PEG-acrylate, acrylamide, N,N′-bis(acryloyl) cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or combinations or mixtures thereof. In some embodiments, the hydrogel comprises an enzymatically degradable hydrogel, PEGthiol/PEG-acrylate, acrylamide/N,N′-bis(acryloyl) cystamine (BACy), or PEG/PPO. In some embodiments, the following precursors and crosslinker may be used to form chambers with degradable polymer matrix (hydrogel) walls. Polymer precursors may be formed by using any hydrogel precursor and crosslinkers of Table 2A (columns 1 and 3, respectively). The resulting polymer matrices may be degraded with the indicated degradation agents in Table 2A (column 4).

TABLE 2A

| | | | Degradation |
|---|---|---|---|
| Precursors | Hydrogels | Crosslinkers | Agents |
| Acrylamide | Polyacrylamide | Bis-acryloyl cystamine | DTT/TCEP/THP |
| | | (structure 1) | |
| PEG-based | PEG | Bis(2-methacryloly)oxyethyl | DTT/TCEP/THP |
| acryloyl | | disulfide (structure 2) | |
| Dextran-based | Dextran | N,N′-(1,2- | NaIO4 |
| acryloyl | | Dihydroxylethylene)bis- | |
| | | acrylamide(structure 3) | |
| Polysaccharide- | Polysaccharide | Structure 4 | NaOH, |
| base | | | ethanolamine |
| acryloyl | | | DTT/TCEP/THP |
| Gelatin-base | Gelatin | Structure 5 | NaOH, |
| acryloyl | | | ethanolamine, |
| | | | nucleophilic bases |
| | | Structure 6 | NaOH, alkali, |
| | | | organic bases |
| | | Structure 7 | Acid |

TABLE 2B

| Structure | |
|---|---|
| Number | Formula |
| 1 | 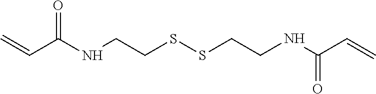 [SMILES: C=CC(=O)NCCSSCCNC(=O)C=C] |
| 2 | 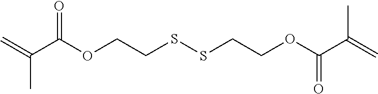 [SMILES: C=C(C)C(=O)OCCSSCCOC(=O)C(=C)C] |
| 3 | 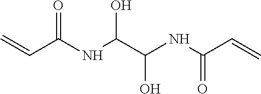 |
| 4 | 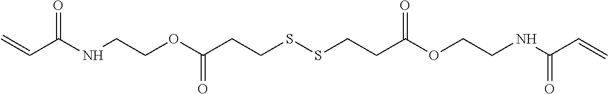 [SMILES: C=CC(=O)NCCOC(=O)CCSSCCC(=O)OCCNC(=O)C=C] |
| 5 | 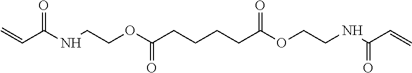 [SMILES: C=CC(=O)NCCOC(=O)CCCCC(=O)OCCNC(=O)C=C] |
| 6 | 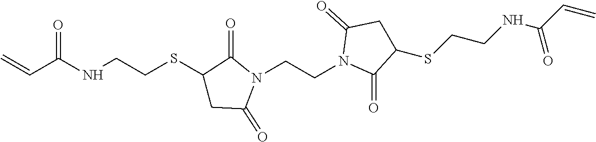 [SMILES: C=CC(=O)NCCSC1CC(=O)N(CCN2C(=O)CC(SCCNC(=O)C=C)C2=O)C1=O] |
| 7 | 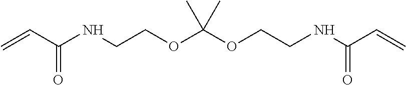 [SMILES: C=CC(=O)NCCOC(C)(C)OCCNC(=O)C=C] |
| 8 | 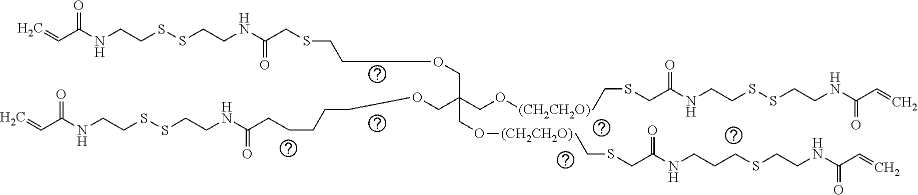 [symbol] |
| 9 | 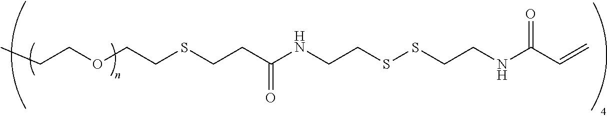 In some cases, n is an integer selected from 0-100. In some cases, n is an integer selected from 5-50. In some cases, n is an integer from 0-10. |
| 10 | 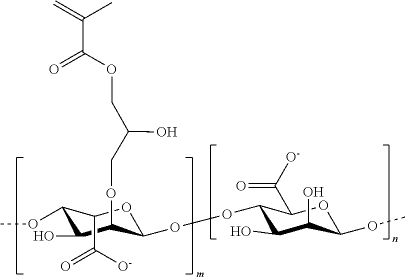 In some cases, n is an integer selected from 0-100. In some cases, n is an integer selected from 5-50. In some cases, n is an integer from 0-10. In some cases, m is an integer selected from 0-100. In some cases, m is an integer selected from 5-50. In some cases, m is an integer from 0-10. |
| 11 | 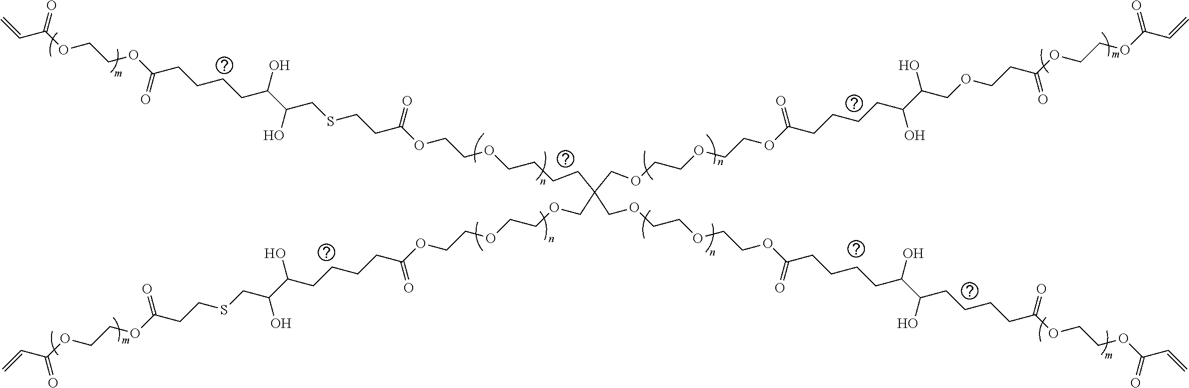 In some cases, m is an integer selected from 0-100. In some cases, m is an integer selected from 5-50. In some cases, m is an integer from 0-10. |
| 12 | 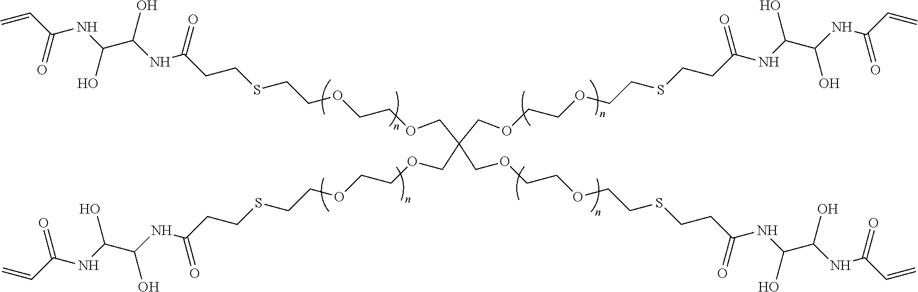 In some cases, n is an integer selected from 0-100. In some cases, n is an integer selected from 5-50. In some cases, n is an integer from 0-10. |
| 13 | 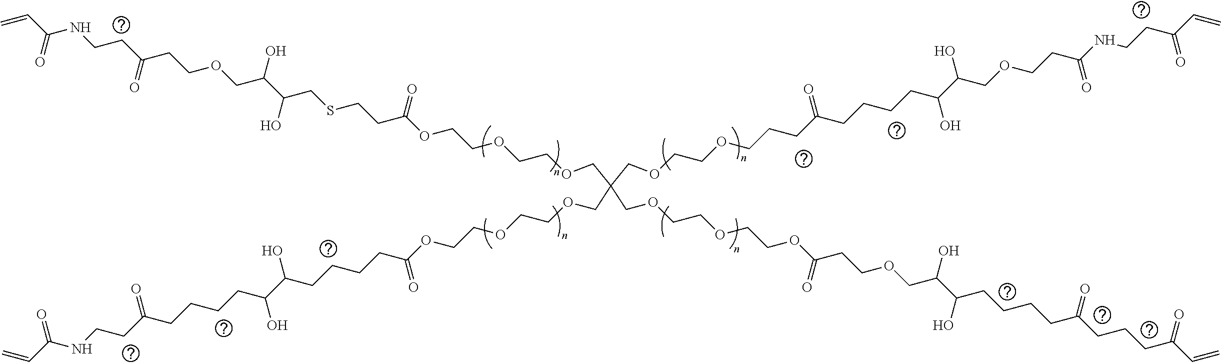 In some cases, n is an integer selected from 0-100. In some cases, n is an integer selected from 5-50. In some cases, n is an integer from 0-10. |
| 14 | 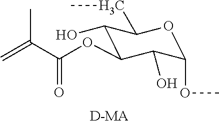 |
| 15 | 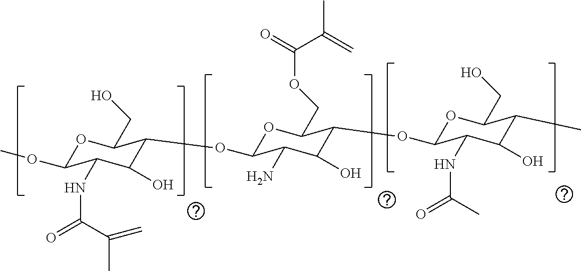 In some cases, n is an integer selected from 0-100. In some cases, n is an integer selected from 5-50. In some cases, n is an integer from 0-10. In some cases, m is an integer selected from 0-100. In some cases, m is an integer selected from 5-50. In some cases, m is an integer from 0-10. In some cases, p is an integer selected from 0-100. In some cases, p is an integer selected from 5-50. In some cases, p is an integer from 0-10. |
| 16 | 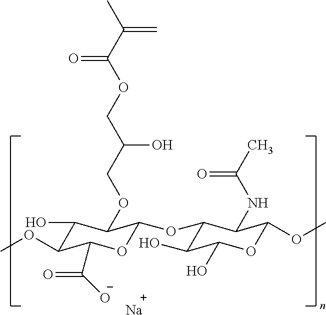 In some cases, n is an integer selected from 0-100. In some cases, n is an integer selected from 5-50. In some cases, n is an integer from 0-10. |
| 17 | 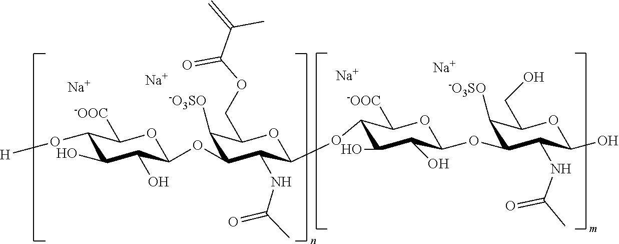 In some cases, n is an integer selected from 0-100. In some cases, n is an integer selected from 5-50. In some cases, n is an integer from 0-10. In some cases, m is an integer selected from 0-100. In some cases, m is an integer selected from 5-50. In some cases, m is an integer from 0-10. |
| 18 | 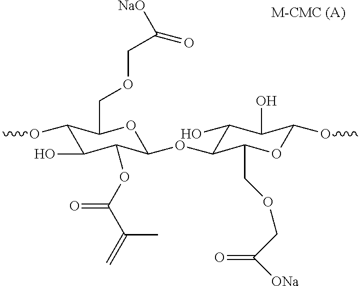 |
| 19 | 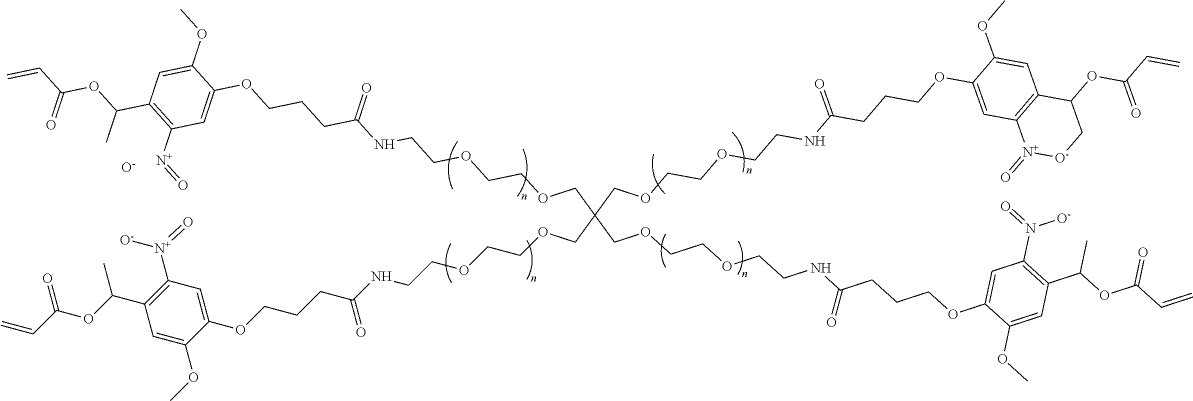 In some cases, n is an integer selected from 0-100. In some cases, n is an integer selected from 5-50. In some cases, n is an integer from 0-10. |
| 20 | 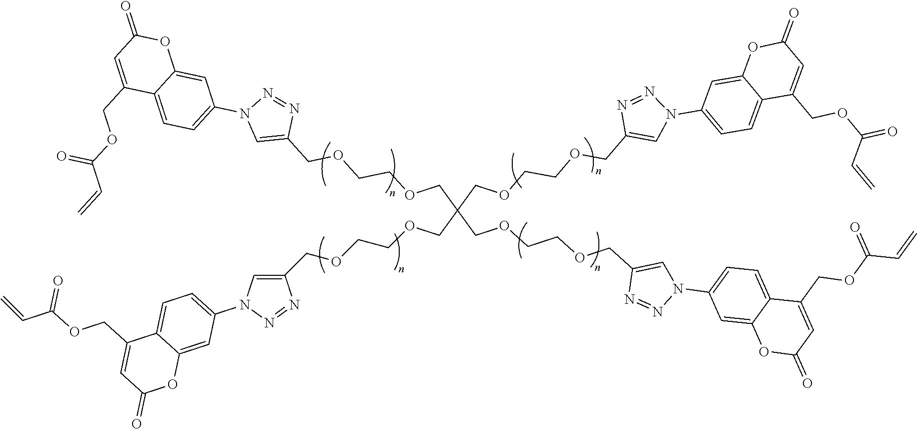 In some cases, n is an integer selected from 0-100. In some cases, n is an integer selected from 5-50. In some cases, n is an integer from 0-10. |

[symbol]

indicates data missing or illegible when filed

In some embodiments, the hydrogel chamber or the hydrogel polymer wall comprises a polymerized form of a monomer, the monomer comprising: an oligomeric domain comprising three or more arms, wherein each arm of said oligomeric domain comprises a degradable unit and a crosslinkable unit, wherein the crosslinkable unit of an arm of the three or more arms is configured to crosslink with another crosslinkable unit of another polymer precursor in response to a first stimulus, thereby obtaining the polymerized form of the monomer, and wherein the degradable unit is configured to be cleaved in response to a second stimulus, thereby solubilizing the polymerized form of the monomer. In some embodiments, the oligomeric domain comprises four or more arms. In some embodiments, the hydrogel chamber or the hydrogel polymer wall comprises a degradable functional group. In some embodiments, said degradable function group comprises disulfide, Beta-thioether ester, Amidomethylol and vicinal diol, Vicinal diol, Alginate backbone, Dextran backbone, Chitosan backbone, Hyaluronic acid backbone, Chondroitin sulfate backbone, or Carboxy methyl cellulose backbone, or a combination thereof. In some embodiments, the hydrogel chamber or the hydrogel polymer wall comprises a polymerized form of a hydrogel macromonomer. In some embodiments, the hydrogel macromonomer comprises cPEG, cSEL-BTEEC, cSEL-DHEBA, cSEL-diol, cSEL-alginate, cSEL-dextran, cSEL-chitosan, cSEL-hyaluronic acid, cSEL-chondroitin sulfate, or cSEL-cellulose, or a combination thereof. In some embodiments, the degradation unit is degraded by inputting a degradation reagent into the fluidic device. In some embodiments, the degradation reagent comprises DTT, TCEP, BME, GSH, DMEM, RPMI, PBS buffer, DMEM, RPMI, PBS buffer, sodium (meta) periodate, Alginate lyase (enzyme), Dextranase, Lysozyme and chitinase, Hyaluronidase, Chondroitinase, or Cellulases, or a combination thereof. In some embodiments, the hydrogel chamber or the hydrogel polymer wall comprises at least one beta-thioether ester. In some embodiments, the hydrogel chamber or the hydrogel polymer wall comprises a PEG-macromonomer containing beta-thioether esters. In some embodiments, the beta-thioether ester is formed by reacting an acrylate with a thiol. In some embodiments, the hydrogel chamber or the hydrogel polymer wall comprises a Michael donor. In some embodiments, the Michael donor is PEG-thiol. In some embodiments, the hydrogel chamber or the hydrogel polymer wall comprises a cSEL beta-thioether ester with one beta-thioether ester per arm. In some embodiments, the hydrogel chamber or the hydrogel polymer wall comprises are formed from any material that comprises a PEG with a Michael acceptor chain. In some embodiments, the Michael acceptor chain comprises PEG-acrylamide, PEG-vinyl sulfone, PEG-maleimide, or PEG-carbonyl acrylic, or any combination thereof. In some embodiments, the hydrogel chamber or the hydrogel polymer wall is degradable by cleavage of disulfide bonds. In some embodiments, the disulfide bonds are cleavable by one or more reducing agents. In some embodiments, the one or more reducing agents comprise DTT, TCEP, BME, or GSH, or any combination thereof. In some embodiments, the hydrogel chamber or the hydrogel polymer wall comprises one or more arms each comprising one or more amides.

Hydrogel Degradation. In some embodiments, hydrogel chambers are degradable or depolymerizable either generally within a channel or “on demand” within a channel. Hydrogel chambers that are generally degradable are degraded by treatment with a degradation agent, or equivalently, a depolymerization agent that is exposed to all chambers within channel. Example depolymerization agents include, but are not limited to, heat, light, and/or chemical depolymerization reagents (also sometimes referred to a cleaving reagents or degradation reagents). In some embodiments, on demand degradation may be implemented using polymer precursors that permit photo-crosslinking and photo-degradation, for example, using different wavelengths for crosslinking and for degradation. For example, Eosin Y may be used for radical polymerization at defined regions using 500 nm wavelength, after which illumination at 380 nm can be used to cleave the cross linker. In other embodiments, photo-caged hydrogel cleaving reagents may be included in the formation of polymer matrix walls. For example, acid labile crosslinkers (such as esters, or the like) can be used to create the hydrogel and then UV light can be used to generate local acidic conditions which, in turn, degrades the hydrogel. In some embodiments, the at least one polymer matrix is degradable by at least one of: (i) contacting the at least one polymer matrix with a cleaving reagent; (ii) heating the at least one polymer matrix to at least 90 ° C.; or (iii) exposing the at least one polymer matrix to a wavelength of light that cleaves a photo-cleavable cross linker that cross links the polymer of the at least one polymer matrix. In some embodiments, the at least one polymer matrix comprises a hydrogel. In some embodiments, the cleaving reagent degrades the hydrogel. In some embodiments, the cleaving reagent comprises a reducing agent, an oxidative agent, an enzyme, a pH based cleaving reagent, or a combination thereof. In some embodiments, the cleaving reagent comprises dithiothreitol (DTT), tris(2-carboxyethyl)phosphine (TCEP), tris(3-hydroxypropyl)phosphine (THP), or a combination thereof. In some embodiments, the surface of the polymer matrix or hydro gel may be functionalized by coupling a functional group to the polymer matrix or hydrogel. Some nonlimiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized polymer matrix may be used to capture biomolecules inside a polymer matrix compartment formed adjacent to (e.g., around or on) the biological component. The biomolecule may be produced by the biological component (e.g., secretome from a cell). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

Partial Degradation. In some embodiments, existing polymer matrix walls may be partially degraded, e.g. to change porosity. In some embodiments, polymer precursors may include degradable beads that form part of, and are embedded in, the polymer matrix walls when synthesized, after which either on-demand or generally, may be degraded, thereby creating an increase in porosity.

Photosynthesis. In some embodiments, the generation of a polymer matrix within said fluidic device comprises exposing the one or more polymer precursors to an energy source. In some embodiments, the energy source is a light generating device. In some embodiments, the light generating device generates light at 350 nm to 800 nm. In some embodiments, the light generating device generates light at 350 nm to 600 nm. In some embodiments, the light generating device generates light at 350 nm to 450 nm. In some embodiments, the light generating device generates UV light. In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid-crystal based.

In some embodiments, systems described herein comprise (a) a channel comprising a first surface, a plurality of cells disposed on the first surface, and one or more polymer precursors; (b) a spatial energy modulating element in optical communication with the first surface; (c) a detector in optical communication with the first surface and in operable association with the spatial energy modulating element, the detector detecting each of the plurality of cells and determining a position thereof on the first surface; and (d) a plurality of gel chambers each gel chamber enclosing a single cell of the plurality of cells wherein the gel chambers are synthesized by projecting light into the channel with the spatial energy modulating element such that the projected light causes cross-linking of the one or more polymer precursors to form polymer matrix walls of the chambers, wherein the positions of the synthesized chambers are determined by the positions of cells enclosed thereby identified by the detector. It is understood that the term “detector” as used herein may include, but not be limited by, a microscope element that collects and optionally magnifies an image of a portion of a channel and an image analysis element that comprises software for identifying cells, cellular features, chambers, and other objects, for storing such information as well as associated position information. A computer element uses such information generated by a detector together with user input to generate commands to other elements, such as, the spatial energy modulating element to carry out a variety of functions including, but not limited to, synthesizing chambers, “on-demand” degrading of chambers, photo-lysing cells, and the like. Example configurations of such embodiments are illustrated in FIGS. 5A-5B which are described above. In some embodiments, a channel of a fluidic device further comprises a second surface wherein said first surface and the second surface are disposed opposite one another across the channel, and wherein the polymer matrix walls of the chambers extend from the first surface to the second surface to form chambers each having an interior. In some embodiments, chambers in a channel each enclose a single cell. In some embodiments both the first wall and the second wall are made of optically transmissive materials, such as, glass, plastic, or the like, and are positioned so that the first surface and second surface are substantially parallel to one another. the perpendicular distance between a first surface and a second surface may be in the range of from 10 μm to 500 μm, or in the range of from 50 μm to 250 μm. In some embodiments, the perpendicular distance between a first surface and a second surface may be in the range of from twice the average size of the cells to be analyzed to five times the average size of the cells to be analyzed.

In other embodiments, the first surface may comprise capture elements for capturing cells at predetermined locations. For example, capture elements may include, but are not limited to, capture antibodies specific for all or a subpopulation of cells. Capture elements may also include, but not be limited to, non-specific capture materials, such as, polylysine, fibronectin, treated plastics (e.g. Maxysorb™ plastic, ThermoFisher), and the like. In some embodiments, such cellular capture moieties (for example, antibodies) may be restricted to spots or reaction sites arrayed in a regular pattern on the first surface; thus, cells captured at such reaction sites may be disposed on the first surface in a regular pattern that may be more efficiently than a random disposition for chamber synthesis and/or optical signal detection. Guidance for providing surfaces with cellular capture antibodies may be found in the following references: Zhu et al, Analytica Chemica Acta, 608:186-196 (2008); Sekine et al, J. Immunol. Methods, 313 (1-2): 96-109 (2006); and the like. In some embodiments, such reaction sites or spots have diameters in the range of from 5-500 mm or in the range of from 10-1000 mm. In some embodiments, such spots or reaction sites are arranged in a rectilinear array, or are arranged in a hexagonal array. In some embodiments, such arrays of such spots or reaction sites have a density in the range of from 10 to 2500 sites/mm², or from 10 to 1000 sites/mm², or from 10 to 500 sites/mm², or from 10 to 100 sites/mm².

In some embodiments, cells may be disposed randomly on the first surface. In some embodiments, cells are disposed randomly on the first surface in a Poisson distribution. In some embodiments, in such Poisson distribution cells have a nearest neighbor distance equal to or greater 10 mm, equal to or greater 20 mm, equal to or greater 30 mm, equal to or greater 40 mm, equal to or greater 50 mm, or equal to or greater 100 mm. In some embodiments, a subset of such Poisson distributed cells are each enclosed by an annular-like shaped chamber having a diameter in the range of from 10-500 mm. In some embodiments, cells are disposed randomly on the first surface in a Poisson distribution having a density in the range of from 10 to 2500 cells/mm², or from 10 to 1000 cells/mm², or from 10 to 500 cells/mm², or from 10 to 100 cells/mm².

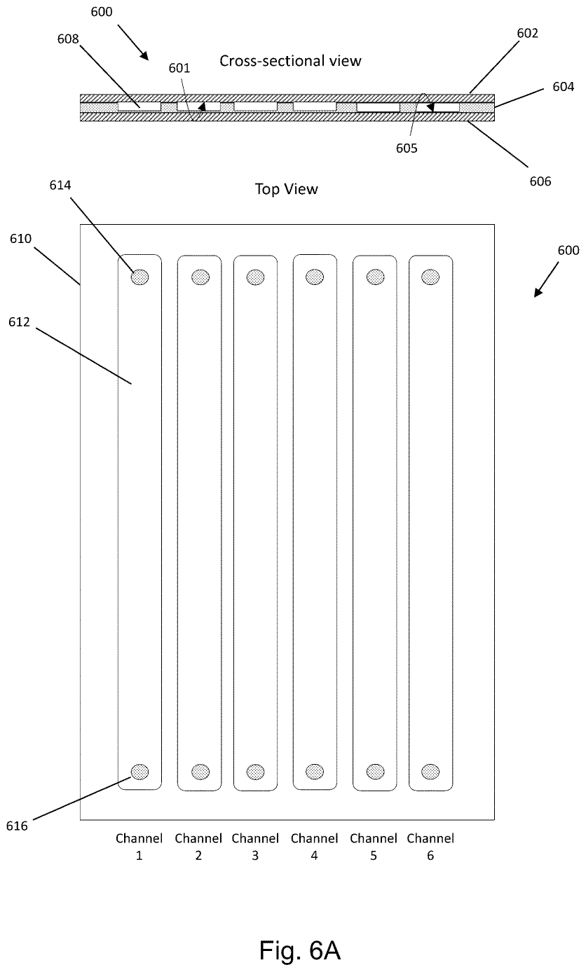

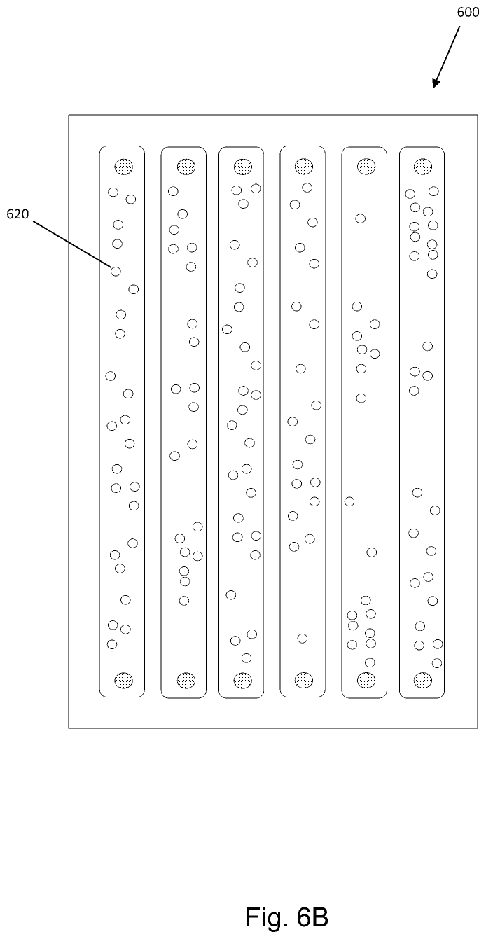

In some embodiments, a plurality of channels may be arranged together in a flow channel as illustrated in FIGS. 6A-6B. In some embodiments, the plurality of channels may be in the range of from 2 to 12, or from 2 to 8, or from 2 to 6, or in the range of from 2 to 4. Example flow cell (600) is shown in a cross-sectional view and a top view. Flow cell (600) has bottom, or first, wall (606) with first surface (605); top, or second, wall (602) with second surface (601); and sandwiched sealingly therebetween spacer (604) whose longitudinal holes form channels 1-6, one of which is indicated by (608) in the cross-sectional view, and by (612) in the top view. In some embodiments, spacer (604) may have a thickness in the range of from 10 mm to 500 mm, or in the range of from 50 mm to 250 mm, which determines the interior height of the channels. Top wall (602) comprises inlets (614) and outlets (616) for either separately or jointly loading and removing reagents and cells from channels 1-6. In some embodiments, at least one of walls (602) and (606) are made of light transmissive materials, such as glass, plastic, or the like. Flow cell (600) may be operationally associated with a fluidic device that delivers reagents and cells to any of channels 1-6 under programmed control. Guidance for particular designs, including fluid handling and valving for such fluidic systems may be found in U.S. Pat. Nos. 8,921,073; 8,173,080; 8,900,828; and the like, which are incorporated herein by reference. FIG. 6B illustrates channels of flow cell (600) with random distributions (not to scale) of hydrogel chambers with annulus-like cross-sections, such as (620), on their first surfaces.

As noted above, any of first surfaces, second surfaces or polymer matrix wall of chambers may comprise capture elements and other functional groups for carrying out a variety of operations including, but not limited to, capturing cells, capturing constituents of cells (such as, mRNA, secreted proteins, intracellular proteins, or genomic sequences), capturing constituents of analytical reagents (such as, oligonucleotide labels from antibodies), and the like. Derivatizing surfaces for such purposes is well-known to those skilled in the art, as evidenced by the following references: Zhu et al (cited above); Sekine et al (cited above); Integrated DNA Technologies brochure (cited above); Hermanson (cited above); and the like.

As noted above, in some embodiments, a fluidic device of the method comprises or is operationally associated with a detector that either may share an optical path of the spatial energy modulating element or may be disposed adjacent to the second wall or opposite the first wall from the spatial energy modulating element in embodiments, such as wells, that have only a first wall and first surface. The detector is positioned so that it is capable of detecting optical signals from or adjacent to cells in the channel, for example, distributed over the first surface in chambers. In some embodiments, the first and second walls each comprise optically transmissive material, for example, so that a spatial energy modulating element may project light energy to the interior of the channel, and so that a detector may detect optical signals, such as fluorescent emissions or reflected light from biological components. In some embodiments, the projected energy from the spatial energy modulating element is a light energy from a light beam. In some embodiments, the light beam projected by the spatial energy modulating element may have a complex cross-section that permits (in various embodiments) the simultaneous synthesis of a plurality of chambers. Optically transmissive materials include, but are not limited to, glass, quartz, plastic, and like materials. In some embodiments, the step of synthesizing chambers includes positioning the chambers so that they encapsulate the one or more biological components based on the optical signals detected by the detector. That is, in some embodiments, the detector is operationally associated with the spatial energy modulating element to selectively project one or more light beams to locations where detected optical signals indicate the presence of cell of interest. In such embodiments, the detector and spatial energy modulating element are operationally associated so that the spatial energy modulating element is configured to generate an energy beam having predetermined beam characteristics. For example, one such characteristic may be a beam cross-section which results in the biological components of interest being enclosed by annular-like shaped chambers. In such operational association, optical signals detected by the detector may include, but is not limited to, morphology of biological components, for example, cell morphology; cell motility; interaction of one cell type with another cell type, such as binding of one cell type to another cell type; a presence, absence or quantity of a label on the cell, or the like.

Spatial energy modulating elements using light energy for polymerization may comprise physical photomasks or virtual photomask, such as, a digital micromirror device (DMD). The following references, which are hereby incorporated by reference, provide guidance in selecting and operating a DMD for photopolymering gels: Chung et al, U.S. Pat. No. 10,464,307; Hribar et al, U.S. Pat. No. 10,351,819; Das et al, U.S. Pat. No. 9,561,622; Huang et al, Biomicrofluidics, 5: 034109 (2011); and the like.

Culturing of individual cells in these microstructured compartments is challenging due to the absence of cell-cell interaction & cooperative effects resulting from growth factors secreted by neighboring cells when they are proliferating. Furthermore, some assays require such as Antibody dependent cellular cytotoxicity may benefit from only exposing the target cells to secreted antibodies from secreting cells without mixing the two populations. Hence, a system for single cell assays that allows for non-contact communication of different cell types would be useful for many applications.

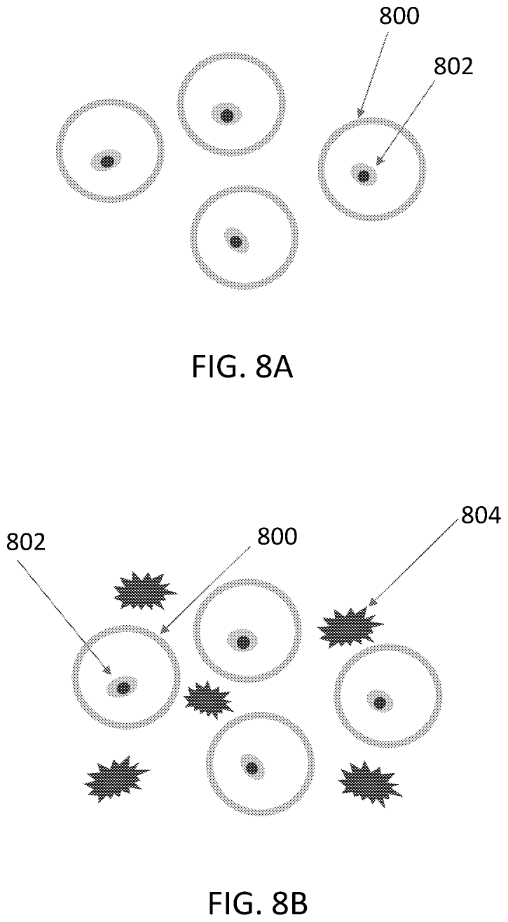

FIG. 8A shows an example of a non-activated single cells (802) captured by a plurality of hydrogel chambers (800). In various examples, the small number of cells in a hydrogel chamber can range from about 1 to about 2000 cells, preferably from about 1 to about 1000 cells, more preferably from about 1 to about 100 cells, and yet more preferably about 1 to about 10 cells. In some aspects, a chamber encloses a single cell. Non-limiting examples of cells that may be enclosed in hydrogel chambers include OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, and cancer stem cells (CSCs). The methods are also applicable to cell clusters such as organoids, neurospheres, tumors, and tumor sections. In particular embodiments, the type of cells in the hydrogel chamber can be primary cells, or more particularly OT-1 mouse primary T-cells and/or dendritic cells.

It was surprisingly determined herein that “interstitial cells”, that is cells in proximity to but disposed outside of hydrogel chambers, can improve the survival and proliferation of cells enclosed within hydrogel chambers. Interstitial cells also exhibited the ability to activate hydrogel chamber-enclosed cells. Without being bound by theory, it is hypothesized herein that interstitial cells act on chamber-enclosed cells by secreting factors that promote particular phenotypes. Accordingly, alternate embodiments provide a first cell in a first chamber and a second cell in a second chamber, or a first cell and a second cell co-encapsulated within a single chamber.

Examples of interstitial cells include activated OT1 cells, activated T cells, activated B cells, macrophages, dendritic cells, monocytes, astrocytes, microglia, stromal cells, feeder cells, cancer associated fibroblast (CAF), and tumor associated macrophages (TAM). In some cases, an interstitial cell is a cell of the same type as a chamber-enclosed cell. In particular, the interstitial cell can be an activated cell of the same type as the chamber-enclosed cell. For example, an interstitial cell may be an activated immune cell in proximity to hydrogel chambers that enclose non-activated immune cells. The activated immune cell can improve the survival of the hydrogel chamber-enclosed immune cells. The activated immune cell may also activate the non-activated immune cells enclosed within the hydrogel chambers.

Activation of the hydrogel chamber-enclosed cells may be detected by measuring surface marker expression. Examples of surface markers useful for detecting activation include CD69 (e.g., for measuring activation of T, B, and NK cells); CD71 (e.g., for measuring activation of T cells); CD25 (e.g., for measuring activation of T cells); CD107a (e.g., for measuring activation of T cells, NK cells, and macrophages); 4-1BB (e.g., for measuring activation of CD8+ T cells, CD4+ T cells, B cells, NK cells, monocytes and DCs); PD-1 (e.g., for measuring activation of T cells and NK cells); CD80, CD86 and CD83 (e.g., for measuring activation of dendritic cells); CD34 and CD133 (e.g., for measuring activation of stem cells); SSEA4 and TRA-1-60 (e.g., for measuring activation of iPSCs); EpCAM (e.g., for measuring activation of tumor cells); and CA9. Surface marker expression can be measured, for example, by inputting detectable (e.g., fluorescently labeled) antibodies into a fluidic device that contains hydrogel chamber-enclosed cells, and measuring binding of the antibodies to the cells. Cell activation may also be detected by measuring cytokine secretion (e.g., IFNg or TNFa). Cytokine secretion may be measured by capturing one or more types of cytokines on a bead (e.g., a bead functionalized with antibodies specific for the one or more cytokines), and measuring binding of detectable antibodies to cytokines bound to the bead (i.e., a bead-based ELISA assay).

Even in bulk situations (e.g., macroscale containers that do not have hydrogel chambers as described herein), OT-1 primary mouse cells are known for being vulnerable and difficult to culture. Under various circumstances, OT-1 cells can be difficult to culture in hydrogel chambers having inner dimeters around 10 to 500 microns where the cells do not survive for 48 hours or do not propagate through cell division. Surprisingly, loading pre-activated OT-1 cells in the interstitial space between hydrogel chambers with cell(s), the viability of cells inside the hydrogel chamber was dramatically improved. For instance, FIG. 8B illustrates an example of activated OT-1 cells (804) in the interstitial space amongst a plurality of hydrogel chambers containing at least one un-activated cell.

A “single-cell culture in community” concept can be applied to many types of co-culture condition, allowing tracking the single cells in specific compartmentalization while providing it with combination of other cells type. This can enhance cell growth in a cage and/or enable non-contact cell-cell interaction experiments.

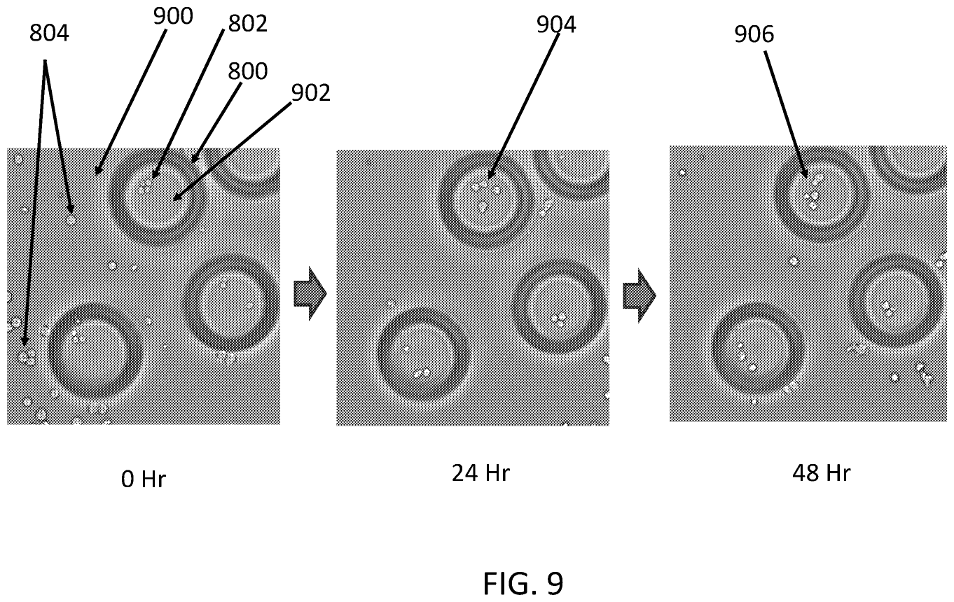

In various embodiments, a method of culturing T-cells can include loading a channel (e.g., (200)) with one or more cells and one or more polymer precursors. After identifying the location of the one or more non-activated cells (802), the one or more polymer precursors can be polymerized to form a hydrogel chamber (800) that encloses the one or more cells, wherein the hydrogel chamber is disposed within the channel. The one or more cells cannot flow through a wall of the chamber (800) from an interior of the chamber to an exterior of the chamber. However, lower molecular weight species can flow through the wall of the chamber (800) from an interior of the chamber to an exterior of the chamber where the lower molecular weight species has a size smaller than a pore or pathway cross-sectional area of the wall of the chamber. For example, a lysing agent can be permeable through the wall of the chamber (800). After polymerizing the one or more polymer precursors to form the chamber, the channel is loaded with a medium containing a component (900), as illustrated in FIG. 9 at 0 hours. In various embodiments, the medium containing a component can be a cell culture medium containing activated OT1 cells (804) that is loaded into the interstitial space after forming the hydrogel chambers. As used herein, the terms “OT-1 cells” and “OT1 cells” refer to cells derived from OT-1 transgenic mice, and in particular to CD4+ and CD8+ T cells from this mouse line. While OT-1 cells are applicable to many of the embodiments disclosed herein, it will be appreciated by those of skill in the art that other cells (e.g., immune cells and non-immune cells derived from other transgenic or non-transgenic mice or other organisms) are also suitable in the systems and methods disclosed herein.

In other embodiments, the medium containing a component can be a cell culture medium containing un-activated OT1 cells and phorbol myristate acetate (PMA). The combination of un-activated OT1 cells and PMA can result in activated OT1 cells being formed in the interstitial space in the channel. After 24 hours, as illustrated in region (904) of FIG. 9, the non-activated cells survived and became activated to grow in size. After 48 hours, as illustrated in region (906) of FIG. 9, the now activated cells continued to grow through cell division. Thus, after loading the channel with the medium, a characteristic of the chamber can be measured of the one or more cells enclosed in the chamber.

In various embodiments, the one or more cells can include T-cells, OT1 cells, mouse OT1 primary cells, or a combination thereof. In various embodiments, the component is selected from the group consisting of an activator, inhibitor, activated cell, non-activated cell, growth factor, and combinations thereof.

In various embodiments, the component comprises activated OT1 cells, wherein the activated OT1 cells cannot pass through the wall of the chamber from the exterior of the chamber to the interior of the chamber. Thus, the activated OT1 cells in the cell medium remain in the interstitial space because the activated OT1 cells are larger than the pore size of the wall of the chamber. In addition, the originally un-activated OT1 cells inside remain in an interior portion of the chamber because the originally un-activated OT1 cells are larger than the pore size of the wall of the chamber. With respect to FIG. 9, the originally un-activated OT1 cells inside the chamber cannot physically contact or touch the activated OT1 cells outside of the chamber.

In various embodiments, the activated OT1 cells secrete a signaling chemical that interacts with the one or more cells within the chamber to cause an increased cell life and cell division compared to a situation where there is an absence of the activated OT1 cells proximate to the exterior of the chamber. The signaling chemical can be a growth factor generated by the activated cells disposed outside of the chamber. The growth factor can have a size sufficiently small so that it can permeate through a wall of the chamber to the interior portion. In various embodiments, the component secretes a signaling chemical that interacts with the one or more cells within the chamber, wherein the signaling chemical permeates through a wall of the chamber to the interior portion.

In various embodiments, the characteristic can be one or more of (i) a change in total number of cells during a time duration, (ii) an activation of the one or more cells during the time duration, (iii) a change in the total number of live cells during the time duration, (iv) a change in the total number of dead cells during the time duration, and (v) combinations thereof. In other embodiments, the measuring a characteristic can be a test as noted in FIG. 1A or a single cell assay as noted in FIG. 1B (e.g., 1-20).

The un-activated OT1 cells in the chamber can become activated and then produce CD71 antigen. In certain embodiments, CD71 antibodies coupled to phycoerythrin dye (PE) day can flow through hydrogel chambers. As such, when the unactivated OT1 cell becomes activated, the CD71 antibody can bind to the expressed CD71 antigen to provide a fluorescent signal from the bound PE dye. The fluorescent signal can be generated based on the amount of CD71 antigen that is expressed.

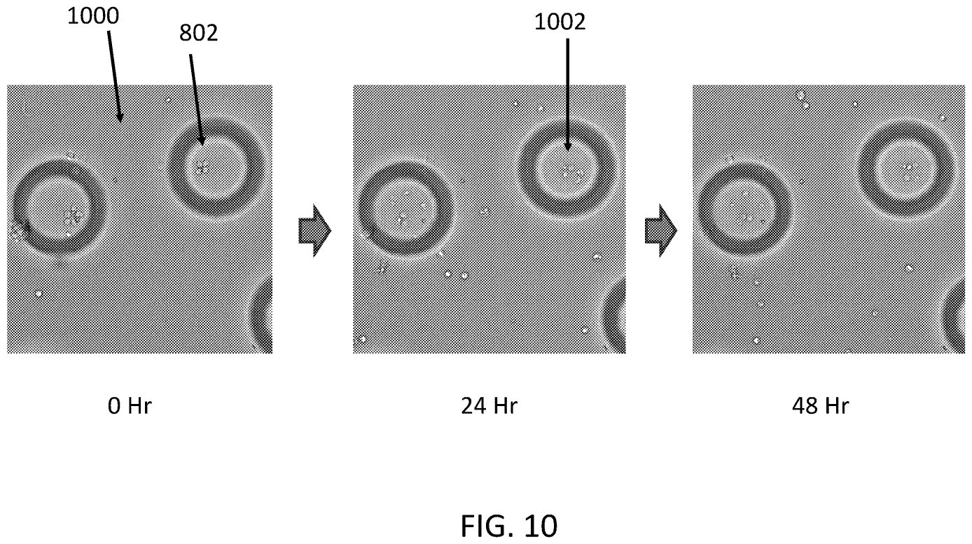

FIG. 10 illustrates the culturing of un-activated OT1 cells (802) without activated cells in the interstitial space. The interstitial space is loaded with cell culture media (1000) that contains PMA. It should be noted that there is a relatively low amount of non-activated cells in the interstitial space in FIG. 10 that is much less than the amount of activated cells in the interstitial space in FIG. 9. After 24 hours, as illustrated in region (1002) of FIG. 10, the non-activated cells died, shrinked in size, and formed a wrinkled surface. After 48 hours, as illustrated in FIG. 10, the cell viability inside of the hydrogel chamber did not improve. FIG. 9 and FIG. 10 indicate that the presence of the activated cells in the interstitial space improved cell viability inside of the hydrogel chambers and also that the presence of PMA in the interstitial space without activated cells (or at least a relatively low amount of activated cells) did not improve the cell viability inside of the hydrogel chamber.

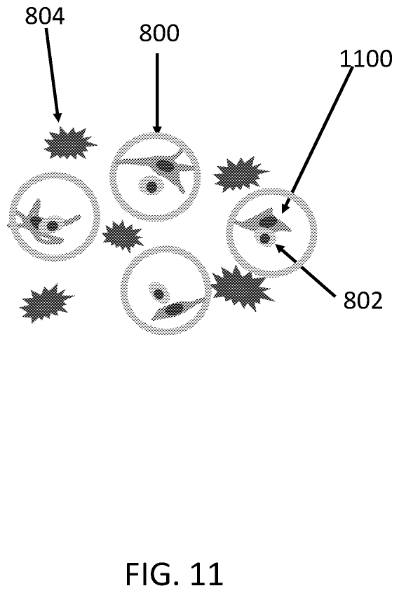

FIG. 11 shows an example of a non-activated single cell (802) and a dendritic cell (1100) captured by a plurality of hydrogel chambers (800). In addition, FIG. 11 illustrates activated OT-1 cells (804) in the interstitial space amongst a plurality of hydrogel chambers (800).

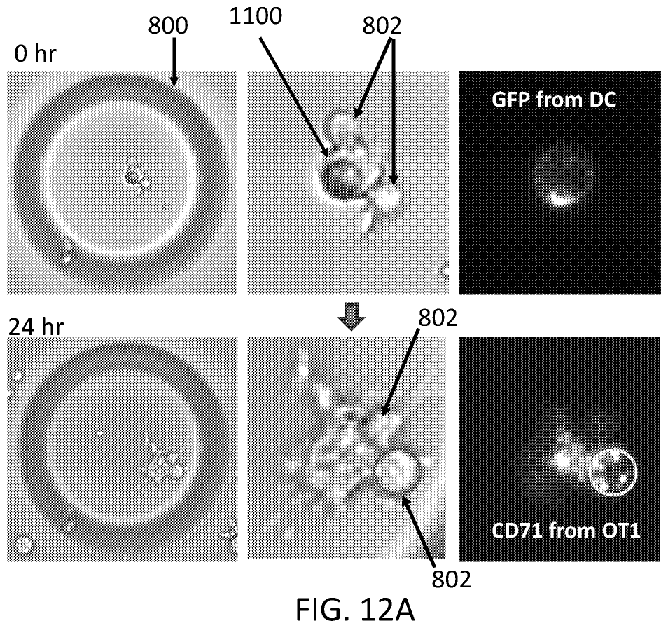

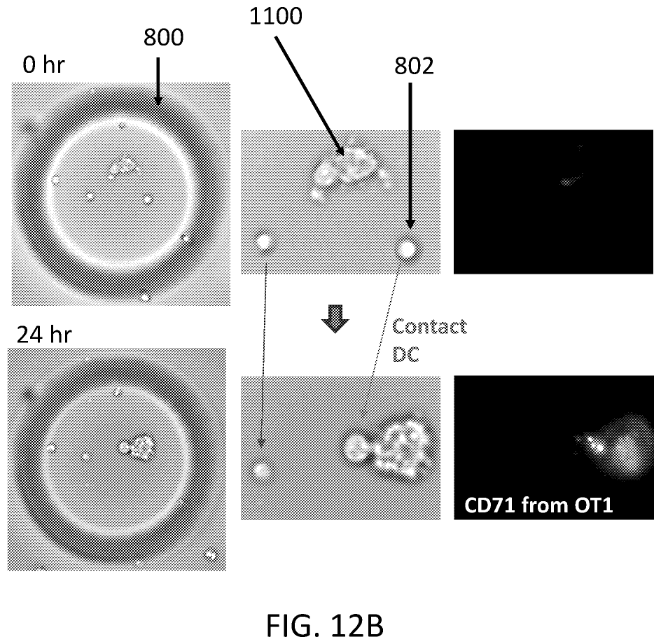

In various embodiments, a method of co-culturing cells can include loading a channel (e.g., (200)) with one or more cells of a first type, one or more cells of a second type, and one or more polymer precursors. After identifying the location of the one or more non-activated cells (802) the one or more dendritic cells (1100), the one or more polymer precursors can be polymerized to form a hydrogel chamber (800) that encloses the one or more non-activated cells (802) and the one or more dendritic cells (1100), wherein the hydrogel chamber is disposed within the channel. In many instances, the non-activated cell (802) and the dendritic cell (1100) will be proximate to each other such as, for example, within separation distance of 250 microns or less. The non-activated cells and dendritic cells cannot flow through a wall of the chamber (800) from an interior of the chamber to an exterior of the chamber. However, lower molecular weight species can flow through the wall of the chamber (800) from an interior of the chamber to an exterior of the chamber (and vice versa) where the lower molecular weight species has a size smaller than a pore or pathway cross-sectional area of the wall of the chamber. For example, a lysing agent can be permeable through the wall of the chamber (800). After polymerizing the one or more polymer precursors to form the chamber, the channel is loaded with a medium containing activated cells. After 24 hours, as illustrated in FIG. 12A (far left-hand side column), the non-activated cells survived, physically interacted with the dendritic cell, and became activated to grow in size. FIG. 12A (middle column) shows an exploded view of the cells based on FIG. 12A (far left-hand side column). FIG. 12A (far right-hand side column) shows fluorescent imaging of the dendritic cells that are genetically configured to produce green fluorescent protein and the OT1 cells that produce CD71 antigen markers when the OT1 is activated. A CD71 antibody coupled with PE dye is flowed into the hydrogel chambers to bind to the CD71 antigen to create the fluorescent signal. Thus, FIG. 12A indicates that that OT1 and dendritic cells are viable when co-cultured with activated cells in the interstitial space.

Similar to FIG. 12A, FIG. 12B shows non-activated OT1 cells (802) and dendritic cells (1100) enclosed in a chamber (800). However, at time 0 hours, non-activated OT1 cells (802) and dendritic cells (1100) do not touch each other. After 24 hours, as illustrated in FIG. 12B (middle column), the non-activated cells survived and physically moved to make contact with the dendritic cell, and then became activated to grow in size. FIG. 12B (far right-hand side column) shows fluorescent imaging of the OT1 and dendritic cells based on the green fluorescent protein labeling and CD71 marker production form the OT1 cells. Thus, FIG. 12B also indicates that that OT1 and dendritic cells are viable when co-cultured with activated cells in the interstitial space.

In various embodiments, the one or more cells of the first type include antigen receiving cells, T-cells, or OT1 cells. In various embodiments, the one or more cells of the first type include antigen receiving cells or dendritic cells. In various embodiments, the component is selected from the group consisting of an activator, inhibitor, activated cell, non-activated cell, growth factor, and combinations thereof.

In various embodiments, the component includes activated OT1 cells, wherein the activated OT1 cells cannot pass through the wall of the chamber from the exterior of the chamber to the interior of the chamber.

In various embodiments, the activated OT1 cells secrete a signaling chemical that interacts with the one or more cells of the first type or the second type within the chamber to have an increased cell life compared to a situation where there is an absence of the activated OT1 cells proximate to the exterior of the chamber. The signaling chemical can be a growth factor.

In various embodiments, the one or more cells of the first type physically contacts with the one or more cells of the second type within the chamber to form an activated state of either the one or more cells of the first type and/or the one or more cells of the second type. For example, the dendritic cells are configured to produce GFP and the OT1 cells are in an activated state, wherein the activated OT1 cell in the chamber produce CD71 antigen. A CD71 antibody coupled to a PE dye can be flowed into the hydrogel chamber and bind to the CD71 antigen creating a fluorescent signal providing spatial and quantitative data on the amount of CD71 antigen in the chamber.

In various embodiments, a method of analyzing cells includes loading a channel with (i) one or more cells of a first type, (ii) one or more cells of a second type, and (iii) one or more polymer precursors. The one or more polymer precursors can be polymerized to form a first chamber that encloses the one or more cells of the first type within the channel. The one or more polymer precursors can be polymerized to form a second chamber that encloses the one or more cells of the second type within the channel. The one or more cells of the first type cannot flow through a wall of the first chamber from an interior of the first chamber to an exterior of the first chamber. The one or more cells of the second type cannot flow through a wall of the second chamber from an interior of the second chamber to an exterior of the second chamber. After the polymerizing the one or more polymer precursors to form the first chamber and the second chamber, the channel can be loaded with a medium containing a component. After loading the channel with the medium, a characteristic can be measured of the one or more cells of the first type in the first chamber and/or the one or more cells of a second type in the second chamber.

In various embodiments, the first chamber and the second chamber are disposed in a spaced apart arrangement having a separation distance ranging from 5 to 1000 microns. The first chamber and the second chamber can each have an inner diameter ranging from 10 to 500 microns. The second chamber can have an inner diameter that is 2 to 10 times greater than an inner diameter of the first chamber.

In an alternative embodiment of analyzing cells, the method includes flowing one or more cells of a first type into a channel, wherein the channel comprises a fluidic structure configured to trap the flowing one or more cells of the first type at the fluidic structure. The one or more cells of a second type are flowed into the channel along with a medium and the one or more cells of the second type is not trapped by the fluidic structure. A characteristic of the chamber is measured of the one or more cells of a first type and/or one or more cells of a second type.

In various embodiments, the first fluidic structure is configured to at least partially enclose the one or more cells of the first type. In other embodiments, the first fluidic structure comprises a C-shaped structure to trap the one or more cells of the first type. In yet other embodiments, the first fluidic structure comprises an array of pillars configured to trap the one or more cells of the first type.

In various embodiments, the channel includes a first surface and second surface, wherein in an axis perpendicular to the first surface and/or the second surface, the fluidic structure does not touch both the first surface and second surface (e.g., the fluidic structure touches only the first surface and not the second surface, or only the second surface and not the first surface). The fluidic structure may include a non-porous polymer material. Alternatively, the fluidic structure can include a porous hydrogel polymer material configured to partially enclose the one or more cells of the first type.

### Computer Systems

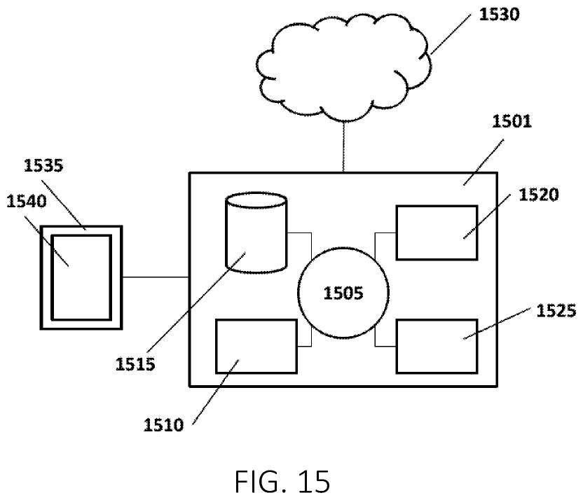

The present disclosure provides computer systems that are programmed to implement methods of the disclosure. FIG. 15 shows a computer system 1501 that may be programmed or otherwise configured to perform methods described herein. The computer system 1501 can regulate various aspects of the present disclosure, such as, for example, identifying a biological component, detecting a barcode, generating a spatial modulating element (e.g., a mask), providing energy from an energy source, or detecting or measuring a local parameter using a sensor. The detector may be a camera (e.g., a fluorescent camera), such as a charged coupled device (CCD) camera capable of collecting optical signals and position information from a plurality of sources distributed over a planar region. The computer system 1501 can be an electronic device of a user or a computer system that may be remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that may be in communication with the Internet. The network 1530 in some cases may be a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit may be an application specific integrated circuit (ASIC).

The storage unit 1515 can store files, such as drivers, libraries, and saved programs. The storage unit 1515 can store user data, e.g., user preferences and user programs. The computer system 1501 in some cases can include one or more additional data storage units that are external to the computer system 1501, such as located on a remote server that may be in communication with the computer system 1501 through an intranet or the Internet.

The computer system 1501 can communicate with one or more remote computer systems through the network 1530. For instance, the computer system 1501 can communicate with a remote computer system of a user (e.g., a laptop, a personal computer, a tablet, or a mobile phone). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that may be carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, an image of a biological component, a barcode, a signal or measurement of a local parameter. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, identify a biological component, detect a barcode, generate a spatial modulating element (e.g., a mask), provide energy from an energy source, detect or measure a local parameter using a sensor, etc.

While the systems and methods described herein have been described with reference to several particular example embodiments, those skilled in the art will recognize that many changes may be made thereto without departing from the spirit and scope of the present disclosure. The systems and methods described herein is applicable to a variety of sensor implementations and other subject matter, in addition to those discussed above.

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6th edition (Saunders, 2007).

“Chimeric antigen receptor T cell” (CAR-T cell) means a T cell engineered to express an antigen-specific receptor formed by fusing antigen-binding components with transmembrane and signaling components, which is capable of eliciting a cytotoxic T cell response whenever the antigen-specific component binds to its target. References teaching the manufacture and application of CAR-T cells include the following references that are incorporated herein by reference: U.S. Pat. No. 8,822,647; and U.S. patent publications 2014/0134142; 2014/0314795; 2012/0148552; 2013/0288368; and the like.

“Cleavable linkage” or “cleavable nucleotide” means any of wide variety of cleavable linkages, or more particularly, cleavable nucleotides, may be used with embodiments described herein. As used herein, the term “cleavable site” refers to a nucleotide or backbone linkage of a single stranded nucleic acid sequence that can be excised or cleaved under predetermined conditions, thereby separating the single stranded nucleic acid sequence into two parts. In some embodiments, a step of cleaving a cleavable nucleotide or a cleavable linkage leaves a free 3′-hydroxyl on a cleaved strand, thereby, for example permitting the cleaved strand to be extended by a polymerase. Cleaving steps may be carried out chemically, thermally, enzymatically or by light-based cleavage. Sometimes the term “releasing” may be used in reference to cleaving an oligonucleotide label, for example, by a releasing reagent or agent, which may be one or more of those listed above. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage by glycosylases and/or endonucleases may require a double stranded DNA substrate. Methods synthesizing and cleaving nucleic acids containing chemically cleavable, thermally cleavable, and photo-labile groups are described for example, in U.S. Pat. No. 5,700,642, which is incorporated herein by reference. Further cleavable linkages are disclosed in the following references: Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728, Urdea et al, U.S. Pat. No. 5,367,066, which are incorporated herein by reference. Synthesis and cleavage conditions of chemically cleavable oligonucleotides are described in U.S. Pat. Nos. 5,700,642 and 5,830,655. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide. In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide, which may (if desired) be removed by a phosphatase. In some embodiments, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al., J. Org. Chem. 61:525-529 (1996), Kahl et al., J. Org. Chem. 64:507-510 (1999), Kahl et al., J. Org. Chem. 63:4870-4871 (1998), Greenberg et al., J. Org. Chem. 59:746-753 (1994), Holmes et al., J. Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially. In some embodiments, ribonucleotides may be employed as cleavable nucleotides, wherein a cleavage step may be implemented using a ribonuclease, such as RNase H. In other embodiments, cleavage steps may be carried out by treatment with a nickase.

“Hydrogel” means a gel comprising a crosslinked hydrophilic polymer network with the ability to absorb and retain large amounts of water (for example, 60 to 90 percent water, or 70 to 80 percent) without dissolution due to the establishment of physical or chemical bonds between the polymeric chains, which may be covalent, ionic or hydrogen bonds. Hydrogels exhibit high permeability to the oxygen and nutrients, making them attractive materials for cell encapsulation and culturing applications. Hydrogels may comprise natural or synthetic polymers and may be reversible (i.e. degradable or depolymerizable) or irreversible. Example synthetic hydrogel polymers include polyethylene glycol (PEG), poly(2-hydroxyethyl methacrylate) and poly(vinyl alcohol). Example natural hydrogel polymers include alginate, hyaluronic acid and collagen. The following reference describe hydrogels and their biomedical uses: Drury et al, Biomaterials, 24:4337-4351 (2003); Garagorri et al, Acta Biomatter, 4 (5): 1139-1147 (2008); Caliari et al, Nature Methods, 13 (5): 405-414 (2016); Bowman et al, U.S. Pat. No. 9,631,092; Koh et al, Langmuir, 18 (7): 2459-2462 (2002).

Whenever the term “at least” precedes the first numerical value in a series of two or more numerical values, the term “at least” applies to each of the numerical values in that series of numerical values. For example, at least 1, 2, or 3 is equivalent to at least 1, at least 2, or at least 3.

Whenever the term “less than” precedes the first numerical value in a series of two or more numerical values, the term “less than” applies to each of the numerical values in that series of numerical values. For example, less than 3, 2, or 1 is equivalent to less than 3, less than 2, or less than 1.

The terms “coupled to,” “connected to,” and “in communication with,” as used herein, generally refer to any form of interaction between two or more entities, including mechanical, electrical, magnetic, electromagnetic, fluid, biological, and thermal interaction. Two components may be coupled to each other even though they are not in direct contact with each other.

The terms “polypeptide” and “peptide,” as used interchangeably herein, generally refer to a polymer of amino acids in which an amino acid may be linked to another amino acid by a peptide bond. In some examples, a polypeptide is a protein. The amino acid may be a naturally occurring amino acid or a non-naturally occurring amino acid (e.g., an amino acid analogue). The polypeptide can be linear or branched. The polypeptide can include modified amino acids. The polypeptide may be interrupted by non-amino acids. A polypeptide can occur as a single chain or an associated chain. The polypeptide may include a plurality of amino acids. The polypeptide may have a secondary and tertiary structure (e.g., the polypeptide may be a protein). In some examples, the polypeptide can comprise at least 2, 3, 4, 5, 6, 7, 8, 9, 10, 20, 30, 40, 50, 100, 1,000, 10,000, or more amino acids. The polypeptide may be a fragment of a larger polymer. In some examples, the polypeptide can be a fragment of a larger polypeptide, such as a fragment of a protein.

The term “amino acid,” as used herein, generally refers to a naturally occurring or non-naturally occurring amino acid (e.g., an amino acid analogue). The non-naturally occurring amino acid may be an engineered or synthesized amino acid.

The term “sample,” as used herein, generally refers to a chemical or biological sample containing a biological component. The biological component may comprise a cell, a nucleic acid, a microbiome, a protein, a combination of cells, a metabolite, a combination thereof, or any other suitable component of a biological sample. For example, a sample can be a biological sample including one or more cells. For another example, a sample can be a biological sample including one or more polypeptides. The biological sample can be obtained (e.g., extracted or isolated) from or include blood (e.g., whole blood), plasma, serum, urine, saliva, mucosal excretions, sputum, stool, and tears. The biological sample can be a fluid or tissue sample (e.g., skin sample). In some instances, the sample may be derived from a homogenized tissue sample (e.g., brain homogenate, liver homogenate, or kidney homogenate). In certain embodiments, the sample may include a specific type of cell (e.g., a neuronal cell, muscle cell, liver cell, or kidney cell,). The sample may comprise or be acquired from a diseased cell or tissue (e.g., a tumor cell or a necrotic cell), In some embodiments, the sample may include or may be from a disease-associated inclusion (e.g., a plaque, a biofilm, a tumor, or a non-cancerous growth). In certain embodiments, the sample may include or may be obtained from a cell-free bodily fluid, such as whole blood, saliva, or urine. In various embodiments, the sample can include circulating tumor cells. In some cases, the sample may include or may be an environmental sample (e.g., soil, waste, or ambient air), industrial sample (e.g., samples from any industrial processes), or a food sample (e.g., dairy product, vegetable product, or meat product). The sample may be processed prior to loading into a microfluidic device. For example, the sample may be processed to purify a certain cell type or polypeptide and/or to include reagents.

In some embodiments, as used herein, the term “local parameter” means a value of a parameter (such as, pH) in or immediately adjacent to a chamber formed by polymer matrix walls.

“On demand” means an operation may be directed to individual, discrete, selected locations (e.g. a spatial location of polymer precursor solution; or a selected polymer matrix chamber). Such selection may be based on manual observation of optical signals or data collected by a detector, or such selection may be based on a computer algorithm operating on optical signals or data collected by a detector. Manual observation of optical signals or data collected by a detector can include either real-time detection or detection at a time period prior to modulating a unit of energy to polymerize polymer precursors or degrading a chamber. For example, a subset of chambers (all formed with photo-degradable polymer matrix walls) may be pre-selected for releasing and removing their contents based on position information and the values of optical signals from an analytical assay carried out in the chambers. The pre-selected chambers may be photo-degraded by selectively projecting a light beam of appropriate wavelength characteristics (for example, with the spatial energy modulating element) to degrade the polymer matrix walls of the pre-selected chambers. In another embodiment, the pre-selected chambers may be photo-degraded by selectively projecting a light beam of appropriate wavelength characteristics (for example, with the spatial energy modulating element) in the presence of a photoinitiator to degrade the polymer matrix walls of the pre-selected chambers. Examples of a photoinitiator includes one of lithium phenyl-2,4,6-trimethylbenzoylphosphinate (LAP), Irgacure 2959, diphenyl(2,4,6-trimethylbenzoyl)phosphine oxide (TPO) nanoparticles, 2,2′-azobis [2-methyl-N-(2-hydroxyethyl) promionamide] (VA-086), BAPO-Oli, BAPO-Ona, Eosin-Y, Riboflavin, and combination thereof. In another example, a plurality of chambers may be observed in real-time (e.g. via fluorescent microscopy) for detection of an analyte of interest and one or more chambers of the plurality of chambers is selected, in real-time, upon detection of the analyte of interest, for degradation.

“Physical photomask” generally refers to a physical structure having a plurality of apertures or holes through which light may be projected. Physical photomasks can be used to create hydrogel matrices as described herein by causing the polymer precursor solution to polymerize and forming three-dimensional structures that correspond to the pattern on the photomask. A physical photomask can be patterned with a specific layout or geometric pattern. A physical photomask may be adhered to the upper surface of a flow cell.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90 ° C., primers annealed at a temperature in the range 50-75 ° C., and primers extended at a temperature in the range 72-78 °C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred uL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” or “quantitative PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30:1292-1305 (2002), which is also incorporated herein by reference.

“Polymer matrix” generally refers to a phase material (e.g. continuous phase material) that comprises at least one polymer. In some embodiments, the polymer matrix refers to the at least one polymer as well as the space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid, or gaseous species. For example, the term “polymer matrix” may encompass desiccated hydrogels, hydrated hydrogels, and hydrogels containing glass fibers. A polymer matrix may comprise one or more polymer precursors, which generally refers to one or more molecules that upon activation can trigger or initiate a polymeric reaction. A polymer precursor can be activated by electrochemical energy, photochemical energy, a photon, magnetic energy, or any other suitable energy. As used herein, the term “polymer precursor” includes monomers (that are polymerized to produce a polymer matrix), porogens, and crosslinking compounds, which may include photo-initiators, other compounds necessary or useful for generating polymer matrices, especially polymer matrices that are hydrogels. A polymer matrix may be semi permeable so that cells and beads (e.g., ranging from 3 to 50 microns) are too big to pass through, but smaller reagents can pass through such as antibodies, buffering salts, cellular media, lysing agents.

“Transfection” and/or “transformation” and/or “transduction” are used synonymously herein mean the transfer of exogenous genetic material to a target mammalian cell. Such transfer may be result in temporary or transient expression of a transgene or temporary or transient transcription of an RNA, for example, because of exhaustion of genetic material, loss or degradation of genetic material, lack of replication of genetic material, or the like. In some embodiments, “transfection” means “stable transfection” as the latter term is commonly used, e.g. Kim et al, Anal. Bioanal. Chem., 379:3173-3178 (2010). Exogenous genetic material may include plasmids, viral vectors, transgenes, transposons, or the like. “Stable” as used herein means that the exogenous genetic material persists through multiple cell divisions or for the life of the cellular host. The exogenous genetic material may be integrated into the genome of a target mammalian cell or it may comprise episomal DNA, such as a plasmid.

The following paragraphs overview certain aspects of the present disclosure:

A1. A method of analyzing cells, the method comprising:
loading a channel with (i) one or more cells of a first type, (ii) one or more cells of a second type, and (iii) one or more polymer precursors; polymerizing the one or more polymer precursors to form a chamber that encloses the one or more cells of the first type and the one or more cells of the second type, wherein the chamber is within the channel, wherein the one or more cells of the first type and the one or more cells of the second type both cannot flow through a wall of the chamber from an interior of the chamber to an exterior of the chamber;
after the polymerizing the one or more polymer precursors to form the chamber, loading the channel with a medium containing a component,
after loading the channel with the medium, measuring a cd71 of the chamber of the one or more cells of a first type and/or one or more cells of a second type.

A2. The method of aspect A1, wherein the one or more cells of the first type comprise antigen receiving cells.

A3. The method of aspect A1, wherein the one or more cells of the first type comprise T-cells.

A4. The method of aspect A1, wherein the one or more cells of the first type comprise OT1 cells.

A5. The method of any one of aspects A1-A4, wherein the one or more cells of the second type comprise antigen receiving cells.

A6. The method of any one of aspects A1-A5, wherein the one or more cells of the second type comprise dendritic cells.

A7. The method of any one of aspects A1-A6, wherein the component is selected from the group consisting of an activator, inhibitor, activated cell, non-activated cell, growth factor, and combinations thereof.

A8. The method of any one of aspects A1-A7, wherein the component comprises activated OT1 cells, wherein the activated OT1 cells cannot pass through the wall of the chamber from the exterior of the chamber to the interior of the chamber.

A9. The method of aspect A8, wherein the activated OT1 cells secrete a signaling chemical that interacts with the one or more cells of the first type or the second type within the chamber to have an increased cell life compared to a situation where there is an absence of the activated OT1 cells proximate to the exterior of the chamber.

A10. The method of aspect A9, wherein the signaling chemical comprises a growth factor.

A11. The method of any one of aspects A1-A9, wherein the characteristic is selected from the group consisting of (i) a change in total number of cells of the first type and/or the second type during a time duration, (ii) an activation of the one or more cells of the first type and/or the second type during the time duration, (iii) a change in the total number of live cells of the first type and/or the second type during the time duration, (iv) a change in the total number of dead cells of the first type and/or the second type during the time duration, and (v) combinations thereof.

A12. The method of any one of aspects A1-A11, wherein the one or more cells of the first type physically contact the one or more cells of the second type within the chamber to form an activated state of either the one or more cells of the first type and/or the one or more cells of the second type.

A13. The method of any one of aspects A6-A7, wherein the dendritic cells comprise GFP and the OT1 cells are in an activated state, wherein the OT1 cell in the chamber produce CD71 antigen.

A14. The method of any one of aspects A1-A13, wherein the loading the channel with the medium comprises flowing non-activated OT1 cells and phorbol myristate acetate (PMA).

A15. The method of any one of aspect A1-A14 further comprises:
after the polymerizing the one or more polymer precursors, measuring a first total number of cells of the first type or second type within the chamber; and
after a predetermined time period, measuring a second total number of cells of the first type or second type within the chamber.

A16. A method of culturing T-cells, the method comprising:
loading a channel with one or more cells and one or more polymer precursors;
polymerizing the one or more polymer precursors to form a chamber that encloses the one or more cells, wherein the chamber is within the channel, wherein the one or more cells cannot flow through a wall of the chamber from an interior of the chamber to an exterior of the chamber;
after the polymerizing the one or more polymer precursors to form the chamber, loading the channel with a medium containing a component,
after loading the channel with the medium, measuring a characteristic of the chamber of the one or more cells enclosed in the chamber.

A17. The method of aspect A16, wherein the characteristic corresponds to a total number of the cells increasing within the chamber through cell division after the loading the channel with the medium.

A18. The method of aspect A16, wherein the one or more cells comprise T-cells.

A19. The method of aspect A16, wherein the one or more cells comprise OT1 cells.

A20. The method of any one of aspects A16-19, wherein the component is selected from the group consisting of an activator, inhibitor, activated cell, non-activated cell, growth factor, and combinations thereof.

A21. The method of any one of aspects A16-A20, wherein the component comprises activated OT1 cells, wherein the activated OT1 cells cannot pass through the wall of the chamber from the exterior of the chamber to the interior of the chamber.

A22. The method of aspect A21, wherein the activated OT1 cells secrete a signaling chemical that interacts with the one or more cells within the chamber to have an increased cell life compared to a situation where there is an absence of the activated OT1 cells proximate to the exterior of the chamber.

A23. The method of aspect A22, wherein the signaling chemical comprises a growth factor.

A24. The method of any one of aspects A16-A23, wherein the characteristic is selected from the group consisting of (i) a change in total number of cells during a time duration, (ii) an activation of the one or more cells during the time duration, (iii) a change in the total number of live cells during the time duration, (iv) a change in the total number of dead cells during the time duration, and (v) combinations thereof.

A25. The method of any one of aspects A16-A24, wherein the OT1 cells in the chamber produce CD71 antigen.

A26. The method of any one of aspects A16-A25, wherein the loading the channel with the medium comprises flowing non-activated OT1 cells and phorbol myristate acetate (PMA).

A27. The method of any one of aspect A16-A26 further comprises:
after the polymerizing the one or more polymer precursors, measuring a first total number of cells within the chamber; and
after a predetermined time period, measuring a second total number of cells within the chamber.

A28. A method of analyzing cells, the method comprising: loading a channel with (i) one or more cells of a first type, (ii) one or more cells of a second type, and (iii) one or more polymer precursors;
polymerizing the one or more polymer precursors to form a first chamber that encloses the one or more cells of the first type, wherein the first chamber is within the channel,
polymerizing the one or more polymer precursors to form a second chamber that encloses the one or more cells of the second type, wherein the second chamber is within the channel,
wherein the one or more cells of the first type cannot flow through a wall of the first chamber from an interior of the first chamber to an exterior of the first chamber;
wherein the one or more cells of the second type cannot flow through a wall of the second chamber from an interior of the second chamber to an exterior of the second chamber;
after the polymerizing the one or more polymer precursors to form the first chamber and the second chamber, loading the channel with a medium containing a component,
after loading the channel with the medium, measuring a characteristic of the one or more cells of the first type in the first chamber and/or the one or more cells of a second type in the second chamber.

A29. The method of aspect A28, wherein the first chamber and the second chamber are disposed in a spaced apart arrangement having a separation distance ranging from 5 to 1000 microns.

A30. The method of any one of aspects A28-A29, wherein the first chamber and the second chamber each have an inner diameter ranging from 10 to 500 microns.

A31. The method of any one of aspects A28-A30, wherein the second chamber has an inner diameter that is 2 to 10 times greater than an inner diameter of the first chamber.

A32. A method of analyzing cells, the method comprising:
flowing one or more cells of a first type into a channel, wherein the channel comprises a fluidic structure configured to trap the flowing one or more cells of the first type at the fluidic structure;
flowing one or more cells of a second type into the channel along with a medium; and the one or more cells of the second type is not trapped by the fluidic structure.
measuring a characteristic of the chamber of the one or more cells of a first type and/or one or more cells of a second type.

A33. The method of aspect A32, wherein the first fluidic structure is configured to at least partially enclose the one or more cells of the first type.

A34. The method of any one of aspects A32-A33, wherein the first fluidic structure comprises a C-shaped structure to trap the one or more cells of the first type.

A35. The method of any one of aspects A32-A34, wherein the first fluidic structure comprises an array of pillars configured to trap the one or more cells of the first type.

A36. The method of any one of aspects A32-A35, wherein the channel comprises a first surface and second surface, wherein in an axis perpendicular to the first surface and/or the second surface, the fluidic structure does not touch both the first surface and second surface.

A37. The method of any one of aspects A32-A36, wherein the fluidic structure comprises a non-porous polymer material.

A38. The method of any one of aspects A32-A36, wherein the fluidic structure comprises a porous hydrogel polymer material configured to partially enclose the one or more cells of the first type.

A39. A method for processing a biological sample, comprising: (a) introducing one or more non-activated cells into a fluidic device; (b) introducing one or more polymer precursors into the fluidic device; (c) determining a location of a non-activated cell of the one or more cells within the fluidic device; (d) selectively polymerizing the polymer precursors to generate a polymer matrix from the polymer precursors within the fluidic device, wherein the polymer matrix at least partially encapsulates the cell; and (e) introducing one or more activated cells into the fluidic device, wherein the one or more activated cells remain external to the polymer matrix.

A40. The method of aspect A39, wherein the polymer matrix forms a chamber, and wherein an interior of a portion of the chamber comprises the non-activated cell.

A41. The method of aspect A40, wherein the activated cell is located exterior to the chamber.

A42. The method of aspect A39, wherein the polymer matrix forms a hydrogel.

A43. The method of aspect A39, wherein the fluidic device comprises a surface having a capture probe immobilized thereto, and wherein the capture probe couples to a biological component of the non-activated cell to immobilize the biological component to the surface.

A44. The method of aspect A43, wherein the capture probe comprises one or more functional groups capable of interacting with the biological component.

A45. The method of aspect A44, wherein the one or more functional groups comprise a complementary DNA sequence to target DNA or RNA of the biological component.

A46. The method of aspect A39, wherein the determining of the location of the cell within the fluidic device in (c) comprises using a detector to image the fluidic device.

A47. The method of aspect A46, wherein the detector is coupled to an energy source configured to emit energy.

A48. The method of aspect A47, wherein the energy source is a light generating device, and wherein the energy comprises light.

A49. The method of aspect A47, wherein the energy source is in optical communication with the fluidic device.

A50. The method of aspect A49, further comprising generating a virtual mask based on the location of the non-activated cell within the fluidic device determined in (c).

A51. The method of aspect A50, further comprising projecting the virtual mask using the light emitted from the light generating device.

A52. The method of aspect A51, wherein the light projected from the virtual mask is used to selectively polymerize the polymer precursors to generate the polymer matrix in (d).

A53. The method of any one of aspects A50-A52, wherein the virtual mask is generated from a spatial light modulator (SLM).

A54. The method of aspect A53, wherein the SLM is a digital micromirror device (DMD).

A55. The method of aspect A39, further comprising releasing an analyte from the cell.

A56. The method of aspect A55, wherein the analyte comprises messenger ribonucleic acids (RNAs), and wherein the method further comprises capturing the messenger RNAs with one or more capture oligonucleotides located on a portion of the fluidic device encompassed by the polymer matrix.

A57. The method of aspect A56, further comprising degrading the polymer matrix and loading the fluidic device with reverse transcriptase reagents to copy the captured messenger RNAs to produce complementary deoxyribonucleic acids (DNAs).

A58. The method of aspect A57, further comprising sequencing the complementary DNAs.

A59. The method of aspect A56, wherein the one or more capture oligonucleotides each comprise a barcode which indicates a position within the fluidic device.

A60. The method of any one of aspects A39-A59, wherein the polymer matrix comprises polymer matrix walls having an annular-like cross-section.

A61. The method of any one of aspects A39-A60, wherein the polymer matrix comprises further comprising performing one or more functional assays on the biological component, and wherein the one or more functional assays assess cell viability, cell morphology, cell secretions, cell responses, intercellular interactions, or any combination thereof.

A62. The method of aspect A61, wherein the one or more functional assays are performed using bright-field phase contrast or fluorescent imaging of the biological component.

A63. The method of any one of aspects A39-A62, further comprising, prior to (d), selecting the non-activated cell based on one or more optical signals therefrom.

A64. The method of any one of aspects A39-A63, wherein the fluidic device comprises a channel with an inlet and an outlet, wherein the channel comprises a first surface and a second surface disposed opposite one another across the channel, and wherein the polymer matrix forms a wall extending between the first surface and the second surface, thereby forming a chamber, and wherein the chamber encapsulates the cell.

A65. The method of any one of aspects A39-A64, wherein the polymer matrix comprises a hydrogel, and wherein the hydrogel comprises acrylamide and poly(ethylene glycol).

A66. The method of any one of aspects A39-A65, wherein the polymer matrix comprises a degradable polymer matrix, and wherein the method further comprises degrading the polymer matrix with a degradation agent selected from the group consisting of dithiothreitol (DTT), tris(2-chloroethyl)phosphate (TCEP), tetrahydropyran (THP), and sodium periodate (NaIO₄).

A67. The method of any one of aspects A39-A66, wherein the polymer matrix comprises a photo-degradable polymer matrix, and wherein the method further comprises photo-degrading the polymer matrix by selectively projecting a light beam.

A68. The method of any one of aspects A39-A67, wherein the introducing in (a) and the introducing in (b) are performed simultaneously.

A69. The method of any one of aspects A39-A68, wherein the introducing in (e) occurs subsequent to the selective polymerization in (d).

A70. The method of any one of aspects A39-A69, wherein the one or more non-activated cells comprise T cells.

A71. The method of any one of aspects A39-A69, wherein the one or more non-activated cells comprise OT1 cells.

A72. The method of any one of aspects A39-A69, wherein the one or more non-activated cells comprise OT1 cells, mouse T cells, human T cells, dendritic cells, PBMCs, T cells, CD4+ T cells, CD8+ T cells, B cells, NK cells, monocytes, regulatory T cells, engineered immune cells such as CAR-T cells, neuron cells, stem cells, iPSC, endothelial cells, epithelial cells, adipocytes, primary cells, primary tumor cells, or cancer stem cells (CSCs), or any combination thereof.

A73. The method of any one of aspects A39-A72, wherein the one or more activated cells comprise activated OT1 cells, activated T cells, activated B cells, macrophages, dendritic cells, monocytes, astrocytes, microglia, stromal cells, feeder cells, cancer associated fibroblast (CAF), or tumor associated macrophages (TAM), or any combination thereof.

A74. The method of any one of aspects A39-A73, the one or more activated cells comprise activated OT1 cells, and wherein the activated OT1 cells secrete a signaling chemical that interacts with the non-activated cell within the chamber such that the non-activated cell has an increased cell life compared to a situation where there is an absence of the activated OT1 cells proximate to the exterior of the chamber.

A75. The method of aspect A74, wherein the signaling chemical comprises a growth factor.

What is claimed is:

1. A method, comprising:
measuring a cellular characteristic of a first cell in a fluidic device,
wherein the first cell is located in an interior of a compartment of the fluidic device and a second cell is located exterior to the compartment,
wherein the first cell cannot pass from the interior of the compartment to the exterior of the compartment,
wherein the second cell cannot pass from the exterior of the compartment to the interior of the compartment,
wherein a signaling chemical secreted by the second cell can pass from the exterior of the compartment to the interior of the compartment, and
wherein the signaling chemical interacts with the first cell, thereby modifying the cellular characteristic of the first cell.

2. The method of claim 1, wherein the characteristic comprises a cell life of the first cell.

3. The method of claim 1, wherein the characteristic comprises a division of the first cell.

4. The method of claim 1, wherein the interaction of the first cell with the signaling chemical causes an increased cell life of the first cell compared to a situation where there is an absence of the signaling chemical in the compartment.

5. The method of claim 1, wherein the interaction of the first cell with the signaling chemical causes an increased cell division of the first cell compared to a situation where there is an absence of the signaling chemical in the compartment.

6. The method of claim 1, wherein the measuring the cellular characteristic in (b) comprises measuring a change in a total number of cells in the compartment during a time duration.

7. The method of claim 1, wherein the measuring the cellular characteristic in (b) comprises measuring a change in a total number of alive cells in the compartment during a time duration.

8. The method of claim 1, wherein the measuring the cellular characteristic in (b) comprises measuring a change in a total number of dead cells in the compartment during a time duration.

9. The method of claim 1, wherein the cellular characteristic comprises activation.

10. The method of claim 1, wherein the measuring the cellular characteristic comprises measuring an antigen produced by the first cell.

11. The method of claim 10, wherein the antigen is CD71.

12. The method of claim 10, wherein the antigen comprises CD69, CD71, CD25, CD107a, 4-1BB, PD-1, CD80, CD86, CD83, CD34, CD133, SSEA4, TRA-1-60, EpCAM, or CA9.

13. The method of claim 1, wherein the first cell comprises an antigen receiving cell.

14. The method of claim 1, wherein the first cell comprises a mouse T cell, a human T cell, a dendritic cell, a PBMC, a T cell, a CD4+ T cell, a CD8+ T cell, a B cell, a NK cell, a monocyte, a regulatory T cell, an engineered immune cell, a CAR-T cell, a neuron cell, a stem cell, an induced pluripotent stem cell (iPSC), an endothelial cell, an epithelial cell, an adipocyte, a primary cell, a primary tumor cell, or a cancer stem cell (CSC).

15. The method of claim 1, wherein the first cell comprises a T-cell.

16. The method of claim 15, wherein the T-cell comprises an OT1 cell.

17. The method of claim 1, wherein the second cell comprises an antigen receiving cell.

18. The method of claim 1, wherein the second cell comprises an activated T cell, an activated B cell, a macrophage, a dendritic cell, a monocyte, an astrocyte, a microglia, a stromal cell, a feeder cell, a cancer associated fibroblast (CAF), or a tumor associated macrophage (TAM).

19. The method of claim 1, wherein the first cell and the second cell are a same type of cell.

20. The method of claim 1, wherein the first cell and the second cell are a different type of cell.

21. The method of claim 1, wherein the second cell comprises an activated T-cell.

22. The method of claim 21, wherein the activated T-cell comprises an activated OT1 cell.

23. The method of claim 1, wherein the signaling chemical comprises a growth factor.

24. The method of claim 1, wherein the compartment further comprises a third cell.

25. The method of claim 24, wherein the first cell is non-activated prior to interaction with the signaling chemical, and wherein the third cell comprises a dendritic cell.

26. The method of claim 25, wherein the dendritic cell stimulates the first cell to produce an antigen.

27. The method of claim 21, wherein the method further comprises loading the fluidic device with a medium comprising a non-activated T cell and phorbol myristate acetate (PMA),
wherein subsequent to the loading of the fluidic device with the medium comprising the non-activated T cell and the PMA, the non-activated T cell is activated, thereby obtaining the activated T cell.

28. The method of claim 1, wherein the second cell is in an interstitial space of the fluidic device.

29. The method of claim 1, wherein the second cell is located in an interior of an additional compartment.
